(19) United States
(12) Patent Application Publication
(10) Pub. No.: US 2024/0269287 A1
(43) Pub. Date: Aug. 15, 2024

(54) MICROBUBBLE-ASSISTED ULTRASOUND-GUIDED THERAPY

(71) Applicant: THE BOARD OF REGENTS OF THE UNIVERSITY OF TEXAS SYSTEM, AUSTIN, TX (US)

(72) Inventors: SINA KHORSANDI, DALLAS, TX (US); JACQUES LUX, PLANO, TX (US); WEN JIANG, HOUSTON, TX (US)

(73) Assignee: THE BOARD OF REGENTS OF THE UNIVERSITY OF TEXAS SYSTEM, AUSTIN, TX (US)

(21) Appl. No.: 18/554,944

(22) PCT Filed: Apr. 12, 2022
(86) PCT No.: PCT/US2022/024485

§ 371 Date: Oct. 11, 2023

Related U.S. Application Data
(60) Provisional application No. 63/316,360, filed on Mar. 3, 2022.
(60) Provisional application No. 63/173,956, filed on Apr. 12, 2021.

(51) Int. Cl. A61K 41/00; A61K 47/54; A61K 47/61; A61K 47/68; A61K 47/69; A61N 7/00

(52) U.S. Cl. CPC A61K 41/0028; A61K 47/543; A61K 47/61; A61K 47/6849; A61K 47/6925; A61N 7/00; A61N 2007/0039

(57) ABSTRACT

The innate immune sensing STING pathway has emerged as a potential therapeutic target to boost antitumor immune responses. STING resides in the cytoplasm, and its agonists, such as cGAMP, are dinucleotides that are difficult to deliver intracellularly. Disclosed herein is a microbubble-based platform (Microbubble (MB)-assisted UltraSound (US)-guided Immunotherapy of Cancer (MUSIC)) that can be used for targeted activation of STING, such as for treatment of primary and metastatic tumors.

### CROSS-REFERENCE TO RELATED APPLICATIONS

This application claims the benefit of priority to U.S. Provisional Application No. 63/173,956, filed on Apr. 12, 2021 and U.S. Provisional Application No. 63/316,360, filed on Mar. 3, 2022, the entire contents of which are hereby incorporated herein by reference.

### BACKGROUND

Cyclic dinucleotides (CDNs) have been found to have interesting immune-stimulatory properties through their activation of Stimulator of Interferon Genes (STING). CDNs produced by bacteria elicit an innate immune response that is critical for effective host defense against infection. However, as small molecules, CDNs are rapidly flushed from the injection site, leading to systemic inflammatory side effects. Furthermore, poor CDN internalization and localization in the cytosolic compartments of cells presents a significant barrier to the potential of CDN-based therapeutics. Therefore, there remains a need for strategies effecting targeted and effective intracellular delivery of CDNs.

### SUMMARY

According to one aspect of the disclosure, provided herein is a method of targeted in vitro or in vivo drug delivery using sonoporation. The method entails administering to one or more target cells a composition comprising microbubbles loaded with a payload and then administering an ultrasound stimulus to the one or more target cells. The ultrasound stimulus is effective to sonoporate the one or more target cells.

The payload may be an agonist for activating the Stimulator of Interferon Genes (STING) signaling pathway within the one or more target cells, such as a cyclic dinucleotide. The payload may be a cyclic dinucleotide for inducing or enhancing Type 1 Interferon production within one or more cells. The payload may be mRNA. The payload may be DNA, such as plasmid DNA (pDNA).

The method may be an in vivo method which entails administering the microbubble composition and the ultrasound stimulus to a subject. The one or more target cells may be cancer cells. The one or more target cells may be immune cells. The immune cells may be professional antigen-presenting cells (APCs), such as macrophages and/or dendritic cells.

The microbubbles may include targeting molecules on the external surfaces of the microbubbles that are effective to bind the one or more target cells. The targeting molecules may be antibodies. The targeting molecules may bind CD11b.

The ultrasound stimulus may be administered at about 1-2 W/cm², optionally with 50% duty cycle. The ultrasound stimulus may be administered for between about at least about 30-60 seconds. The one or more target cells may be exposed to the microbubbles for at least about 10 minutes prior to administering the ultrasound stimulus. The method may further entail using ultrasound to visualize the microbubbles prior to applying the ultrasound stimulus effective to sonoporate the cell membrane. The intensity of the ultrasound used to visualize the microbubbles can be less than the intensity of the ultrasound stimulus.

The microbubbles may be decorated with spermine and the payload may be non-covalently bound to the spermine. The microbubbles may be decorated with spermine-dextran conjugates and the payload non-covalently bound to the spermine within the spermine-dextran conjugates. The microbubbles may have gas cores having a perfluorocarbon. The perfluorocarbon may be decafluorobutane. The microbubbles may have shells having phospholipids, optionally wherein the phospholipids include one or both of 1,2-Distearoyl-sn-Glycero-3-Phosphocholine (DSPC) and 1,2-Distearoyl-sn-Glycero-3-Phosphoethanolamine (DSPE) lipids. The microbubbles may have surfactant shells having PEGylated molecules.

The average microbubble size of the microbubble composition may be between about 1 μm and about 10 μm. The average microbubble size of the microbubble composition may be between about 1 μm and about 5 μm. The average microbubble size of the microbubble composition may be about 3 μm.

The microbubbles within the microbubble composition may be primarily nanobubbles or entirely nanobubbles. The average microbubble size of the microbubble composition may be between about 100 nm and 700 nm. The average microbubble size of the microbubble composition may be between about 200 nm and 600 nm. The average microbubble size of the microbubble composition may be between about 300 nm and 500 nm.

The microbubble composition may include microbubbles with biodegradable linkers operably positioned between an exterior surface of a shell of the microbubble and the payload. The biodegradable linkers may be joining a spermine to a dextran or a spermine to another spermine.

The payload may be cyclic guanosine monophosphate-adenosine monophosphate (cGAMP).

The method may be an in vitro method. The microbubble composition may be incubated with the one or more target cells at a concentration of at least about 5, 10, 15, 20, 25, or 30 microbubbles/cell. The step of administering the microbubble composition to the one or more target cells may entail mixing the composition with the one or more target cells in solution. The one or more target cells may be adhered to a surface and the step of administering the microbubble composition to the one or more target cells may entail exposing the surface to the composition in a manner such that the one or more cells are positioned over the microbubbles.

According to another aspect of the disclosure, provided herein is a method of treating cancer in a subject in need thereof. The method entails performing any of the aforementioned targeted drug delivery methods, in which administering the microbubble composition to the one or more target cells entails administering the microbubble composition and the ultrasound stimulus to the subject in need of treatment, and in which the payload is a cyclic dinucleotide.

The subject may have been diagnosed with cancer. The subject may have a tumor. The subject may have one or more metastases. The microbubble composition may be administered intratumorally. The microbubble composition may be administered systemically. The microbubble composition may be administered intravenously. The microbubble composition may be a nanobubble composition.

Administering the microbubble composition to the subject may entail administering multiple doses of the microbubble composition to the subject and administering the ultrasound stimulus to the subject may entail administering ultrasound stimulus effective to sonoporate the one or more target cells after each dose. The multiple doses may be administered at least one day apart.

The administration may result in an increase in expression of IFN-α, IFN-β, and/or IFN-γ within the one or more target cells. The administration may result in an increase in serum levels of IFN-α, IFN-β, and/or IFN-γ. The administration may result in nuclear localization of nuclear translocation of phosphorylated TRF3 (pIRF3) and/or NF-κB p65 in the one or more target cells. The administration may result in increased recruitment of CD8+ and CD4+ T cells within the tumor. The administration may result in an increased number of effector memory T-cells and/or central memory T-cells that are specific to cancer cells within the subject. An increased number of effector memory T-cells and/or central memory T-cells may be found, specifically, within the tumor. The administration may result in a decrease in tumor size. The administration may result in the eradication of the tumor. The administration may prevent or reduce the likelihood of future metastases. The administration may prevent or reduce the likelihood of recurrence of the cancer in the subject. The method may further entail treating the subject with immune checkpoint therapy. The immune checkpoint therapy may entail administering to the subject inhibitors that target CTLA4, PD-1, PD-L1, and/or CD47.

According to another aspect of the disclosure, provided herein is a microbubble composition for therapeutic drug delivery. The microbubble composition includes a plurality of microbubbles and a plurality of cyclic dinucleotides. The microbubbles each have a gas core encapsulated by a surfactant shell. A plurality of cationic polymers is associated with the external surface of the surfactant shell of each microbubble. The cyclic dinucleotides are non-covalently bound to the cationic polymers on the external surface of the microbubbles.

The cationic polymers may be polyamines. The polyamines may be spermines. The spermines may be conjugated to dextrans. Multiple spermines may be conjugated to each dextran. The plurality of cyclic dinucleotides may include cyclic guanosine monophosphate-adenosine monophosphate (cGAMP).

According to another aspect of the disclosure, provided herein is a method of the aforementioned microbubble compositions. The method entails associating the cationic polymers with the microbubbles and loading the cyclic dinucleotides onto the microbubbles after the cationic polymers have been associated.

According to another aspect of the disclosure, provided herein is another method of making the aforementioned microbubble composition. The method entails binding the cyclic dinucleotides to the cationic polymers to form nanocomplexes and loading the nanocomplexes onto the microbubbles.

According to another aspect of the disclosure, provided herein is the microbubble composition formed by either of the aforementioned methods of making microbubble compositions.

According to another aspect of the disclosure, provided herein is the use of any one of the aforementioned microbubble compositions in any one of the aforementioned methods of drug delivery and/or treatment of cancer.

### BRIEF DESCRIPTION OF THE DRAWINGS

FIGS. 1A-1B depict size distributions of SpeDex-aCD11b MBs (cMBs) measured by Coulter Counter (FIG. 1A) and SpeDex-cGAMP nanocomplexes measured using nanoparticle tracking analysis (FIG. 1i).

FIG. 2 depicts mean fluorescence intensity quantification of the DY547-c-diGMP uptake in BMDMs.

FIG. 3 depicts quantification by flow cytometry of live and dead THP-1 and EO771 cells stained with Propidium Iodide (PI) after sonoporation with ncMBs at 1, 2, and 3 W/cm².

FIGS. 4A-4H depict qRT-PCR results showing fold changes in levels of: IFNα mRNA in THP-1 macrophages at various time points after treatment (FIG. 4A), IFNβ mRNA in THP-1 macrophages at various time points after treatment (FIG. 4B), IFNα mRNA in BMDMs at various time points after treatment (FIG. 4C), IFNβ mRNA in BMDMs at various time points after treatment (FIG. 4D), IFNα mRNA in wild type and STING^{-/-} BMDMs at 6 h post-treatment (FIG. 4E), IFNβ mRNA in wild type and STING^{-/-} BMDMs at 6 h post-treatment (FIG. 4F), IFNα mRNA in primary human peripheral blood monocyte derived macrophages at 6 h post-treatment (FIG. 4G), and IFNβ mRNA in primary human peripheral blood monocyte derived macrophages at 6 h post-treatment (FIG. 4H).

FIGS. 5A-5H depict ELISA results showing protein levels in cell supernatants for: IFNα from THP-1 macrophages at various time points after treatment (FIG. 5A), IFNβ from THP-1 macrophages at various time points after treatment (FIG. 5B), IFNα from BMDMs at various time points after treatment (FIG. 5C), IFNβ from BMDMs at various time points after treatment (FIG. 5D), IFNα from wild type and STING^{-/-} BMDMs at 6 h post-treatment (FIG. 5E), IFNβ from wild type and STING^{-/-} BMDMs at 6 h post-treatment (FIG. 5F), IFNα from primary human peripheral blood monocyte derived macrophages at 6 h post-treatment (FIG. 5G), and IFNβ from primary human peripheral blood monocyte derived macrophages at 6 h post-treatment (FIG. 5H).

FIGS. 6A-6D depict western blot results showing STING, phosphorylated STING (pSTING^{SER366} or pSTING^{SER366}), IRF3, phosphorylated IRF3 (pIRF3^{SER366} or pIRF3^{SER396}) and R-actin (control) levels in: THP-1 macrophages at various time points after treatment (FIG. 6A), BMDMs at various time points after treatment (FIG. 6B), wild type and STING^{-/-} BMDMs at 6 h post-treatment (FIG. 6C), and primary human peripheral blood monocyte derived macrophages at 6 h post-treatment (FIG. 6D).

FIGS. 7A-7C depict western blot results showing IKKα, IKKβ, phosphorylated IKKα/β (Ser176/180), IκBα, phosphorylated IκBα (Ser32), NF-κB p65, phosphorylated NF-κB p65 (Ser536), and β-actin (control) levels in: THP-1 macrophages at various time points after treatment (FIG. 7A), BMDMs at various time points after treatment (FIG. 7B), and wild type and STING^{-/-} BMDMs at 6 h post-treatment (FIG. 7C).

FIGS. 8A-8C illustrate the nuclear translocation of phosphorylated IRF3 (pIRF3) following various treatment. FIG. 8A depicts immunohistochemistry images of stained nuclei of wild type BMDMs, STING^{-/-} BMDMs, THP-1 macrophages, and peripheral blood monocyte derived macrophages at 6 h post-treatment with the additional staining of phospho-IRF3. FIG. 8B depicts the percentage of nuclear fluorescent positive BMDMs and FIG. 8C depicts the percentage of nuclear fluorescent positive THP-1 cells.

FIGS. 9A-9C illustrate the nuclear translocation of NF-κB p65 following treatment. FIG. 9A depicts immunohistochemistry images of stained nuclei of wild type BMDMs, STING^{-/-} BMDMs, and THP-1 macrophages at 6 h post-treatment with the additional staining of NF-κB p65. FIG. 9B depicts the percentage of nuclear fluorescent positive BMDMs and FIG. 9C depicts the percentage of nuclear fluorescent positive THP-1 cells.

FIG. 10 depicts the quantification by flow cytometry of the percentage of EO771 breast cancer cells that were phagocytized by BMDMs, which were treated 6 h before being co-cultured with the EO771 cells for 4 h.

FIGS. 11A-11B depict normalized levels of proliferation, as measured by flow cytometry, of CD8⁺ (FIG. 11A) and CD4⁺ T cells (FIG. 11B) from OT-I and OT-II transgenic mice, respectively, that recognize the cOVA-derived peptide antigens, after incubation for 72 h with STING^{-/-} and wild type BMDMs that had been treated and subsequently incubated with the OVA peptide antigens for 6 hours.

FIGS. 12A-12C depict CD8⁺ T cell proliferation and cytokine levels for treated TAMs. FIG. 12A depicts normalized levels of proliferation, as measured by flow cytometry, of CD8⁺ T cells from OT-I transgenic mice after incubation for 72 h with TAMs that had been treated and subsequently incubated with the OVA peptide antigens for 6 hours. FIGS. 12B-12C depict ELISA results showing protein levels in cell supernatants for IFNα (FIG. 12B) and IFNβ (FIG. 12C) from wild type and STING^{-/-} BMDMs at 6 h post-treatment.

FIGS. 13A-13D illustrate IRF3/NF-κB activation in BMDCs. FIG. 13A depicts immunohistochemistry images of stained nuclei of wild type BMDCs and STING^{-/-} BMDCs at 6 h post-treatment with the additional staining of phospho-IRF3. FIG. 13B depicts the percentage of nuclear fluorescent positive BMDCs for phospho-IRF3. FIG. 13C depicts immunohistochemistry images of stained nuclei of wild type BMDCs and STING^{-/-} BMDCs at 6 h post-treatment with the additional staining of NF-κB p65. FIG. 13D depicts the percentage of nuclear fluorescent positive BMDCs for NF-κB p65.

FIGS. 14A-14D illustrate increased type I IFN responses in BMDCs at 6 h post-treatment. FIGS. 14A-14B depict qRT-PCR results showing fold changes in levels of IFNα mRNA (FIG. 14A) and IFNβ mRNA (FIG. 14B) in wild type and STING^{-/-} BMDCs. FIGS. 14C-14D depict ELISA results showing protein levels in cell supernatants for IFNα (FIG. 14C) and IFNβ (FIG. 14D) from wild type and STING^{-/-} BMDCs.

FIGS. 15A-15B depict normalized levels of proliferation, as measured by flow cytometry, of CD8⁺ (FIG. 15A) and CD4⁺ T cells (FIG. 15B) from OT-I and OT-II transgenic mice, respectively, after incubation for 72 h with STING^{-/-} and wild type BMDCs that had been treated and subsequently incubated with the OVA peptide antigens for 6 hours.

FIGS. 16A-16B illustrate the specificity of in vivo delivery of DY547-c-diGMP via treatment with MUSIC to tumor-associated CD11b+ cells in an orthotopic syngeneic murine breast cancer model relative to treatment with non-targeting microbubbles, as evaluated by flow cytometry performed on single cell suspensions. FIG. 16A depicts the percentage of CD11b⁺ cells that are positive for DY547-c-diGMP. FIG. 16B depicts the percentage of CD11b⁻ cells that are positive for DY547-c-diGMP.

FIG. 17 shows contrast mode ultrasound (US) images of EO771 breast tumors (13 days) in C57BL/6J mice before injection of ncMBs (left, non-treatment), after injection of ncMBs (middle, pre-sonoporation), and after US sonoporation (right, post-sonoporation). Loss in signal represents bubbles being destroyed after exposure to US. Images are from the same mouse, representative of randomly treated wild-type (WT) mice.

FIGS. 18A-18B depict the percentage of tumor area (day 18) that is CD11b+(FIG. 18A) and relative mean phosphorylated STING (pSTING) fluorescence intensity (FIG. 18B) as measured from confocal microscopy images of tumor paraffin section slides immunostained for recruited CD11b+ cells and pSTING+ cells.

FIGS. 19A-19B depict the percentage of CD11b+ cells (FIG. 19A) and TAMs (FIG. 19B) that are positive for pSTING as measured by flow cytometry performed on single cell suspensions of tumor tissue (day 18) from treated mice.

FIGS. 20A-20D illustrate the increased recruitment of CD8+ and CD4+ T cells into tumors (day 18) after MUSIC treatment. FIGS. 20A-15B depict the percentage of CD8+ T cells (FIG. 20A) and CD4+ T cells (FIG. 20B) detected in single cell suspensions of treated tumor tissue as measured by flow cytometry. FIGS. 20C-20D depict the percentage of tumor area that is CD8+(FIG. 20C) and CD4+(FIG. 20D) as measured from confocal microscopy images of immunostained tumor paraffin section slides.

FIGS. 21A-21I depict in vivo results of tumor treatment. FIGS. 21A-21D depict results in wild type mice, including spider plots of tumor volume for individual mice within each treatment group (FIG. 21A), cumulative group results for tumor volume (FIG. 21B), survival curves (FIG. 21C), and representative photographs of mice at 24 days post tumor inoculation (FIG. 21D). FIGS. 21E-21I depict results in STING^{-/-} mice, including average tumor volume (FIGS. 21E, 21H), survival curves (FIGS. 21F, 21I), and spider plots of tumor volume for individual mice (FIG. 21G).

FIG. 22 depicts tumor volumes for rechallenged MUSIC-treated mice that demonstrated complete tumor remission and non-treated naive mice (control).

FIGS. 23A and 23B depict tumor volumes (FIG. 23A) and survival curves (FIG. 23B) for STING^{-/-} mice treated with MUSIC or PBS (control).

FIGS. 24A-24C depict the average percentages, as measured by flow cytometry, of CD8+ T cells isolated from treated tumor tissue samples (day 18) that were naive T cells (FIG. 24A; CD44^{low}CD62L^{high}), effector memory T cells (FIG. 24B; CD44^{high}CD62L^{low}), and central memory T cells (FIG. 24C; CD44^{high}CD62L^{high}).

FIG. 25 depicts quantified IFN-γ enzyme-linked immune absorbent spot (ELISpot) results for splenic T cells that were isolated from treated mice (day 18) and rechallenged by co-culturing with EO771 tumor cells at a ratio of 10:1 (T cells:EO771 cells) overnight.

FIGS. 26A-26C depict in vivo results of tumor treatment in wild type mice and mice in which CD8⁺ cells were depleted using an anti-CD8 antibody prior to treatment, including spider plots of tumor volume for individual mice within each treatment group (FIG. 26A), mean tumor volumes (FIG. 26B), and survival rates (FIG. 26C).

FIGS. 27A-27F depict ELISA results showing protein levels measured in treated mice (18 days) from tumor tissue (FIGS. 27A-27C) and serum (FIGS. 27D-27F) for: IFNα (FIGS. 27A, 27D), IFNβ (FIGS. 27B, 27E), and IFN-γ (FIGS. 27C, 27F).

FIGS. 28A-28B depict the percentage of treated tumor area (18 days) that is CD8⁺ (FIG. 28A) and PD-1⁺ (FIG. 20D) as measured from confocal microscopy images of immunostained tumor paraffin section slides from wild type and STING^{-/-} mice.

FIGS. 29A-29B depict the relative mean fluorescence intensity for IFN-γ (FIG. 29A) and PD-L1 (FIG. 29B) in randomly selected confocal microscopy images of immunostained tumor paraffin section slides from tread wild type and STING^{-/-} mice.

FIGS. 30A-30C depict luminescence levels for bioluminescent tumor cells in mice treated with PBS only, MUSIC, cGAMP with US, cGAMP only, and MBs with US, including lung luminescence over time (FIG. 30A), final primary tumor and lung luminescence levels along with primary tumor volume and number of lung metastatic nodules (FIG. 30B), and heat mapping images of luminescence in extracted primary tumors, kidneys, livers, spleens, hearts, and lungs from representative mice (FIG. 30C).

FIGS. 31A-31U depict the effects of combination therapy with MUSIC and systemic administration of an anti-PD-1 antibody (aPD-1) in spontaneously metastatic murine triple negative 4T1 breast tumor-bearing mice. FIGS. 31A-31B depict relative bioluminescence intensity in the lungs (FIG. 31A) and primary tumor (FIG. 31B) of treated mice over time from representative in vivo images. FIGS. 31C-31E depicts spider plots of tumor volume for individual mice within each treatment group (FIG. 31C), mean tumor volumes (FIG. 31D), and survival rates (FIG. 31E). FIGS. 31F-31G depict the number of pulmonary metastatic modules (FIG. 31F) and relative bioluminescence intensity in the lungs of (FIG. 31G) treated mice (28 days) determined via ex vivo macroscopic organ imaging and examination. FIGS. 31H-31L depict ELISA results showing protein levels measured in treated mice from tumor tissue (FIGS. 31H-31I) and serum at 21 days post-inoculation (FIGS. 31J-31L) for: IFNα (FIGS. 31H, 31J), IFNβ (FIGS. 31I, 31K), and IFN-γ (FIG. 31L). FIGS. 31M-31N depict the percentage of CD8+ T cells (FIG. 31M) and CD4+ T cells (FIG. 31N) detected in single cell suspensions of treated tumor tissue as measured by flow cytometry. FIGS. 31O-31P depict the percentage of tumor area that is CD8+(FIG. 31O) and CD4+(FIG. 31P) as measured from confocal microscopy images of immunostained tumor paraffin section slides. FIGS. 31Q-31R depict relative mean phosphorylated STING (pSTING) (FIG. 31Q) and IFN-γ (FIG. 31R) fluorescence intensity levels as measured from confocal microscopy images of immunostained tumor paraffin section slides. FIGS. 31S-31U depict the average percentages, as measured by flow cytometry, of CD8⁺ T cells isolated from treated tumor tissue samples that were naive T cells (FIG. 31S; CD44^{low}CD62L^{high}), effector memory T cells (FIG. 31T; CD44^{high}CD62L^{low}), and central memory T cells (FIG. 31U; CD44^{high}CD62L^{high}).

FIG. 32 depicts the size distribution of nanobubbles (NBs) measured using nanoparticle tracking analysis.

FIGS. 33A-33B illustrate the successful cytosolic delivery of Dy547-diGMP to THP-1 macrophages via sonoporation with ncNBs. FIG. 33A depicts fluorescent images of macrophages treated via incubation with Dy547-diGMP only (left) or sonoporation with Dy547-diGMP-loaded ncNBs (right). FIG. 33B depicts the detection of the secreted embryonic alkaline phosphatase (SEAP) reporter in the THP-1 macrophage supernatant following sonoporation.

FIGS. 34A-34C illustrate the use of a clinical US scanner to detect the delivery of intravenously administered ncNBs to tumor tissue and sonoporate APCs within the tumor tissue. FIGS. 34A-34C depict schematics of the treated mouse and US images of the tumor site before (FIG. 34A), during (FIG. 34B), and after (FIG. 34C) injection of the ncNBs.

FIG. 35 depicts an agarose gel showing the complete binding of 200 ng of mRNA to 9 million SpeDex MBs (Lane 1=200 ng mRNA, Lane 2=100 ng mRNA, Lane 3=50 ng mRNA, Lane 4=25 ng mRNA, Lane 5=200 ng mRNA complexed to SpeDex MBs).

### DETAILED DESCRIPTION

Disclosed herein are microbubble compositions as well as methods of making and using such compositions. According to certain aspects of the disclosure, microbubble compositions may be used for targeted drug delivery (in vitro or in vivo) of a payload to one or more target cells. The payload may be delivered to the cytosol of the one or more targeted cells by targeted sonoporation. The microbubbles may also be targeted to the one or more target cells by incorporation of targeting molecules, such as antibodies, that bind to the one or more target cells. For instance, the microbubbles may target professional antigen presenting cells (APCs) such as macrophages and dendritic cells (e.g., by the incorporation of an anti-CD11b antibody). The payload may comprise any suitable agent that can be loaded onto the surface of the microbubbles. For example, the payload may be mRNA or plasmid DNA (pDNA) for use in applications such as nucleotide-based vaccines or gene therapies.

According to certain aspects of the disclosure, the payload may be an agonist of the cGAS-STING cytosolic DNA sensing pathway. The spatial and temporal targeting of STING activation afforded via sonoporation of drug-carrying microbubbles can allow for enhanced potency of STING agonists with decreased systemic inflammatory side effects and toxicity. Sonoporation is also particularly advantageous for delivering negatively charged payloads in general, such as nucleotides, across the negatively charged plasma membrane.

According to some aspects of the disclosure, such agonists may be used for the treatment of cancer, particularly solid tumor cancers (e.g., breast cancer, brain cancer, melanoma) or other cancers having solid masses (e.g., lymphomas). STING agonists may be used to activate downstream proinflammatory pathways in APCs that efficiently prime antigen-specific T cells, thus bridging innate and adaptive immune responses. Doing so can result in systemic antitumor immunity and/or antitumor memory responses. According to some aspects, the microbubble composition may be used as a vaccine, such as therapeutic vaccine for the treatment of cancer, or a vaccine adjuvant.

### Immune System

The human immune system may generally be divided into two arms, referred to as “innate immunity” and “adaptive immunity.” The innate arm of the immune system is predominantly responsible for an initial inflammatory response via a number of soluble factors, including the complement system and the chemokine/cytokine system; and a number of specialized cell types including mast cells, macrophages, dendritic cells (DCs), and natural killer cells. In contrast, the adaptive immune arm involves a delayed and a longer lasting antibody response together with CD8⁺ and CD4⁺ T cell responses that play a critical role in immunological memory against an antigen. A third arm of the immune system may be identified as involving γδ T cells and T cells with limited T cell receptor repertoires such as NKT cells and MAIT cells.

For an effective immune response to an antigen, antigen presenting cells (APCs) must process and display the antigen in a proper MHC context to a T cell, which then will result in stimulation of cytotoxic T cells and helper T cells. Following antigen presentation, successful interaction of co-stimulatory molecules on both APCs and T cells must occur or activation will be aborted. GM-CSF and IL-12 serve as effective pro-inflammatory molecules in many tumor models. For example, GM-CSF induces myeloid precursor cells to proliferate and differentiate into dendritic cells (DCs) although additional signals are necessary to activate their maturation to effective antigen-presenting cells necessary for activation of T cells. Barriers to effective immune therapies include tolerance to the targeted antigen that can limit induction of cytotoxic CD8⁺ T cells of appropriate magnitude and function, poor trafficking of the generated T cells to sites of malignant cells, and poor persistence of the induced T cell response.

DCs that phagocytose tumor-cell debris process the material for major histocompatibility complex (MHC) presentation, upregulate expression of costimulatory molecules, and migrate to regional lymph nodes to stimulate tumor-specific lymphocytes. This pathway results in the proliferation and activation of CD4⁺ and CD8⁺ T cells that react to tumor-associated antigens. Such cells can be detected frequently in the blood, lymphoid tissues, and malignant lesions of cancer patients. Compounds which are capable of stimulating an innate immune response as well as simultaneously priming an adaptive immune response may be particularly useful as immunotherapies for treating cancer.

### Immunotherapy

According to some aspects of the disclosure, the compositions described herein are administered with an amount effective of an immunomodulatory payload to stimulate (e.g., induce, increase or enhance) an immune response. An “immune response” may generally refer to responses that induce, increase, or perpetuate the activation or efficiency of innate or adaptive immunity. The compositions may be used functionally as adjuvants. The compositions may or may not be administered together with other adjuvants. Furthermore, the immune response may be enhanced relative to delivery of the payload (e.g., an immunomodulatory compound) alone and/or using a delivery vehicle other than the microbubble compositions described herein.

Specific stimulations of the immune response may comprise reducing inactivation and/or prolonging activation of T cells (e.g., increasing antigen-specific proliferation of T cells, enhancing cytokine production by T cells, stimulating differentiation and effector functions of cells, and/or promoting T cell survival) or overcoming cell exhaustion and/or anergy. According to certain embodiments, the composition may be effective to induce or increase the activation of STING. When used to stimulate an immune response, the compositions described herein may increase the number of immune cells producing proinflammatory cytokines, such as IFN-α, IFN-β, and/or IFN-γ, and/or increase the production of proinflammatory cytokines in existing immune cells. The increases may be detectable in a subject's serum. When used to stimulate an immune response, the compositions described herein may result in nuclear localization of pIRF3 and/or NFκ-B in treated cells. When used to stimulate an immune response, the compositions described herein may recruit CD8⁺ and/or CD4⁺ T cells to a site of treatment, infection, and/or cancer (e.g., to a tumor). A stimulated immune response may result in an increase number of effector memory T-cells and/or central memory T-cells (e.g., within a site of treatment, infection, or cancer) that are specific to an antigen. The antigen may be administered with or as a part of the microbubble composition or be an antigen present within a treatment area (e.g., a tumor antigen). A stimulated immune response may comprise an improved B-memory cell response (e.g., an increased frequency of peripheral blood B lymphocytes capable of differentiation into antibody-secreting plasma cells upon antigen encounter as measured by stimulation of in vitro differentiation).

An enhancement of an immune response may be quantified. According to certain aspects, the enhancement may be, for instance, at least about 10%, 20%, 30%, 40%, 50%, 60%, 70%, 80%, 90%, 100%, 150%, 200%, 300%, 400%, 500%, or 1,000% improved (e.g., increased) over a suitable baseline measurement. A suitable baseline measurement may be, for example, measured in a subject (e.g., prior to treatment) or a suitable reference population. The improvement may be statistically significant (e.g., p<0.05).

### Microbubble Compositions for Drug Delivery

According to various aspects of the disclosure, described herein are microbubble compositions which may be useful for drug delivery, according to the methods described elsewhere herein.

### Microbubbles

As used herein, “microbubble” (“MB”) may refer to a gas-filled bubble formed by a surfactant shell encapsulating a gas core. The surfactant shell may comprise one or more types of molecules which lower the interfacial tension between the gas core and the exterior aqueous environment, such as a physiological environment. This exterior shell may comprise, for example, lipids (e.g., phospholipids), proteins (e.g., albumin), sugars, and/or polymers. According to certain aspects, the gas cores comprise a perfluorocarbon (e.g., decafluorobutane). According to certain aspects, the microbubble shells comprise 1,2-Distearoyl-sn-Glycero-3-Phosphocholine (DSPC) and/or 1,2-Distearoyl-sn-Glycero-3-Phosphoethanolamine (DSPE) lipids. According to certain aspects, one or more of the components of the microbubble shell (e.g., the phospholipids) may be PEGylated. For instance, PEG chains may extend outward from the external surface of the microbubble. Various microbubble formulations are well known in the art. Microbubbles may be produced, for example, without limitation by any of the methods disclosed in U.S. Pat. No. 6,113,919 to Reiss et al. (issued Sep. 5, 200), 10,912,848 to Kim et al. (issued Feb. 9, 2021); or U.S. Patent Application Publication Nos. US 2002/0150539 to Unger (Oct. 17, 2002), US 2013/0336891 to Dayton et al. (published Dec. 19, 2013), or US 2018/0272012 to de Gracia Lux et al. (published Sep. 27, 2018); or Zhou, J Healthc Eng. 2013; 4(2):223-54 (doi: 10.1260/2040-2295.4.2.223), each of which is hereby incorporated by reference in its entirety.

In some embodiments, the bubble may be no greater than about 10 μm in diameter. In some embodiments, the average microbubble size within a microbubble composition is at least about 1, 2, 3, 4, or 5 μm. In some embodiments, the average microbubble size is approximately 1, 2, 3, 4, or 5 μm. In some embodiments, the average microbubble size is between approximately 1-10, 1-5, 1-4, 1-3, 1-2, 2-5, 2-4, 2-3, 3-5, or 3-4 μm.

Unless otherwise specified, microbubbles, as used herein, may comprise bubbles less than 1 μm (i.e. nanobubbles), such as bubbles between, for example, about 50 nm-100 nm, about 50 nm-200 nm, about 50 nm-300 nm, about 100 nm-200 nm, about 100 nm-300 nm, about 100 nm-400 nm, about 100 nm-500 nm, about 100 nm-1 μm, about 200 nm-1 μm, about 300 nm-1 μm, or about 500 nm-1 μm. In certain embodiments, the average size of the microbubble composition is between about 100 nm-700 nm, about 200 nm-600 nm, or about 300 nm-500 nm. The size of the microbubble composition (e.g., the average size or the maximum size) may be such that the microbubbles (or at least a majority of the microbubbles, e.g., at least 60%, 70%, 80%, 90%, 95%, 99% of the microbubbles) are able to extravasate the blood vessels of a subject, which may advantageously allow for systemic delivery of the microbubble composition in certain applications.

According to certain aspects, the microbubble composition may be a nanobubble composition. As used herein, a “nanobubble composition” may refer to a composition of microbubbles in which consists entirely of nanobubbles or is composed primarily of nanobubbles (e.g., at least about 60%, 70%, 80%, 90%, 95%, 99% of the microbubbles are nanobubbles). A nanobubble composition may be microbubble composition that is prepared in a manner to preferentially isolate nanobubbles or nanobubbles of a certain size. A nanobubble composition may be prepared, for example, by differential centrifugation, as described elsewhere herein. A nanobubble composition may have an average bubble size less than 1 μm. According to certain aspects, nanobubble compositions may be used for systemic delivery of microbubbles to a subject.

According to certain aspects of the disclosure, various payloads, such as CDNs, may be loaded onto the microbubble compositions described herein. As used herein, “loading” may refer to the binding of payload molecules to microbubbles. According to some aspects of the disclosure, payloads may preferably be reversibly bound to microbubbles though non-covalent interactions (e.g., electrostatic interactions). According to some aspects of the disclosure, payloads may preferably be bound to an external surface of the microbubbles (i.e. the microbubbles may be decorated with the payload). The microbubble compositions may incorporate one or more types of payload-binding molecules for loading the payload onto the microbubbles. The payload-binding molecules may be polymers. According to some preferred aspects of the disclosure, the payload-binding molecules may be cationic (e.g., cationic polymers). For example, the payload-binding molecules may comprise polyamines. According to some preferred aspects of the disclosure, the polyamines may comprise spermines, as described in further detail elsewhere herein.

According to various aspects of the disclosure, loaded microbubble compositions (compositions of microbubbles loaded with/bound to payloads) may be prepared in various ways. In some instances, the payload-binding molecules (e.g., cationic polymers) may first be associated with (e.g., conjugated to the microbubble surface), after which the microbubbles may be loaded with payload (e.g., by mixing or incubating with a payload solution). For example, spermine decorated microbubbles (e.g., SpeDex-decorated microbubbles) may be mixed with a solution of CDNs (e.g., cGAMP). In some instances, the payload-binding molecules may first be loaded with payload (e.g., via mixing) to form nanocomplexes, after which the resulting nanocomplexes may be conjugated to the microbubbles. For example, a solution of CDNs (e.g., cGAMP) may be mixed with a solution of spermine or SpeDex to form nanocomplexes, after which the nanocomplexes may be conjugated to microbubble surfaces (e.g., via free amine groups on the spermine). Loading active agents onto microbubbles may provide improved therapeutic efficacy relative to administering the active agent alone or in combination with the same microbubble composition but not bound to the microbubbles (e.g., with respect to the same microbubble compositions and the same amount of payload, loaded microbubble compositions may demonstrate increased intracellular delivery of the active agent after sonoporation relative to unbound combinations of the active agent and microbubble composition).

According to particular aspects of the disclosure, the microbubbles are decorated with spermines, which are particularly effective in non-covalently binding negatively charged payloads. According to more particular aspects of the present disclosure, the spermines are interlinked by dextrans, which allow for effective loading of spermines and negatively charged payloads to the microbubble surface. See e.g., PCT/US2021/054820 to Lux et al., filed Oct. 13, 2021, which is herein incorporated by reference in its entirety. Spermine-modified dextran (SpeDex) is a non-toxic cationic branched biopolymer that allows high loading of negatively charged payloads. According to specific aspects of the disclosure, SpeDex-decorated microbubbles are loaded with CDNs, such as cGAMP, for delivery to target cells, preferably via sonoporation. Strong electrostatic interaction between negatively charged CDNs and the cationic SpeDex polymers allows for efficient and stable binding. The high loading capacity of such microbubble compositions for suitable payloads, such as CDNs, may allow for improved drug delivery (e.g., larger amounts of payload being delivered intracellularly to target cells in each administration).

The payload may be any active agent which is able to be loaded onto a microbubble and preferably those that are suitable for intracellular delivery via sonoporation. The microbubble compositions described herein may be particularly useful for payloads which would benefit from targeted drug delivery. Payloads which stably bind spermine or SpeDex may, in particular, be suitable for drug delivery using the methods and/or compositions described herein, although other payload binding molecules (e.g., other cationic polymers such as polyamines) may be used to load microbubbles. According to certain aspects of the disclosure, the payload may bind to the microbubbles via electrostatic interactions between positive charges on the microbubble surface (e.g., positively charged primary and/or secondary amino groups) and negative charges exposed on the payload molecules, such as negatively charged phosphate groups on the sugar phosphate backbone of a nucleic acid or the phosphate group of a CDN. Depending on the payload, the microbubble compositions described herein may be loaded according to the ratio of the number of positively-chargeable amine groups (N) decorating the microbubbles of the microbubble composition to the number of negatively-charged phosphate groups (P) within the payload composition. In some embodiments, the microbubble composition may be loaded at N:P ratios of approximately 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the microbubble composition may be loaded at N:P ratios in which the number of phosphate groups (P) are at least about 5, 10, 15, or 20 times greater than the number of amine groups (N). In some embodiments, the microbubble composition may be loaded at N:P ratios of approximately 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the microbubble composition may be loaded at N:P ratios in which the number of amine groups (N) are at least about 5, 10, 15, or 20 times greater than the number of phosphate groups (P). In some embodiments, the microbubble composition may be prepared at concentrations of approximately 1×10⁻⁷-1×10⁻¹⁰, 1×10⁻⁷-1×10⁻⁹, 1×10⁻⁷-1×10⁻⁸, 1×10⁻⁸-1×10⁻¹⁰, or 1×10⁻⁸-1×10⁻⁹ μg of payload (e.g., pDNA) per microbubble. The microbubble composition may, for example, be prepared at concentrations of at least about 1×10⁻⁸, 2×10⁻⁸, 3×10⁻⁸, 4×10⁻⁸, 5×10⁻⁸, 6×10⁻⁸, 7×10⁻⁸, 8×10⁻⁸, 9×10⁻⁸ μg/microbubble. The amount of payload that a microbubble composition is ultimately able to carry may depends in part on the amount of positive charges available (e.g., the surface density of the amino groups on the microbubble surface) as well as the ability of the positively charged polymer to stably bind the nucleic acid. The binding stability of a microbubble composition may generally be reduced at higher loading capacities.

### Targeting Molecules

The microbubbles may be decorated with targeting molecules, which bind to specific cell types or other biological structures. In some embodiments, the targeting molecule may be a protein or other biomolecule (e.g., a ligand for a cell surface receptor). In some embodiments, the targeting molecule may be a biopolymer or component thereof, such as an extracellular matrix polymer (e.g., hyaluronic acid, collagen, elastin, fibronectin, laminin, or proteoglycans such as heparan sulfate, chondroitin sulfate, keratin sulfate). In some embodiments, the targeting molecule is a monoclonal or polyclonal antibody, including antibody fragments or peptides derived from/modeled after antibodies with antigen-binding properties. For instance, the antibody may be a Fab fragment, an F(ab′)2 fragment, an Fab′ fragment, an Fv fragment, an scFv, a di-scFv, an sdAb, a recombinant IgG, a peptide comprising one or more complementary determining regions (CDRs), or any other antibody fragment or biomolecule with antigen binding properties well known in the art. The antibody may be specific for an antigen expressed on the cell surface of the targeted cell type (e.g., a cell surface receptor). In some embodiments, the microbubbles may be configured to target cancerous/tumor cells. In some embodiments, the microbubbles may be configured to target immune cells (e.g., T-cells, B, cells, neutrophils, eosinophils, basophils, mast cells, monocytes, macrophages, dendritic cells, natural killer cells, etc.). According to certain aspects of the disclosure, microbubble compositions may comprise targeting molecules which bind CD11b (e.g., anti-CD11b antibodies). Cluster of differentiation molecule 11B (CD11b), also known as integrin alpha M (ITGAM) or CR3A, is one protein subunit that forms heterodimeric integrin alpha-M beta-2 (αMβ2) molecule, also known as macrophage-1 antigen (Mac-1) or complement receptor 3 (CR3). αMβ2 is expressed on the surface of many leukocytes involved in the innate immune system, including monocytes, granulocytes, macrophages, natural killer cells, and dendritic cells. According to some aspects of the disclosure, APCs, such as macrophages and dendritic cells, may be targeted for drug delivery by targeting CD11b.

The targeting molecules may be covalently coupled to the external surface of the surfactant shell of the microbubble. Various conjugation strategies are well known in the art for conjugating targeting molecules to microbubbles. See, e.g., PCT/US2021/054820 to Lux et al., filed Oct. 13, 2021, which is herein incorporated by reference in its entirety. Microbubble compositions comprising targeting molecules may provide improved therapeutic efficacy relative to microbubble compositions not targeted to any cell type (e.g., with respect to the same microbubble compositions loaded with the same amount of payload, targeted microbubble compositions may demonstrate increased intracellular delivery of the payload after sonoporation even when using localized administration, such as after intratumoral injection).

### Payloads

The microbubble compositions described herein may be loaded with one or more “payloads” or “active agents.” The terms “payload” or “active agent” or “drug,” as used herein, refer to any compound used for the treatment or diagnosis of a disease. Drug delivery may refer to the delivery of a payload or active agent to a cell or subject, preferably to a target organ, tissue, and/or cell type. A microbubble composition as used herein may facilitate drug delivery of the one or more active agents to a target organ, tissue, or cell.

Exemplary active agents include, but are not limited to, compounds that rely on intracellular access and/or compounds that rely on access to cytosolic receptors/pathways, including STING agonists, such as cyclic dinucleotides (CDNs), as described in further detail herein. The present disclosure, however, contemplates additional or alternative payloads besides CDNs.

Additional representative suitable active agents include, but are not limited to, STING antagonists, oligonucleotides, proteins, peptides, peptides, lipopeptides, polysaccharides, hydrophobic and amphiphilic small molecular drugs, antibodies, nanobodies, RNA, mRNA, miRNA, siRNA, aptamers, antibiotics, antigens (e.g., tumor antigens, tumor neoantigens), chemotherapeutics, imaging agents, quantum dots, any other suitable compound for disease treatment, or a combination thereof. According to certain aspects of the disclosure, the payload may comprise a nucleic acid. The nucleic acid may be, for example, a plasmid (e.g., pDNA), an siRNA, or a Dicer-substrate siRNA (DsiRNA). The nucleic acid may be DNA, RNA, or combinations thereof. For example, the methods and/or compositions described herein may be used for targeted gene delivery (e.g., siRNA, mRNA, DNA, etc.), such as for gene therapy. According to certain aspects of the disclosure, the payload may comprise a protein. Protein therapeutics may comprise, for example, antibody-based drugs, anticoagulants, blood factors, bone morphogenetic proteins, engineered protein scaffolds, enzymes, Fc fusion proteins, growth factors, hormones, interferons, interleukins, thrombolytics, etc. Representative examples of therapeutic applications include, but are not limited to, delivery of immunostimulatory DNA to immune cells; cytosolic delivery of antigens to dendritic cells to increase antigen presentation on class I MHC, cytosolic delivery of vaccine antigen/adjuvant combinations and intracellular cytokine staining.

Different types of payloads may be loaded onto the same microbubble (e.g., immunomodulatory compounds and antigens). Microbubble compositions may comprise mixtures of microbubbles having different payloads or payload profiles. According to some aspects of the disclosure, a microbubble composition may comprise two or more of the active agents described herein, including the agents discussed with respect to combination therapies (e.g., a CDN and an anti-cancer agent). The microbubble compositions may have one or more types of targeting molecules and/or microbubble compositions may comprise mixtures of microbubbles having different targeting molecules or targeting molecule profiles.

### STING Signaling and STING Agonists

As described elsewhere herein, the methods and/or compositions of the present disclosure may be particularly useful for delivery of CDNs, particularly to immune cells, including both macrophages and dendritic cells. Specifically, the methods and/or compositions of the present disclosure may be particularly useful for delivery of STING agonists, preferably intracellular delivery of STING agonists to target cells. An “agonist,” as it relates to a ligand and receptor, comprises a molecule, combination of molecules, a complex, or a combination of reagents, that stimulates the receptor. For example, a STING agonist can encompass cGAMP, a mutein or derivative of cGAMP, a peptide mimetic of cGAMP, a small molecule that mimics the biological function of cGAMP, or an antibody that stimulates STING. Accordingly, preferred payloads may be cGAMP or other STING agonists. According to some aspects of the disclosure, CDNs may be delivered to cancer cells.

Stimulator of interferon genes (STING), also known as transmembrane protein 173 (TMEM173) and MPYS/MITA/ERIS, is an adaptor protein in the cytoplasm of mammalian cells which activates the TANK binding kinase (TBK1)—interferon regulatory factor 3 (IRF3) signaling axis via a phosphorylation-dependent mechanism, resulting in the induction of IFN-β and other IRF-3 dependent gene products that strongly activate innate immunity. TKB1 is a serine/threonine protein kinase which regulates cell proliferation, apoptosis, autophage, and anti-tumor immunity. TKB1 phosphorylates and activates IRF3. IRF3 is an inteferon regulatory factor (transcription factor). Sting also activates the STAT6 transcription factor, which mediates signaling required for the development of T-helper type 2 (Th2) cells and Th2 immune response. STAT6 and IRF3 are responsible for antiviral response and innate immune response against intracellular pathogens. STING, thus, plays an important role in innate immunity. STING also activates NF-κB, a protein complex that controls transcription of DNA, cytokine production and cell survival, and which functions together with IRF3 to turn on the transcription of type I interferons (IFNs) and other cytokines. STING induces type I interferon production when cells are infected with intracellular pathogens, such as viruses, mycobacteria and intracellular parasites. Type I interferons play important roles in both the adaptive and innate immune responses, prevent proliferation of pathogens, and have antiviral activities. Type I interferons, mediated by STING, protect nearby cells from local infection via paracrine signaling. STING is encoded in humans by the STING1 gene (Gene ID: 340061).

STING is considered a pathogen recognition receptor (PRR) which functions as a direct cytosolic DNA sensor (CDS) and is a component of the host cytosolic surveillance pathway. The pathway senses infection with intracellular pathogens and in response induces the production of IFN-β, leading to the development of an adaptive protective pathogen-specific immune response consisting of both antigen-specific CD4⁺ and CD8⁺ T cells as well as pathogen-specific antibodies. As described herein, the STING signaling pathway in immune cells is a central mediator of innate immune response and when stimulated, induces expression of various interferons, cytokines and T cell recruitment factors that amplify and strengthen immune activity, including against infections and cancerous cells. The STING signaling pathway is described in further detail in Li et al., J Exp Med. 2018 May 7; 215(5):1287-1299 (doi: 10.1084/jem.20180139) and Zhu et al., Mol Cancer. 2019 Nov. 4; 18(1):152 (doi: 10.1186/s12943-019-1087-y), each of which is hereby incorporated by reference in its entirety.

Cyclic dinucleotides (CDNs), such as cyclic guanosine monophosphate-adenosine monophosphate (cGAMP), are agonists of STING and have potential therapeutic applications, particularly in oncology and immunology. The cyclic dinucleotides recognized by STING are small-molecule second messengers used by all phyla of bacteria and are also produced as endogenous products of the cytosolic DNA sensor cyclic GMP-AMP synthase. Thus, such CDNs may be considered pathogen associated molecular patterns (PAMPs). The CDNs cyclic-di-AMP (produced by Listeria monocytogenes) and its analog cyclic-di-GMP (produced by Legionella pneumophila) are also agonists of STING. A number of other STING agonists have been discovered, or developed, in attempt to either treat tumors alone, or to be used in combination with other cancer therapies to enhance their performance. See, e.g., La Nour et al. Oncoimmunology. 2020 Jun. 16; 9(1):1777624, which is herein incorporated by reference in its entirety. These include flavone acetic acid (FAA) and 5,6-dimethylxanthenone-4-acetic acid (DMXAA). Examples of cyclic purine dinucleotides that may bind STING and/or which may be delivered via the compositions described herein are described in some detail in, e.g., U.S. Pat. Nos. 7,709,458 and 7,592,326; WO2007/054279; and Yan et al., Bioorg. Med. Chem Lett. 18: 5631 (2008), each of which is hereby incorporated by reference in its entirety.

Cyclic dinucleotides are difficult to deliver intracellularly. As described herein, STING agonists may be delivered directly into the cytoplasm of cells via the application of ultrasound to microbubble compositions loaded with the STING agonist. This delivery platform may be used as an immunotherapy for cancer, which in that context, is referred to as Microbubble-assisted Ultrasound-guided Immunotherapy of Cancer (abbreviated as MUSIC herein). Microbubbles allow the targeted delivery of STING agonists, such as cGAMP, to target cells (e.g., cancer cells or immune cells) and ultrasound-induced sonoporation can be used to deliver the STING agonist payloads across cell membranes in an endocytosis-independent manner.

### Vaccines

According to some aspects of the disclosure, the compositions described herein are administered as immunogenic compositions or prophylactic vaccines which confer resistance in a subject to subsequent exposure to infectious agents, or as part of therapeutic vaccines, which can be used to initiate or enhance a subject's immune response to a pre-existing antigen, such as a viral antigen in a subject infected with a virus or with cancer. Immunogenic compositions may comprise immunomodulatory compounds which stimulate a desired immune response, as described elsewhere herein. The microbubbles may be loaded with immunomodulatory compounds, such as CDNs (e.g., cGAMP); antigens (e.g., protein or peptide antigens); or combinations thereof. Research has shown CDNs promote cellular and humoral immunity in vaccinated mice. Accordingly, CDN-based vaccine adjuvants may be used to improve vaccine efficacy. The use of the methods and/or compositions described herein to deliver immunomodulatory compounds, antigens, or combinations thereof may provide for increased antigen delivery and/or increased antigen presentation. According to some aspects, a vaccine is formed by a combination of the immunomodulatory compound and antigen, wherein at least one or both the immunomodulatory compound and antigen are loaded onto a microbubble composition of the present disclosure. According to some aspects, one of the vaccine components (e.g., the antigen) may be delivered via a different type of composition (e.g., using a vehicle other than a microbubble composition or no vehicle at all). The immunomodulatory compound and antigen may be delivered together to act as a vaccine, whether they are part of a single pharmaceutical composition or not.

Antigens can be, without limitation, peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. The antigens may be whole inactivated or attenuated organisms. These organisms may be infectious organisms, such as viruses, parasites and bacteria. These antigen may be a tumor cell. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA. Antigens may be provided as single antigens or may be provided in combination. Antigens may be provided as complex mixtures of polypeptides or nucleic acids. Antigens may be allergens or environmental antigens. Antigens may be cancer antigens (an antigen that is typically expressed at higher levels in cancer cells than on non-cancer cells or is expressed solely by cancer cells).

According to some aspects of the disclosure, the microbubble compositions described herein may be used as part of a therapeutic vaccine. As used herein, a “therapeutic vaccine” may refer to a vaccine which is administered after an infection or disease, such as cancer, has already affected a subject. A therapeutic vaccine may activate the immune system of a subject to fight an existing infection or disease. A therapeutic vaccine may help, for example, a subject's immune system to recognize and respond to a cancerous cells. The microbubble composition may be delivered to a subject as a therapeutic vaccine which targets an existing cancer via targeted sonoporation of a cancer site (e.g., a tumor). According to some aspects, the drug-loaded microbubble composition may be used to activate APCs, such as dendritic cells, ex vivo. APCs may be extracted from a subject via leukapheresis and activated ex vivo via sonoporation of drug-loaded microbubbles (e.g., loaded with a STING agonist, such as a CDN) as well as incubation with a cancer cell antigen or cancerous cells (e.g., isolated from a patient biopsy) before being reintroduced to the subject as a vaccine (e.g., a dendritic vaccine). Activation of the APCs may comprise treatment/incubation with other agents, such as granulocyte-macrophage colony stimulating factor (GM-CSF), for example.

### Methods of Treatment

The methods of treatment described herein comprise administration of any of the drug-loaded microbubble compositions described elsewhere herein, preferably for intracellular delivery of the drug/payload to a target organ, tissue, or cell, including, for example, the intracellular delivery of immunomodulatory payloads (e.g., CDNs such as cGAMP), such as for the treatment of cancer and/or as part of vaccines, and genes for gene therapy.

### Administration of Drug Loaded Microbubble Compositions

The methods and/or compositions described herein may be used to treat one or more various diseases. As used herein, a “disease,” “disorder,” “condition,” “illness,” “ailment,” or “indication” may be used interchangeably and may refer to any physiological state or pathology of a subject which may reasonably be treatable by the methods and/or compositions described herein. A disease may be caused by one or more contributing factors, including, for example, genetic factors (i.e. specific genotypes), epigenetic factors, behavioral/lifestyle factors (e.g., diet and exercise), age, and external factors (e.g., toxin exposures, infections, injuries, etc.), some of which may be interrelated. A disease may or may not have a definitive etiology. A disease may be associated with one or more symptoms. A disease may be a syndrome classified by one more symptoms. A disease may be clinically diagnosable by one or more well-known means in the art, including, for example, measurable clinical parameters (e.g., blood work, urinalysis, measurement of biomarkers); functional assessments; physical examinations; diagnostic imaging; histological analysis (e.g., biopsies); genotyping/genetic sequencing; evaluation of subject medical history; etc.

“Treatment” of a disease or “treating” a disease, as used herein, may refer to an approach of applying the methods and/or compositions described herein for obtaining beneficial or desired results (a therapeutic response), including clinical results. “Therapy,” as used herein, may be used interchangeably with “treatment.” Beneficial or desired results with respect to treating a subject for a disease may include, but are not limited to, one or more of preventing a disease, delaying the onset of a disease, curing or resolving a disease, lessening the severity of a disease, delaying progression of a disease, preventing the worsening of a disease, increasing the quality of life of one suffering from a disease, prolonging survival of a subject having a disease, and/or improving the therapeutic efficacy of other treatments for the disease (e.g., reducing the dose necessary to achieve a therapeutic response). Such beneficial or desired results may be achieved with respect to any aspect of a disease pathology or comorbidity associated therewith, including physiological states or processes that cause or contribute to a disease as well as effected physiological states or processes that manifest in symptoms or complications associated with the disease. Thus, for example, lessening the severity of a disease may encompass alleviating (e.g., reducing the severity, frequency, or duration of or eliminating altogether) one or more symptoms associated with a disease or comorbidity thereof. Assessments of beneficial or desired results may comprise assessments of any one or more factors that could be used, at least in part, to diagnose a disease or comorbidity thereof (e.g., assessing for discernible improvements in such a factor). According to some aspects of the disclosure, the beneficial or desired results may be assessed relative to a population having the same risk factors, symptoms, and/or type and severity of disease (e.g. as assessed by clinical measurements) and not receiving treatment as described herein. Depending on the context, “treatment” of a subject can imply that the subject is in need of treatment, e.g., in a situation where the subject has been diagnosed with or exhibits symptoms or complications associated with a disease reasonably expected to be treatable with a method and/or composition described herein. “Treatment,” as used herein also encompasses prophylactic treatments. As used herein, “prevention” or “preventing,” when used in reference to a disease, includes a reduction in likelihood of developing a disease (e.g., reducing or improving on risk factors for a disease) and/or a reduction in severity of a disease upon onset. In cases of prophylactic treatment, a subject may be in need of treatment (e.g., for preventing further development or progression of a disease) or determined to be at a relatively high risk of disease (e.g., such that prospective benefits of prophylactic treatment outweigh any risks, such as side effects, associated therewith).

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that can be treated with the methods and/or compositions of the present disclosure. Non-human subjects may be livestock. According to some specific aspects of the disclosure, the subject is a human, such as a human having cancer. The subject may be a female (e.g., a female human). The subject may be a male (e.g., a male human). In some aspects, the subject is an adult subject. In other aspects, the subject is a pediatric subject, such as a neonate, an infant, or a child. The subject may be an individual for which it is reasonably expected a therapeutic response could be achieved. Subjects may be individuals who are administered a composition of the present disclosure for experimental or research purposes (e.g., control subjects). A “patient,” as used herein, refers to a subject who exhibits symptoms and/or complications of a disease; has been diagnosed as having a disease; has been identified as being at a risk of developing a disease; and/or is under the treatment of a clinician (e.g., a physician), including for investigation of some pathology that could be associated with the disease, even if no defined disease has been diagnosed. The term “patient” includes human and veterinary subjects. Any reference to subjects in the present disclosure, should be understood to include the possibility that the subject is a “patient” unless indicated otherwise, explicitly or by context.

According to certain aspects of the disclosure, the microbubble compositions described herein may be incorporated into a pharmaceutical composition suitable for administration to a subject. Such pharmaceutical compositions may further comprise a “pharmaceutically acceptable carrier,” (interchangeable with “pharmaceutical carrier” or “carrier”) which may be any compound or composition useful in facilitating storage, stability, administration, cell targeting and/or delivery of the microbubbles to a target cell or cell population. A “pharmaceutically acceptable carrier,” as used herein, refers to a carrier or excipient that is suitable for use with the subjects or patients described elsewhere herein (e.g., humans and/or animals) without undue adverse side effects (such as toxicity, irritation, and allergic response) commensurate with a reasonable benefit/risk ratio. The carrier can be a pharmaceutically acceptable solvent, dispersion media, suspending agent or other suitable vehicle, for delivering microbubble compositions as described herein to the subject, such as through, for example, intravenous injection. Pharmaceutically acceptable carriers may include any diluents, solvents (including water), fillers, extenders, preservatives, thickeners, antibacterial agents, antifungal agents, isotonic agents, pH modifiers, salts, colorants, flavorings, rheology modifiers, lubricants absorption delaying agents, antifoaming agents, surfactants, emulsifiers, adjuvants, suitable vehicles, coatings, erodible polymers, hydrogels, phospholipids, fatty acids, mono-di- and tri-glycerides and derivates thereof, waxes, oils, etc., which are compatible with pharmaceutical administration. The use of such media and agents for pharmaceutically active substances is well known in the art. Except insofar as any conventional media or agent is incompatible with the microbubbles and/or components thereof (e.g., payloads, targeting molecules), use thereof in the compositions is contemplated. Supplementary active agents can also be incorporated into the pharmaceutical compositions. A pharmaceutical composition according to the disclosure may be formulated to be compatible with its intended route of administration, as described elsewhere herein. The pharmaceutical composition can be included in a kit, container, pack, or dispenser together with instructions for administration.

Treatment of a subject, organ, tissue, cell, or body fluid with a composition described herein, generally comprises administering the composition to a subject, organ, tissue, cell, or body fluid, respectively. “Administration,” as used herein, refers to inducement of contact between an exogenous compound, such as the microbubble compositions described herein and/or payloads loaded thereon, a placebo, or a control, to a subject, organ, tissue, cell, or body fluid. Treatment of a cell, for example, encompasses contact of a compound to the cell, as well as contact of a compound to a fluid, where the fluid is in contact or placed into contact with the cell. Administration may be performed for therapeutic or experimental (e.g., basic research, clinical research, pharmacokinetic studies) purposes. When multiple compounds are “administered together” they are not necessarily administered as a single composition, although they may be. Such compounds may be delivered to a single subject as separate administrations, which may be at the same or different time, and which may be by the same route or different routes of administration, unless dictated otherwise explicitly or by context.

The microbubbles may be administered locally, regionally or systemically as desired, for example and without limitation: intravenously, intramuscularly, subcutaneously, dermally, subdermally, intraperitoneally, transdermally, iontophoretically, orally, and transmucosally. Non-limiting examples of devices useful in delivering the microbubbles to a subject include needle/syringes, catheters, trocars, stents or projectiles. According to certain aspects of the disclosure, the microbubbles are delivered directly to a cite in need of an immune response. For example, the microbubbles may be delivered intratumorally, which includes delivery internal to a tumor and/or immediately adjacent to a tumor or a cancer cell such that the decoy diffuses to contact the tumor or cancer cell. According to certain aspects of the disclosure, the microbubbles are administered at a site adjacent to or leading to one or more lymph nodes which are close to the site in need of an immune response (e.g., a tumor).

Treatments, as described herein, may comprise one or more rounds of administration (administration of one or more doses) of a composition described herein. A “treatment regimen” or simply “regimen,” as used herein may refer to a course of treatment comprising multiple administrations of a composition described herein. The composition may be the same for each round of administration (or for at least some of the rounds of administration) or may be different for at least some rounds of administration. The doses may be the same or different for different rounds of administering the same composition. A treatment regimen may be defined by a dosing schedule (e.g., a dosing frequency or temporal pattern of administrations), a total number of administrations/doses, and/or a total duration in time of treatment. Some of these parameters may be determined independently, whereas some may be dependent on the other parameters or combinations thereof. Some parameters may be left indefinite for a particular regimen. Some parameters may be determined or adjusted as treatment progresses. For example, a clinician may adjust dosing amount and/or frequency based on observable responses to prior rounds of administration. According to some aspects of the disclosure, each round of administration may comprise administering the same composition at the same dose via the same route of administration. According to some aspects of the disclosure, a treatment regimen may comprise specific compositions (including combination therapies), doses, and/or routes of administration where any of these variables is not consistent at each round of administration.

Where there is more than one administration, the administrations can be spaced apart by time intervals of approximately one 1, 2, 3, 4, 5, 6, 7, 8, 9, 10, 15, 20, 30, or 45 minutes; by intervals of about 1, 2, 3, 4, 5, 6, 7, 8, 9, 10, 11, 12, 13, 14, 15, 16, 17, 18, 19, 20, 21, 22, 23, or 24 hours; by 1, 2, 3, 4, 5, 6, or 7 days; by 1, 2, 3, 4, or 5 weeks; or by 1, 2, 3, 4, 5, 6, 7, 8, 9, 10, 11, 12 or more months, including intervening time ranges. The doses may be administered at substantially regular time intervals (a frequency of administration). The administrations, however, are not limited to dosing intervals that are spaced equally in time, but may encompass doses at non-equal intervals as well. According to some aspects of the disclosure, a dosing frequency may be determined based at least in part on pharmacokinetic and/or pharmacodynamics profiles for the composition being administered as is well understood in the art. For instance, the frequency of administration may be tailored to generally maintain an administered composition, a component thereof (e.g., a payload), a metabolite thereof, or a biological response thereto (e.g., a marker of therapeutic efficacy) above a threshold (e.g., effective for inducing a therapeutic response), below a threshold (e.g., for avoiding or minimizing adverse effects, including toxicity concerns and side effects), or within a prescribed range.

Where there is more than one administration, the administrations may be spaced apart over a duration of treatment. The duration of the treatment regimen may be, for example, approximately or at least about 1, 2, 3, 4, or 5 weeks; 1, 2, 3, 4, 5, 6, 7, 8, 9, 10, 11, or 12 months; or 1, 2, 3, 4, or 5 years. A treatment regimen may comprise, by example, a total of about 2, 3, 4, 5, 6, 7, 8, 9, 10, 11, 12, 13, 14, 15, 16, 17, 18, 19 20, 21, 22, 23, 24, 25, 30, 35, 40, 45, 50, 55, 60, 65, 70, 75, 80, 85, 90, 95, 100 or more administrations. According to some aspects of the disclosure, the treatment may be continued along some dosing schedule (e.g., at a dosing frequency) at least until a therapeutic response is achieved or until a therapeutic response is shown to be likely maintained for a sufficient period of time absent further administrations.

“Therapeutically effective” or “effective,” as used herein, in reference to amounts, frequencies, durations, and regimens refers to the amount of a composition, frequency of administration, duration of treatment, or design of treatment regimen, respectively, which would be reasonably expected to be sufficient in achieving a therapeutic response, such as those benefits and results described elsewhere herein with respect to treatments. According to certain aspects of the disclosure, therapeutically effective may refer to an amount, frequency, duration, or regimen that is sufficient to achieve some statistically significant (e.g., p<0.05) measure of a therapeutic response. According to certain aspects of the disclosure, therapeutically effective may refer to an amount, frequency, duration, or regimen that is sufficient to achieve at least about a 5%, 10%, 15%, 20%, 25%, 30%, 40%, or 50% change in a baseline value (e.g., measured in a subject prior to treatment or measured in a suitable reference population). Means for statistically evaluating therapeutic efficacy are well known in the art. According to some aspects of the disclosure, therapeutic efficacy may be assessed by comparing outcomes in a population of subjects treated with a particular amount, frequency, duration, and/or regimen against outcomes to a population of similarly situated subjects not receiving treatment as described herein. Therapeutically effective amounts, frequencies, durations, and regimens of the administering the compositions described herein may vary according to factors such as the degree of susceptibility of the individual, the age, gender, and weight of the individual, and idiosyncratic responses of the individual. Unless dictated otherwise, explicitly or by context, a “therapeutically effective” amount, frequency, duration, and/or regimen is not limited to a minimal amount, frequency, and/or duration sufficient to achieve a therapeutic response or a regimen comprising such minimal amounts, frequencies, and/or durations. The therapeutic efficacy of the dosing amount, dosing frequency, and/or treatment duration may be interrelated, particularly depending on the therapeutic response used to evaluate efficacy. For instance, more sustained measures of therapeutic efficacy (e.g., curing a disease) are likely to require more rounds of administration than a more transient measure of therapeutic efficacy (e.g., alleviating a symptom of a disease).

Therapeutically effective amounts of a pharmaceutical composition, a microbubble composition within a pharmaceutical composition, and/or a payload of the microbubble composition may be interrelated. The effective amount of a microbubble composition may be determinable, for instance, based on a loading efficiency of the particular combination of microbubble composition and payload, as may be readily measured experimentally, as described, for example, elsewhere herein.

The dosage of microbubbles administered to a subject may depend on the route of administration. Data obtained from in vitro cell-based assays and/or animal studies can be used in formulating a range of dosages for use in subjects (e.g., humans). The dosage may lie within a range of circulating concentrations that includes the ED50 with little or no toxicity. The dosage may vary within this range depending upon the dosage form employed, the route of administration utilized, and the particular indication (e.g., cancer type) being treated. For instance, intratumoral injection may require smaller dosages than systemic administration routes. Intravenous, or intramuscular systemic delivery may require larger dosages.

An effective amount of payload (e.g., a CDN such as cGAMP) to be administered (e.g., systemically) may generally be between about 0.1 μg/kg-1,000 mg/kg, 1 μg/kg-1,000 mg/kg, 10 μg/kg-1,000 mg/kg, 100 μg/kg-1,000 mg/kg, 1 mg/kg-1,000 mg/kg, 0.1 μg/kg-100 mg/kg, 1 μg/kg-100 mg/kg, 10 μg/kg-100 mg/kg, 100 μg/kg-100 mg/kg, 1 mg/kg-100 mg/kg, 0.1 pig/kg-10 mg/kg, 1 jig/kg-10 mg/kg, 10 jig/kg-10 mg/kg, 100 jig/kg-10 mg/kg, 1 mg/kg-10 mg/kg, etc. The effective amount may depend upon a variety of factors including, for example, the activity of the specific payload employed, subject-specific factors (age, body weight, general health, sex and diet of the individual being treated), the time and route of administration, the rate of excretion, other treatments which have previously been administered, the precise condition being treated, the severity of the particular condition undergoing treatment, etc. The attending clinician may use appropriate discretion as is understood in the art on deciding upon an effective dose. Guidance for methods of treatment and diagnosis are well known in the art (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

### Sonoporation

The present disclosure provides methods of delivering payloads to one or more cells using the microbubbles described herein with sonoporation techniques. Methods for performing sonoporation are well known in the art. See, e.g., Chowdhury et al., Ultrasonography. 2017 July; 36(3):171-184 (doi: 10.14366/usg.17021); Tzu-Yin et al., Curr Pharm Biotechnol. 2013; 14(8):743-52 (doi: 10.2174/1389201014666131226114611); Xu et al., Adv. Therap., 4: 2100154. (doi.org/10.1002/adtp.202100154), each of which is hereby incorporated by reference in its entirety. Sonoporation uses sound, typically at ultrasonic frequencies, for increasing the permeability of the cell plasma membrane in order to facilitate the delivery of a payload across the membrane and into the cell. Sonoporation may be particularly beneficial for delivering payloads across the blood-brain barrier or blood-tumor barrier. For in vivo applications, sonoporation may be an advantageous means of drug delivery as ultrasound can penetrate deep into the subject's tissue in a non-invasive manner as well as provide spatially and temporally targeted delivery with minimal or no side effects to non-targeted tissue. Microbubbles can function as nuclei for acoustic cavitation in ultrasound-mediated drug delivery, effectively scattering ultrasound waves due to the high compressibility of the microbubbles. Sonoporation is believed to induce transient increases in cell permeability via the formation of transient pores in the cell plasma membrane. Without being bound by theory, collapsing microbubbles may produce local shock waves, water jets, and shear forces that are able to permeabilize nearby cell membranes. Sonoporation may allow the direct delivery (i.e. outside the endosomal transport pathway) of therapeutics, such as nucleic acids, into a cell's cytosol.

The intensity of the ultrasound waves and the composition of the microbubble shell may influence the effectiveness of the sonoporation. Microbubble shells should be stiff enough to withstand small pressure perturbations but elastic enough to oscillate in response to the ultrasound waves. Lower degrees of polydispersity in microbubble size may be desirable so that larger proportions of a microbubble composition are sensitive to the same amplitudes of ultrasound. Without being limited by theory, microbubbles subjected to low-intensity ultrasound may oscillate stably around a resonant diameter, termed stable cavitation. Stable cavitation generates local shear forces and acoustic microstreaming. At higher pressure amplitudes, microbubbles tend to undergo large size variations which cause them implode in an event termed inertial cavitation, the collapse resulting in water jetting, shock waves and other inertial phenomena. Both stable and transient microbubble cavitations may induce cell membrane permeabilization. Sonoporation is believed to provide a pathway to intracellular drug delivery independent of endocytosis.

According to certain aspects of the disclosure, ultrasound triggering of sonoporation with microbubbles may be performed at frequencies between about 0.1 MHz and about 10 MHz, between about 0.5 MHz and about 5 MHz, or between about 1 MHz and about 3 MHz. In some embodiments, ultrasound triggering may be performed at intensities between about 100 mW/cm² and about 1 kW/cm², between about 100 mW/cm² and about 1 W/cm², between about 300 mW/cm² and about 1 W/cm², between about 500 mW/cm² and about 1 W/cm², between about 700 mW/cm² and about 1 W/cm², between about 1 W/cm² and about 500 W/cm², between about 1 W/cm² and about 100 W/cm², between about 1 W/cm² and about 100 W/cm², between about 1 W/cm² and about 50 W/cm², between about 1 W/cm² and about 20 W/cm², between about 1 W/cm² and about 10 W/cm², between about 1 W/cm² and about 5 W/cm², between about 1 W/cm² and about 2 W/cm², and between 0.1 W/cm² and 1 W/cm². In some applications, the ultrasound triggering may be performed at a maximal intensity permitted by a regulatory agency (e.g., the FDA), such as, for example, approximately 720 mW/cm² (for diagnostic ultrasound). In some applications, the ultrasound triggering may be performed at a duty cycle between about 10% and 100%, between about 20% and about 90%, between about 30% and about 80%, between about 40% and about 70%, or between about 50% and about 60%. In some applications, the duty cycle is about 50%. In some applications, the mechanical index (MI) of the ultrasound may be between about 0.05 and about 5, between about 0.1 and about 5, between about 0.5 and about 5, between about 1 and about 5, between about 2 and about 5, between about 3 and about 5, between about 4 and about 5. In some applications, the ultrasound triggering may be performed at a maximal mechanical index permitted by a regulatory agency (e.g., the FDA), such as, for example, approximately 1.9 (for diagnostic ultrasound). In some applications, the ultrasound triggering may be delivered for about 10 s-3 min, 10 s-2 min, 10 s-min, 10 s-50s, 10 s-40 s, 10 s-30 s, 30 s-3 min, 30 s-2 min, 30 s-1 min, 30 s-50 s, 30 s-40 s, 1 min-3 min, or 1 min-2 min, 2 min-3 min. According to certain aspects of the disclosure, ultrasound may be used to visualize the microbubbles prior to sonoporation (e.g., to confirm targeted localization of the microbubbles). Such an ultrasound stimulus may be administered at parameters (e.g., an intensity) that are configured not to induce sonoporation.

### Treatment of Cancer

According to some aspects of the disclosure, the compositions and/or methods described herein may be used to treat a disease associated with abnormal apoptosis or an abnormal differentiative process, including cellular proliferative disorders (e.g., hyperproliferative disorders) and/or cellular differentiative disorders, such as cancer. Cancers may be treated, for example, with an immunomodulatory composition (e.g., comprising a CDN such as cGAMP) or a vaccine, as described elsewhere herein.

As used herein, the terms “cancer” (or “cancerous”), “hyperproliferative,” and “neoplastic” refer to cells having the capacity for autonomous growth (i.e., an abnormal state or condition characterized by rapidly proliferating cell growth). A “tumor” or “neoplasm” may be used interchangeably to refer to an abnormal growth of cells. Hyperproliferative and neoplastic disease states may be categorized as pathologic/malignant (i.e., characterizing or constituting a disease state), or categorized as non-pathologic/benign (i.e., as a deviation from normal but not associated with a disease state). These terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” as used herein encompass malignancies of the various organ systems, including those affecting the lung, breast, thyroid, lymph glands and lymphoid tissue, gastrointestinal organs, and the genitourinary tract, as well as to adenocarcinomas. The term “carcinoma” refers specifically to cancerous malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. Adenocarcinomas are generally considered to include cancerous malignancies such as most colon cancers, renal-cell carcinoma, prostate cancer and/or testicular tumors, non-small cell carcinoma of the lung, cancer of the small intestine and cancer of the esophagus. Additional examples of proliferative disorders include hematopoietic neoplastic disorders, which include diseases involving hyperplastic/neoplastic cells of hematopoietic origin (e.g., arising from myeloid, lymphoid or erythroid lineages, or precursor cells thereof).

The methods and/or combinations described herein may be particularly useful for generally treating neoplastic conditions in subjects which may include-benign or malignant tumors (e.g., adrenal, liver, kidney, bladder, breast, gastric, ovarian, colorectal, prostate, pancreatic, lung, thyroid, hepatic, cervical, endometrial, esophageal and uterine carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders. More specifically, neoplastic conditions subject to treatment in accordance with the methods and/or compositions described herein may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple, myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

Neoplastic conditions comprising solid tumors, in particular, may benefit from treatment according to the methods and/or compositions described herein, as localized masses of target cells may be efficiently targeted by sonoporation, although hematologic malignancies are also contemplated within the scope of the disclosure. Some lymphomas, in particular, may present as solid masses may be effectively targeted by the methods and/or compositions described herein. According to certain aspects of the disclosure, the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors.

The compositions and methods may be used to treat an existing tumor (e.g., primary tumor) in a subject by using sonoporation to target drug delivery to that tumor. Treatment may result in the improvement, reduction, or alleviation of one or more symptoms of cancer as is known in the art, including for example, tumor size, tumor volume, tumor growth, or metastasis. Such improvements may be quantified and may be, for instance, at least 10%, 20%, 30%, 40%, 50%, 60%, 70%, 80%, 90%, 100%, 150%, 200%, 300%, 400%, 500%, or 1,000% improved over baseline measurements as measured in a subject (e.g., prior to treatment) or a suitable reference population. The improvement may be statistically significant (e.g., p<0.05). Other suitable measures of treatment may include, for example, measurement of markers of an innate or adaptive immunity response (e.g., proinflammatory markers, T-cells, etc.). Such markers may be measured, for example, in tissue biopsies or cell suspension prepared therefrom or in blood (e.g., serum) levels. Changes in marker levels from treatment may be at least about 10%, 20%, 30%, 40%, 50%, 60%, 70%, 80%, 90%, 100%, 150%, 200%, 300%, 400%, 500%, or 1,000% over baseline measurements as measured in a subject (e.g., prior to treatment) or a suitable reference population. Changes may be statistically significant (e.g., p<0.05). According to some aspects of the disclosure, the treatment may be, at least in part, prophylactic. The treatment may, for example, comprise treatment of subjects who present with benign or precancerous tumors and/or prevent or reduce the likelihood of worsening of a cancer (e.g., growth of a tumor or metastatic spread of a tumor) or the recurrence of a cancer. A metastatic tumor can arise from a multitude of primary tumor types, including but not limited to those of prostate, colon, lung, breast and liver. The treatments described herein are also contemplated to encompass treatment of metastatic tumors.

By way of example, where the compositions and/or methods described herein are used for treatment of cancer, some specific therapeutic responses may include, but are not limited to, one or more of destroying neoplastic or cancerous cells, inducing apoptosis in neoplastic or cancerous cells, reducing the proliferation of neoplastic or cancerous cells, reducing metastasis of neoplastic cells found in cancers, tumor regression (i.e. shrinking the size (e.g., diameter or volume) of a tumor), enhanced immune memory against an existing cancer, and enhanced therapeutic efficacy of anti-cancer agents (e.g., immune checkpoint inhibitors), such as those described elsewhere herein (including those described with respect to combination therapies). Enhanced therapeutic efficacy of other treatments for cancer or comorbidities associated therewith may allow for decreased dosages of those treatments.

### Combination Therapies

Treatment of subjects with a microbubble composition as described herein may be combined with other treatments for the same disease that the payload is being administered to treat, such as cancer, and/or comorbidities associated therewith. Anti-cancer therapies, such as treatment with an anticancer agent and radiation therapy are well known in the art. These therapies can be administered to a subject according to any effective protocol, though the treatments may be modified as needed to optimize the combination treatment along with the microbubble composition. For example, and without limitation, radiation therapy is performed by administering to the subject a suitable radiation dose of a suitable time at any suitable interval according to well-established protocols. Anticancer agents are administered according to typical protocols for the given drug. Non-limiting classes of drugs that may be useful in combination with the microbubble compositions described herein include: tyrosine kinase inhibitors, such as gefitinib (Iressa™) and imatinib mesylate (Gleevec™); monoclonal antibodies, such as rituximab (Rituxan™) and cetuximab (Erbitux™); angiogenesis inhibitors, such as endostatin; immune modulators, such as interleukin-12 (IL-12) and interleukin-2 (IL-2); non-receptor tyrosine kinase inhibitors, such AG490 JAK2 inhibitor and PP2 src family kinase inhibitor or dasatinib; serine/threonine kinase inhibitors, such as U0126 for MEK1/2, wortmanin for PI3K; farnesyl or geranyl transferase inhibitors, such as FTI-277 and GGTI-298; G-protein-coupled receptor inhibitors, such as RC3095 for bombesin and An-238 for somatostatin; and indoleamine 2,3-dioxygenase (IDO) inhibitors, such as indoximod and epacadostat.

Non-limiting examples of specific anticancer agents include: AG-490; aldesleukin; alemtuzumab; alitretinoin; allopurinol; altretamine; amifostine; An-238; anastrozole; arsenic trioxide; asparaginase; BCG Live (Bacillus Calmette-Guerin); bevazizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; dasatinib; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-2777; fulvestrant; gefitinib; gemcitabine; gemcitabine; gemtuzumab ozogamicin; GGTI-298; goserelin acetate; gossypol; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin; and zoledronate.

Useful approaches to activating the adaptive immune response system (e.g., activating therapeutic antitumor immunity) include the blockade of immune checkpoints. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. Cytotoxic T-lymphocyte-associated antigen 4 (CTLA-4) antibodies were the first of this class of immunotherapeutics to receive FDA approval (ipilimumab). Inhibitors of additional immune-checkpoint proteins, such as programmed cell death protein 1 (PD-1) and programmed death-ligand 1 (PD-L1), have since been approved and demonstrated broad and diverse opportunities to enhance antitumor immunity with the potential to produce durable clinical responses.

PD-1, functioning as an immune checkpoint, plays an important role in down-regulating the immune system by preventing the activation of T cells, which in turn reduces autoimmunity and promotes self-tolerance. The inhibitory effect of PD-1 is accomplished through a dual mechanism of promoting apoptosis (programmed cell death) in antigen-specific T cells in lymph nodes while simultaneously reducing apoptosis in regulatory T cells (suppressor T cells). Therapeutics that block PD-1 binding, the PD-1 inhibitors (e.g., anti-PD-1 antibodies, anti-PD-L1 antibodies), activate the immune system to attack tumors and are therefore used to treat some types of cancer. Approved checkpoint inhibitors for PD-1 and PD-L1 include nivolumab, pembrolizumab, atezolizumab, avelumab, durvalumab, and cemiplimab. Additional inhibitory checkpoint molecules which may be targeted to provide immune checkpoint therapy (checkpoint blockade) include, but are not necessarily limited to, CD47, A2AR, B7-H3, B7-H4, BTLA, IDO, KIR, LAG3, NOX2, TIM-3, VISTA, and SIGLEC7.

According to certain aspects of the disclosure, the methods and/or compositions described herein may be administered in combination with immune checkpoint therapy, such as an immune checkpoint inhibitor (e.g., antibody), including any of the inhibitors described herein or any suitable inhibitor that targets an immune checkpoint molecule described herein. The methods and/or compositions herein, such as those that activate STING (e.g., delivery of CDNs such as cGAMP) may increase therapeutic efficacy of immune checkpoint therapy, as demonstrated in the examples.

### EXAMPLES

### Example 1. Spe-Dex MB Fabrication

Spe-Dex MBs were prepared as described in PCT/US2021/054820 to Lux et al., filed Oct. 13, 2021 and herein incorporated by reference in its entirety. Briefly, Potassium periodate (6.25 mmol) was added to a solution of Dextran 40 k (6.25 mmol of glucose monomers) in milliQ water (20 ml). The reaction was vigorously stirred in the dark for 7 h at room temperature and then spin filtered 2× using spin filters (MWCO 10 kDa, AMICON®) at 4,000 g for 10 min at 4° C. with water washing. The resulting retentate was dialyzed for 24 h against water using a regenerated cellulose semi permeable membrane (MWCO 3.5-5 kDa) and then added to a solution of spermine (2.96 mmol) in borate buffer (19 ml, 0.1 M, pH 11) over 5 h via syringe pump. The resulting solution was gently stirred for 24 h at room temperature followed by the addition of NaBH₄ (9.48 mmol) under ice bath and stirring for 48 h at room temperature. An additional portion of NaBH₄ (9.48 mmol) was then added and stirring continued for 24 h under the same conditions. Crude product was dialyzed against water (MWCO 3.5-5 kDa) for 48 h followed by lyophilization for 48 h to yield spermine-modified dextran (SpeDex).

For thiolation, SpeDex (3.55 μmol NH₂) was dissolved with phosphate-buffered saline (PBS) 1×, 5 mmol/l ethylenediaminetetraacetic acid (EDTA, 0.5 ml). To this a 1 mg/ml aqueous solution of 2-iminothiolane HCl (35.45 μmol) was added dropwise with vigorous stirring. The resulting mixture was stirred for 1 h, dialyzed for 48 h (MWCO 3.5 kDa), and lyophilized for 48 h.

SpeDex conjugation onto MBs was verified using fluorescently labeled SpeDex. Fluorescent labeling of SpeDex polymers was done using amine reactive 5/6-carboxyfluorescein succinimidyl ester (NHS-fluorescein). Briefly, SpeDex polymer (5 mg) was dissolved in 1 ml of 1× borate buffer (50 mmol/l, pH 8.5). NHS-fluorescein (5 mg, 10.562 μmol) was dissolved in DMF (0.5 ml) and added dropwise to SpeDex solution with vigorous stirring. The reaction was stirred for 1 h, dialyzed for 48 h (MWCO 3.5 kDa), and lyophilized for 48 h.

Microbubbles (MBs) were likewise formulated as previously reported. Briefly, lipid films containing a mixture of 1,2-distearoyl-sn-glycero-3-phosphocholine (DSPC), 1,2-Distearoyl-sn-Glycero-3-Phosphoethanolamine-N-[Methoxy(Polyethylene glycol)-2000] (DSPE-PEG2k), and 1,2-distearoyl-sn-glycero-3-phosphoethanolamine-N[maleimide(polyethylene glycol)5000](DSPE-PEG(5000)-mal) with a 90:5:5 molar ratio were prepared. These films were prepared by dissolving DSPC, DSPE-PEG(2000), and DSPE-PEG(5000)-mal in 100 μl of chloroform and slowly evaporating the mixture with a rotary evaporator (BUCHI ROTOVAPOR® R-100) until mostly dry. The resulting films were then dried overnight under vacuum and stored at −20° C. for later use. The lipid films were solvated in a mixture of PBS 1×/propylene glycol/glycerol (80:10:10 v/v/v, 2 ml total) and sonicated at 70° C. until clear or for 15 min. Perfluorobutane (PFB) vapor was then introduced in the solution, and the resulting mixture was tip sonicated at 70% amplitude for 5 seconds before being cooled down in an ice bath. The resulting MB formulation was washed with PBS 1× pH 6.5 plus 1 mmol 1-1 EDTA using centrifugation (300 g, 3 min) to yield PEGylated MBs with terminal maleimide functions (mal-MBs).

Thiolated SpeDex was then dissolved in the same PBS solution at 10 mg/ml and added to mal-MBs at a 1:20 maleimide:SpeDex molar ratio. The solution was rotated end-over-end for 4 h then washed (300 g, 3 min) and characterized using a Coulter Counter (BECKMAN COULTER® MULTISIZER™ 4) (data not shown). SpeDex conjugation onto MBs was confirmed via fluorescence microscopy and conjugation efficiency was quantitatively determined via flow cytometry to be approximately 100% of MBs being conjugated to SpeDex (data not shown).

### Example 2. Anti-CD11b Thiolation and Conjugation onto Spe-Dex MBs

To allow MBs to target APCs such as macrophages and dendritic cells (DCs), anti-CD11b antibodies (aCD11b) or isotype IgG (non-targeting control antibody) were thiolated and conjugated onto the surface of the MBs' maleimide groups. Anti-CD11b was first thiolated to allow for conjugation onto maleimide-bearing Spe-Dex MBs. Briefly, a 2 mg/mL solution of Traut's reagent (2-Iminothiolane) in PBS pH 8.0 with 5 mM EDTA was added at a 600:1 molar ratio to a solution of anti-CD11b in PBS with 50 mM EDTA. The resulting solution was rotated for 2 hours before having its buffer exchanged with PBS pH 6.5 with 1 mM EDTA using a ZEBA™ Spin desalting column (MWCO 7 kDA) (THERMO SCIENTIFIC™). The extent of thiolation was determined using the Measure-IT™ assay (THERMO SCIENTIFIC™) and was reported to be 1.67 thiols per antibody. Spe-Dex MBs were then added to the solution of anti-CD11b at a ratio of 0.345 equivalents of antibody per maleimide and rotated end-over-end for 15 hours at 4° C. to allow for conjugation. Afterwards, the solution was washed 3× with PBS at 300 g for 3 min.

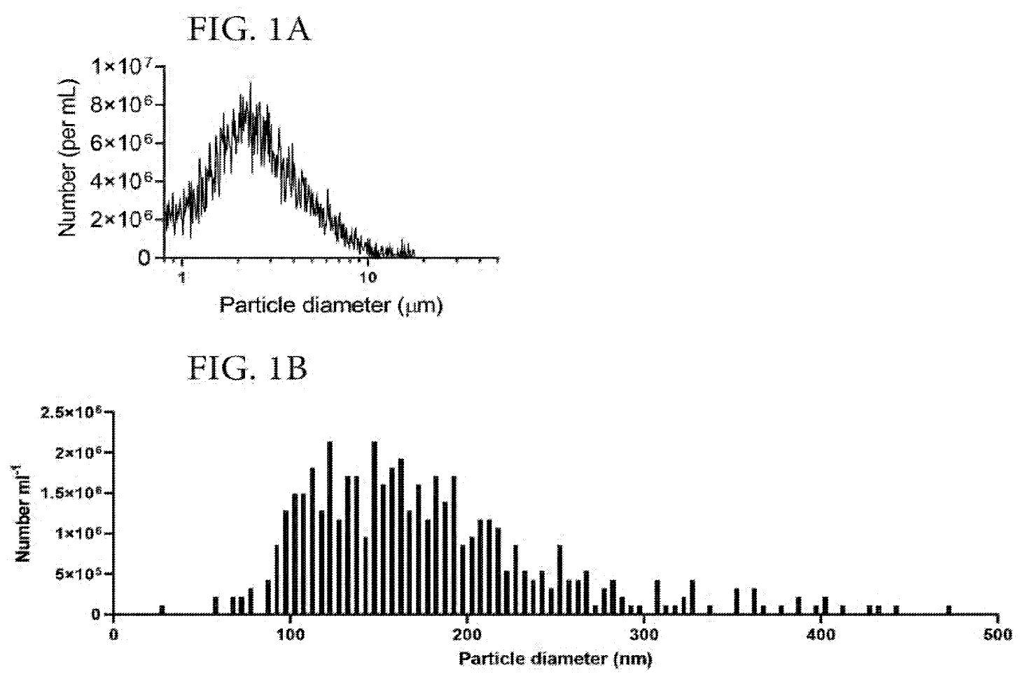

Flow cytometry and fluorescence microscopy were used to confirm conjugation of APC-conjugated aCD11b onto SpeDex MBs. The thiol-maleimide coupling reactions were highly efficient, with flow cytometry showing more than 99% of MBs conjugated with both SpeDex and aCD11b on their surface (data not shown). The conjugation resulted in SpeDex- and aCD11b-conjugated MBs (cMBs) having a size distribution of 1-10 m with a mean size of 2.6 m, as measured by Coulter Counter (FIG. 1A). The number of aCD11b on SpeDex MBs was measured by running known quantities of antibody on a SDS PAGE gel and measuring the intensities of their bands. Using this standard curve resulted in approximately 291,774 aCD11b molecules per cMB (data not shown).

To validate targeting specificity, both Spe-Dex MBs and cMBs were incubated with cGAMP at a nitrogen:phosphate (N:P) ratio of 1:34, fluorescently labeled with 1,1′-dioctadecyl-3,3,3′,3′-tetramethylindodicarbocyanine (DiD), and then added to either 300,000 THP-1 macrophages (CD11b+) or EO771 murine breast cancer cells (CD11b−) plated on 12-well plates. The wells were washed three times, filled with Perfluorobutane (PFB)-saturated PBS, and imaged with brightfield and fluorescence microscopy. The Spe-Dex MBs exhibited no targeting to the cells. Both bright field and fluorescence microscopy revealed an abundance of cell-ncMB complexes after incubation of ncMBs with THP-1 macrophages but not with EO771 cells (data not shown). These results show that ncMBs have specific targeting to APCs.

### Example 3. Formation of SpeDex-cGAMP Nanocomplexes and Loading of MBs

Two strategies were used to load cGAMP and form ncMBs. The first involved adding cGAMP to cMBs, as described in Example 2. This approach was used throughout the remaining examples, unless specified otherwise. A fluorescent analog of cGAMP (FITC FITC-cGAMP) was used to verify loading onto SpeDex SpeDex-aCD11b MBs via fluorescent microscopy, flow cytometry, and spectrophotometry. Flow cytometry indicated that this approach resulted in highly efficient binding with more than 98% of cMBs carrying cGAMP. As shown in Table 1 below, analyzing the infranatant of the loaded cMBs with a spectrophotometer after washing indicated an average load of 1.25×10⁸ cGAMP molecules per ncMB. MBs without SpeDex did not have any measurable loading of cGAMP, suggesting that SpeDex is a necessary component for cGAMP loading. These results show that ncMBs have high conjugation efficiency and high cGAMP loading.

TABLE 1
Loading of cGAMP onto cMBs

| | | | | | | MBs | |
|---|---|---|---|---|---|---|---|
| | | | | | # | Surface | |
| | % | μg | # | # | cGAMP/ | Area | pg/ |
| Sample | loading | cGAMP | cGAMP | MBs | MB | (μm²) | μm² |
| With | 41.44 | 4.59 | 2.50E15 | 2.00E7 | 1.25E8 | 3.31E8 | 0.0139 |
| SpeDex | | | | | | | |
| Without | 0.00 | −1.28E−15 | −0.70 | 2.70E7 | −2.58E-8 | 3.31E8 | 0.0000 |
| SpeDex | | | | | | | |

Nanocomplex decorated microbubbles (ncMBs) can also be formulated by first preparing cGAMP SpeDex nanocomplexes and conjugating them to the MB surface in a one pot reaction with aCD11b. Briefly, a solution of SpeDex polymer was added to a solution of cGAMP at equal volumes and at a N:P ratio of 1:10. The mixture was vortexed and then incubated for 30 min. The size distribution of the resulting cGAMP-SpeDex nanocomplexes was evaluated using nanoparticle tracking analysis (NTA) (PARTICLE METRIX™, ZETAVIEW®) and is shown in FIG. 1B. The average nanocomplex size was determined to be 168.8±9.4 nm. Nanocomplexes were then conjugated onto maleimide-bearing MBs at a 1:20 maleimide:SpeDex molar ratio and rotated for 4 h followed by three washes with PBS to afford nanocomplex-conjugated MBs (ncMBs). ncMBs were characterized using a Coulter Counter (BECKMAN COULTER® MULTISIZER™ 4) (data not shown). Flow cytometry data of MBs conjugated with FITC labeled SpeDex and DY547-c-diGMP nanocomplexes showed that approximately 98% of MBs were conjugated with SpeDex polymer and approximately 80% of MBs carried the DY547-c-diGMP payload (data not shown).

### Example 4. Sonoporation of Mouse Bone Marrow-Derived Macrophages and THP-1 Macrophages Using diGMP Loaded Spe-Dex-CD11b MBs

To show that ncMBs can efficiently deliver CDNs to the cytosol of APCs, ncMBs loaded with a fluorescently labeled CDN (DY547-c-diGMP) were incubated with macrophages and then sonoporated.

Six days before treatment, human THP-1 monocytes were spun down and resuspended in RPMI, 20% FBS, 1% penicillin/streptomycin (P/S). Phorbol 12-myristate 13-acetate (PMA) was added to obtain a final concentration of 200 nM to differentiate the monocytes into macrophages. 1 mL per well of the cell solution was plated in a 12 well plate (400,000 cells/well) and left to differentiate for 3 days. Afterwards, cells were washed once with PBS then replenished with fresh RPMI.

Mouse bone marrow-derived macrophages (BMDMs) were isolated from the hind leg femur bone marrow of C57BL/6J mice and were cultured or activated with macrophage colony-stimulating factor (M-CSF, 50 ng ml-1) according to standardized procedures. The purity of the induced cells was assessed by flow cytometry for CD45+CD11b+ cells.

One day before treatment, cells were plated at 400,000 cells/well in a 12 well plate. On the day of treatment, Spe-Dex-CD11b MBs (5 MBs/cell) were rotated with 10 nmol cGAMP for 15 min to allow for binding to MBs, yielding ncMBs. Cells were washed once, media was aspirated off, and the entire suspension of cGAMP-loaded MBs was added to the wells. Plates were flipped upside down and left to incubate for 10 min at 37° C. Cells were diluted to 2.5 mL with PFB-saturated RPMI, placed on top of a water bath set to 37° C., then sonoporated at 1 MHz, with 1 W/cm² power density, 20% duty cycle (DC) for 60 seconds (SONITRON® GTS, 15 mm diameter). Fluorescence microscopy images were taken at different time points (0, 3, 6, 12, 24, and 48 h).

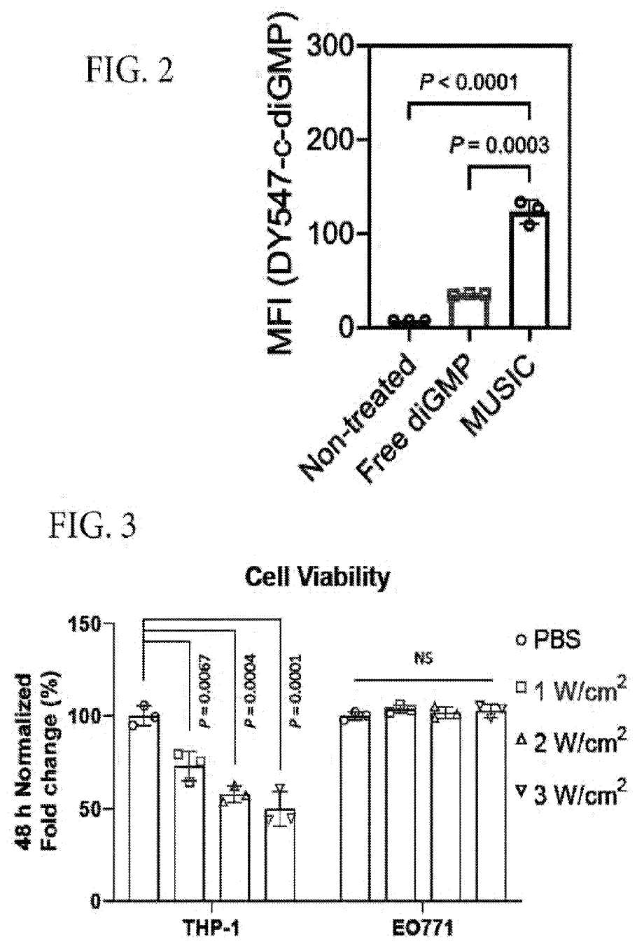

Sonoporation of BMDMs using DY547-diGMP loaded Spe-Dex MBs showed high uptake of diGMP in virtually all of the cells, whereas the diGMP only sample showed weak uptake and high background signal. Mean fluorescence intensity (MFI) of the BMDMs revealed that MUSIC treatment increased the uptake of CDNs by more than two folds relative to uptake of free CDN, as shown in FIG. 2. THP-1 macrophages showed no STING activation, possibly because the fluorescent diGMP is too large to fit into the active pocket of STING.

To assess sonoporation toxicity, cell viabilities of EO771 and THP-1 cells were measured 48 h after MUSIC treatment at 1 W/cm². Briefly, Propidium Iodide READY FLOW™ Reagent (THERMOFISHER®) was added to MUSIC-treated cells after 48 hours of incubation. Flow cytometry assays were performed for distinguishing dead cells from live cells. Fluorescence associated with cell viability was measured, which showed no loss of viability in EO771 cells and approximately a 25% viability decrease in THP-1 cells, as shown in FIG. 3. These results show that ncMBs have improved cytosolic delivery of CDNs into macrophages and acceptable toxicity.

### Example 5. Treatment of Macrophages Using cGAMP Loaded Spe-Dex-CD11b MBs (MUSIC)

Mouse BMDMs and human THP-1 cells were cultured as described above. Primary human peripheral blood monocyte derived macrophages underwent induced differentiation using CD11b+ cells sorted from PBMC (STEMCELL TECHNOLOGIES®) by culturing cells with macrophage colony-stimulating factor (M-CSF, 50 ng/ml) for 7 days.

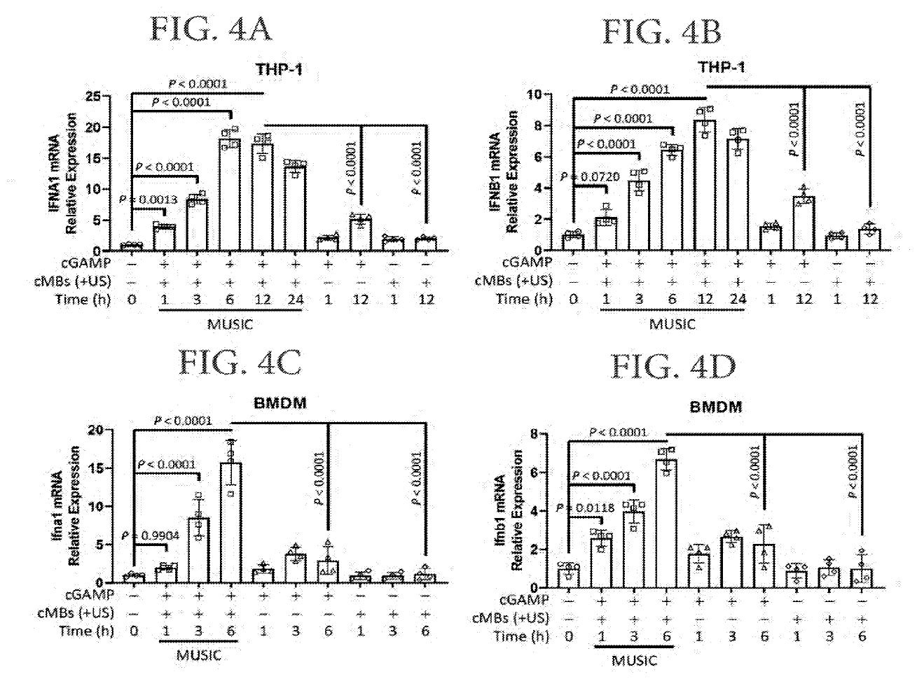

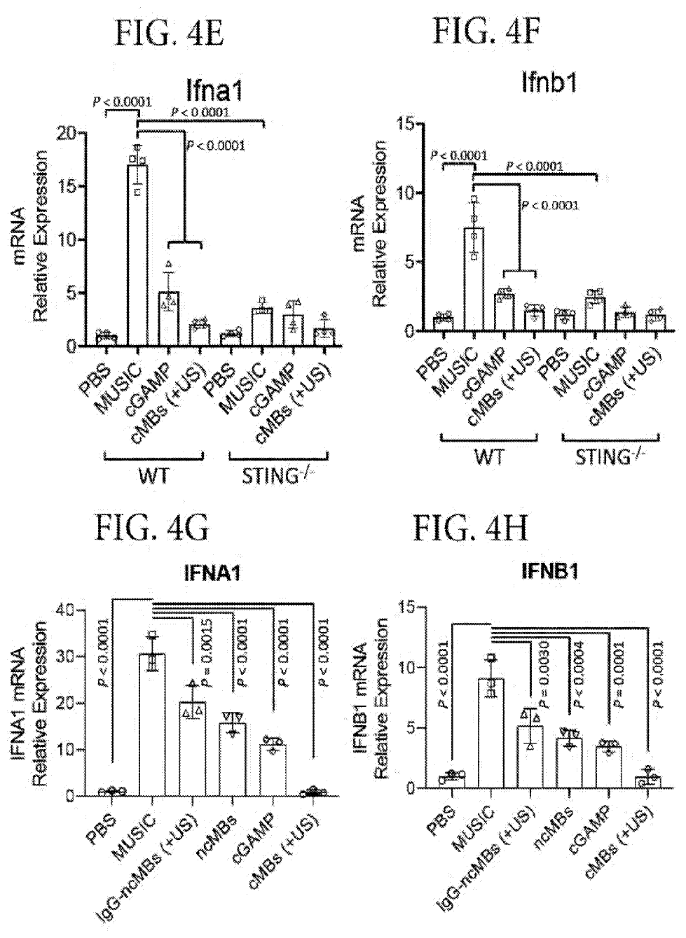

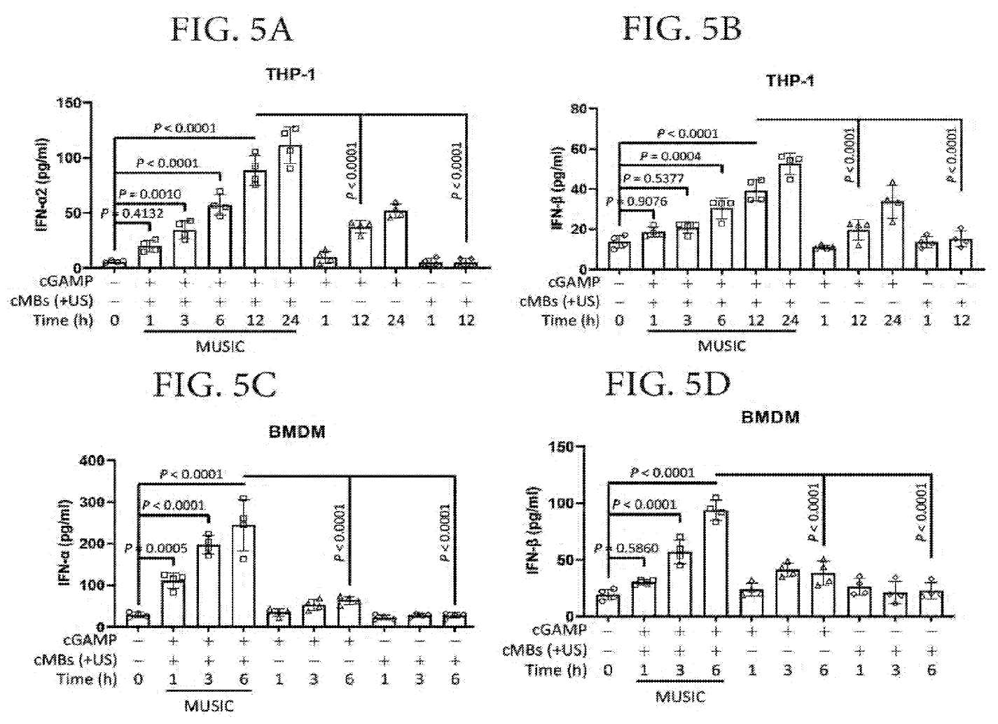

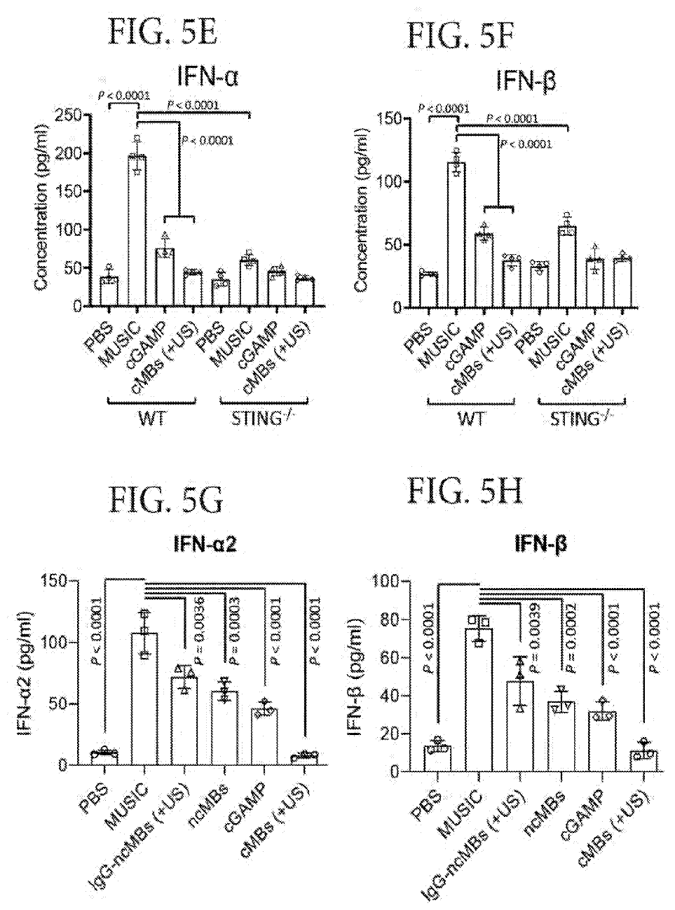

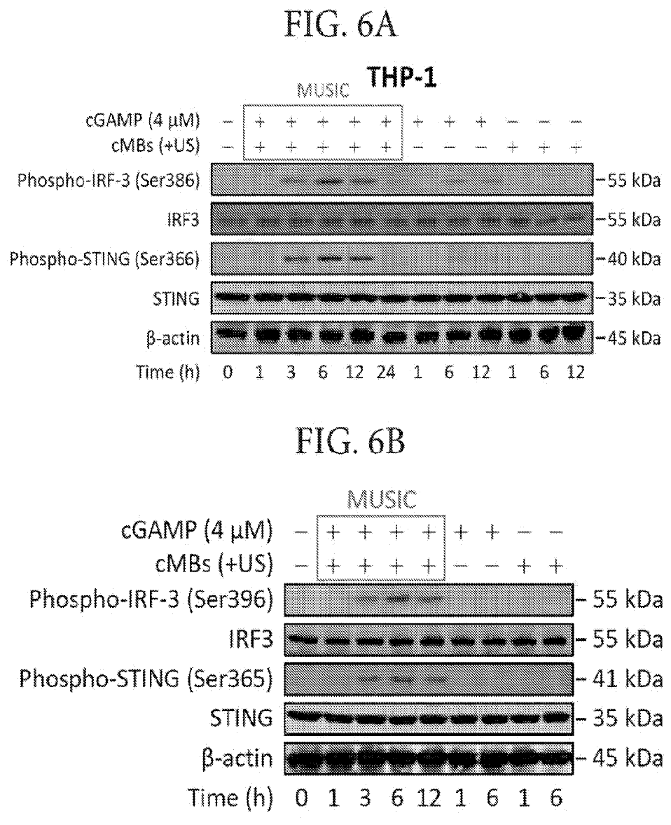

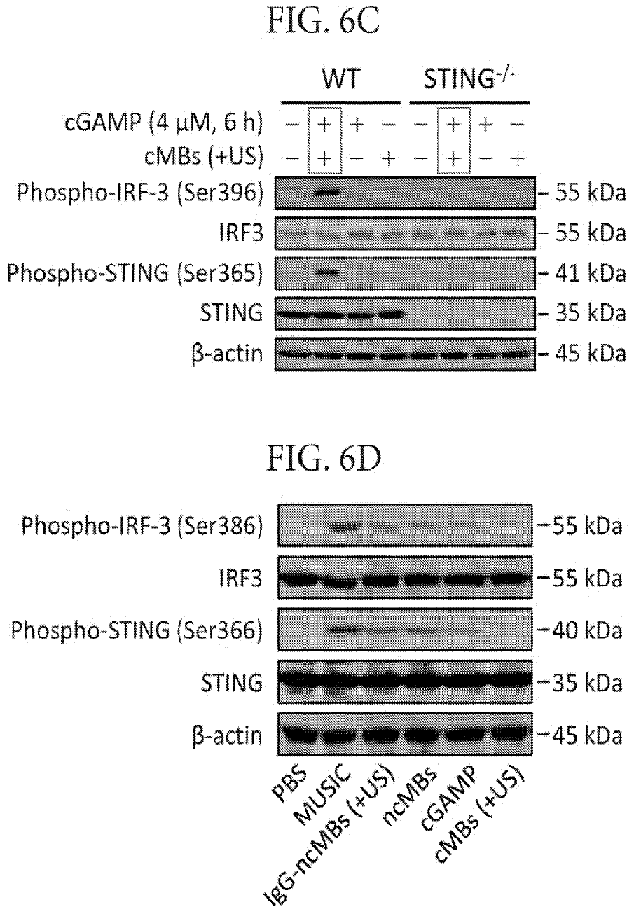

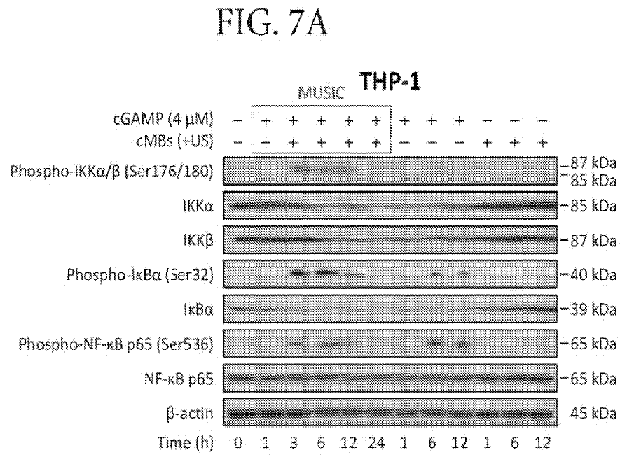

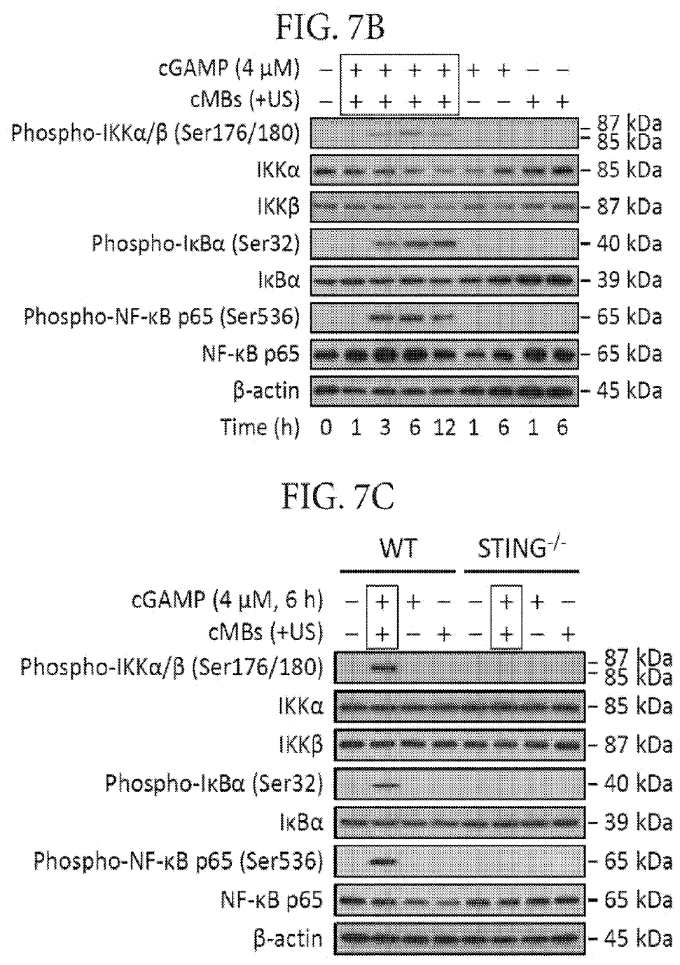

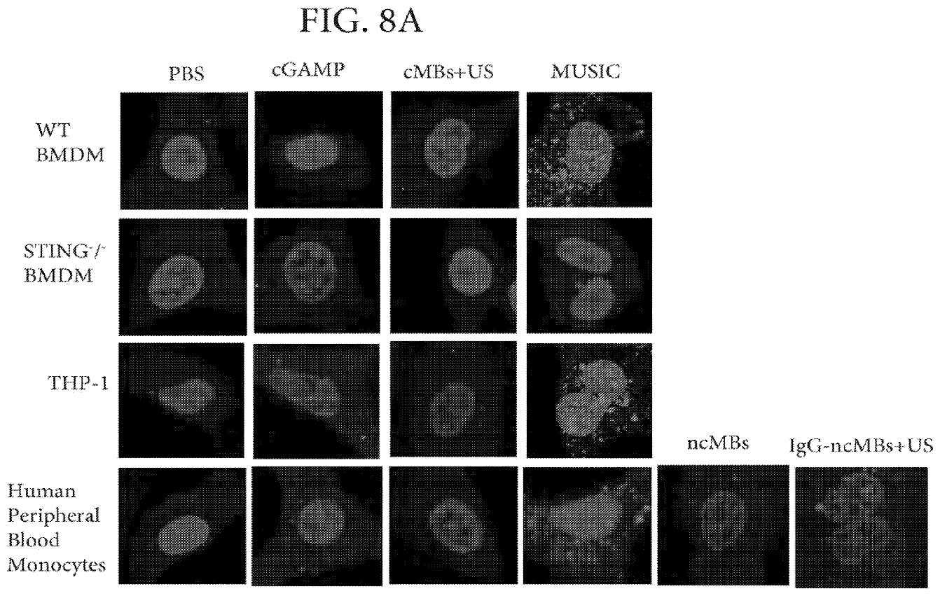

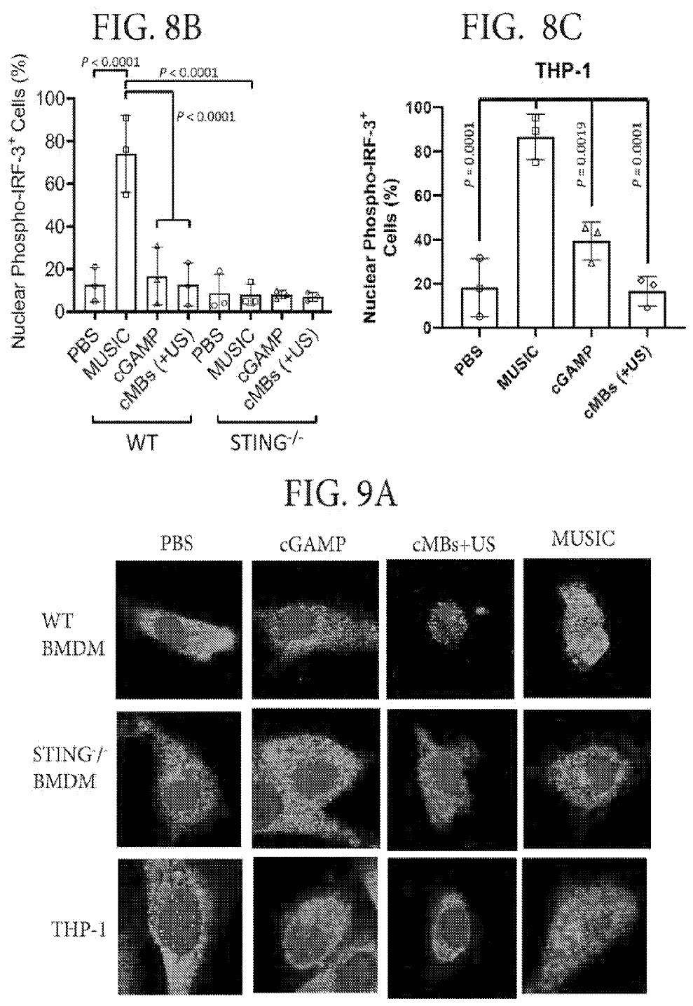

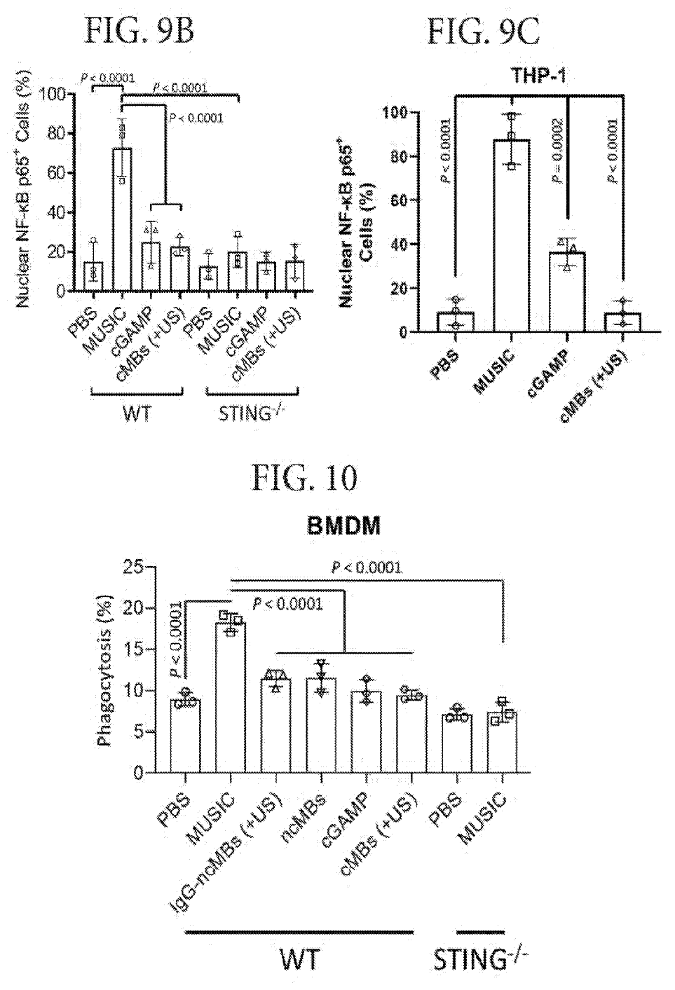

All macrophages were plated and treated as described in Example 4. cGAMP only (diluted in sterile PBS), cMBs+ultrasound (US) only, PBS, STING knockout cells (BMDM), and IgG-ncMBs+US (peripheral blood monocyte derived macrophages) were used as controls. Cells were then collected at various time points post-treatment for qRT-PCR (FIGS. 4A-4H), ELISA (FIGS. 5A-5H), western blot (FIGS. 6A-6D; FIGS. 7A-7D), and immunostaining experiments (FIGS. 8A-8C; FIGS. 9A-9C).

Although no STING or IRF-3 phosphorylation was observed in BMDMs treated with cGAMP alone, a small degree of activation of the immune sensors was noted in THP-1 cells (FIG. 6A), likely due to limited uptake of cGAMP by previously identified transporters on the plasma membrane of human cells. To confirm that IRF3 activation and downstream inflammatory responses are mediated by STING, BMDM from STING−/− mice were treated with MUSIC. As shown in FIG. 6C, no type I IFN responses were observed in STING−/− mice, therefore confirming that the immune response generated by MUSIC and the activation of downstream effectors are STING-dependent.

To demonstrate that the activation of STING leads to the mobilization of downstream signal cascade and transcriptional activities, the nuclear translocation of phosphorylated IRF3 (pIRF3), which acts as a transcription factor for proinflammatory genes, was examined at 6 h after treatment, as shown in FIG. 8A. Quantification of nuclear fluorescent positive cells was done by randomly measuring 500 cells in each 60 group (n=3), as shown in FIG. 8B for BMDMs and FIG. 8C for THP-1 cells. While some nuclear translocation of pIRF3 was observed only in in THP-1 cells treated with cGAMP alone, significantly higher nuclear translocation was observed in MUSIC-treated THP-1 cells compared to all controls.

NF-κB, another major downstream component of the STING pathway, was also activated upon MUSIC treatment, as shown by the phosphorylation of IKKα/β, IκBα, and p65 in THP-1 cells and mouse BMDMs, but was not observed in BMDMs from STING^{-/-} mice, as shown in FIGS. 7A-7C. The activation of NF-κB signaling is supported by the increased translocation of p65 into the nucleus in both BMDMs and THP-1 cells after MUSIC treatment, as shown in FIG. 9A, where it acts as a transcription factor for proinflammatory cytokine genes. Quantification of nuclear fluorescent positive cells was done by randomly measuring 500 cells in each 60 group (n=3), as shown in FIG. 9B for BMDMs and FIG. 9C for THP-1 cells. Indeed, the expression of IFN genes was higher in MUSIC-treated THP-1 cells and mouse BMDMs compared to cells treated with cGAMP alone (FIGS. 4A-4D), which correlated with a greater production of the proteins (FIGS. 5A-5D). In contrast, APCs from STING^{-/-} mice showed no IFN mRNA expression (FIGS. 4E-4F) or protein expression (FIGS. 5E-5F), supporting that the effects of MUSIC treatment rely on STING signaling.

Treatment using MUSIC significantly increased the relative interferon mRNA expression in both THP-1 and BMDMs when compared to cGAMP alone at all time points, as shown by qRT-PCR results. The increased interferon mRNA expression correlated with a higher concentration of interferon proteins in the supernatant of the cells when compared to cGAMP alone as shown by ELISA. Western blot showed phosphorylation of STING and the downstream effectors IRF3 and NF-kB in MUSIC-treated samples, suggesting that the interferon production shown from qRT-PCR and ELISA is a direct result of STING activation. cGAMP alone caused slight phosphorylation of STING and IRF3 but not as much as with MUSIC. For NF-kB, the phosphorylation caused by cGAMP alone in THP-1 macrophages is comparable to that caused by MUSIC, probably because THP-1 macrophages have a cGAMP receptor that can cause NF-kB phosphorylation. BMDMs do not have this receptor, which is why cGAMP alone showed no phosphorylation of any of the proteins. After phosphorylation, IRF3 and NF-kB translocate to the nucleus, which was visualized using immunostaining. This translocation was not visible in cGAMP only samples. MB only samples showed no effect in any of the experiments when compared to negative controls. Furthermore, repeating the experiments using STING^{-/-} cells showed loss of efficacy. STING^{-/-} cells showed a complete reduction in mRNA expression, interferon expression, phosphorylation, and translocation, giving further proof that all these effects were due to STING pathway activation. Experiments in primary human peripheral blood monocyte derived macrophages produced similar results, thus confirming the specificity and efficacy of MUSIC in activating STING signaling in human primary APCs.

### Example 6. Phagocytosis of EO771 Breast Cancer Cells by MUSIC-Treated Mouse Bone Marrow-Derived Macrophages (BMDMs)

In addition to increased cytokine production, MUSIC was also able to enhance the phagocytosis ability of treated macrophage, which is consistent with previous findings that STING activation in macrophages can improve their phagocytosis functions. Briefly, BMDMs were cultured for 6 h after treatment as described in Example 5. EO771 cells were pre-stained with Far Red for 2 h, and then co-cultured with the BMDMs at a ratio of 1:1. Phagocytosis was measured by flow cytometry after 4 h co-culture. Quantification of phagocytized EO771 cells by BMDMs from three biologically independent experiments is shown in FIG. 10.

### Example 7. OT-I and II Cell Proliferation Using BMDMs and Tumor-Associated Macrophages (TAMs) Treated with MUSIC

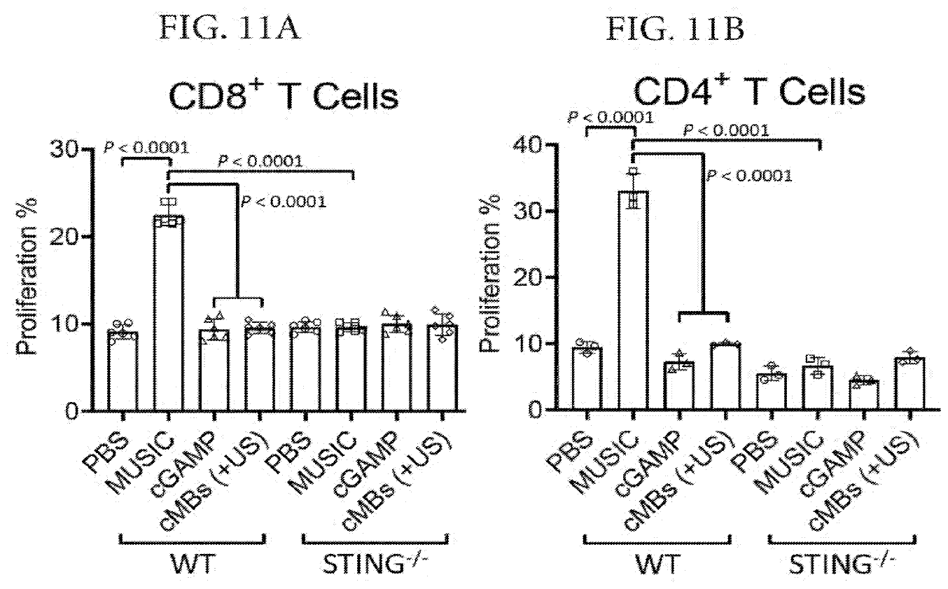

Macrophages are professional APCs that can prime T cells. This process can be amplified through activation of STING, as macrophages that have had their STING pathway activated are better able to present antigens to prime T-cells, causing their proliferation and resulting in potent antitumor immunity. This was confirmed using T-cells engineered to recognize OVA antigen (OT cells). BMDMs were treated with MUSIC as described in Example 5 and then incubated with either an MHC-I or MHC-II binding OVA peptide (amino acids 257-264 or 323-339, respectively) for 6 hours. Afterwards, macrophages were washed then incubated for 72 hours with either OT-I cells if they received the MIC-I binding peptide or OT-II cells if they received the MHC-II binding peptide. The proliferation of OT cells was then quantified using flow cytometry. As shown in FIG. 11A-11B, proliferation of both CD4⁺ and CD8⁺ T cells was increased when co-cultured with MUSIC-treated BMDMs relative to other treatment groups. This enhanced T-cell priming effect by MUSIC was absent in STING−/− BMDMs, suggesting that it is a STING-dependent response. OT-I cells (which are CD8*) had approximately a 2.5× increase in proliferation when the BMDMs were treated with MUSIC compared to negative controls. OT-II cells (which are CD4⁺) had approximately a 3.5× increase when compared to negative controls.

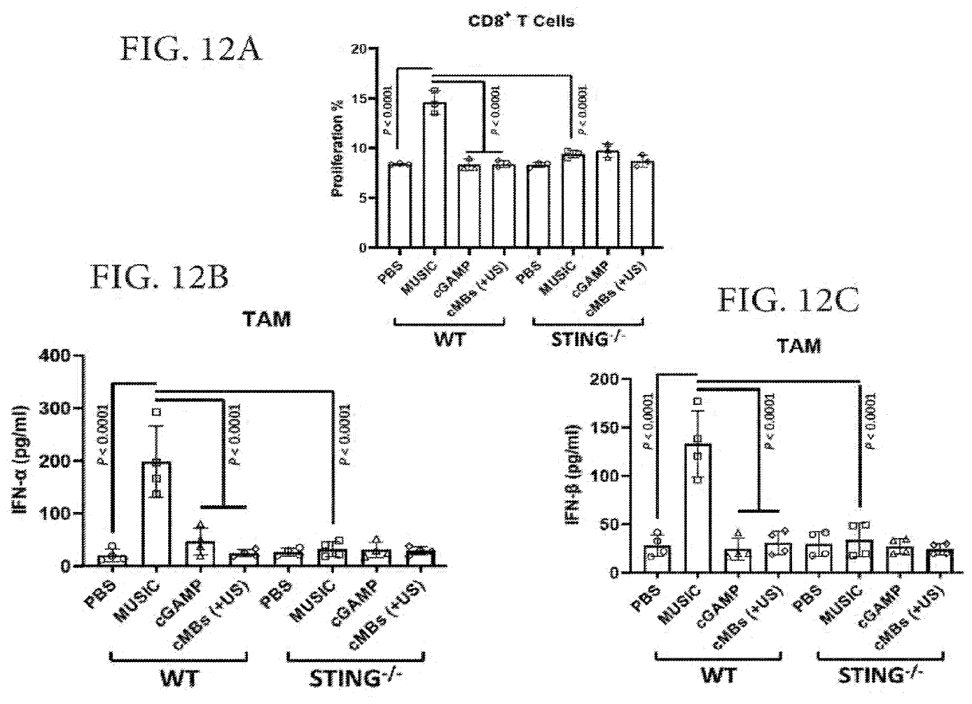

In addition to BMDMs, MUSIC treatment of tumor-associated macrophages (TAMs) from EO771 tumors implanted in wild-type mice also potentiated T-cell priming (FIG. 12A) and induced IFN protein expression (FIG. 12B-12C) but had no effect on TAMs from STING^{-/-} mice, thus supporting the potential of MUSIC to induce anti-tumor responses in vivo.

### Example 8. Treatment of Mouse Bone Marrow-Derived Dendritic Cells (BMDCs) Using MUSIC

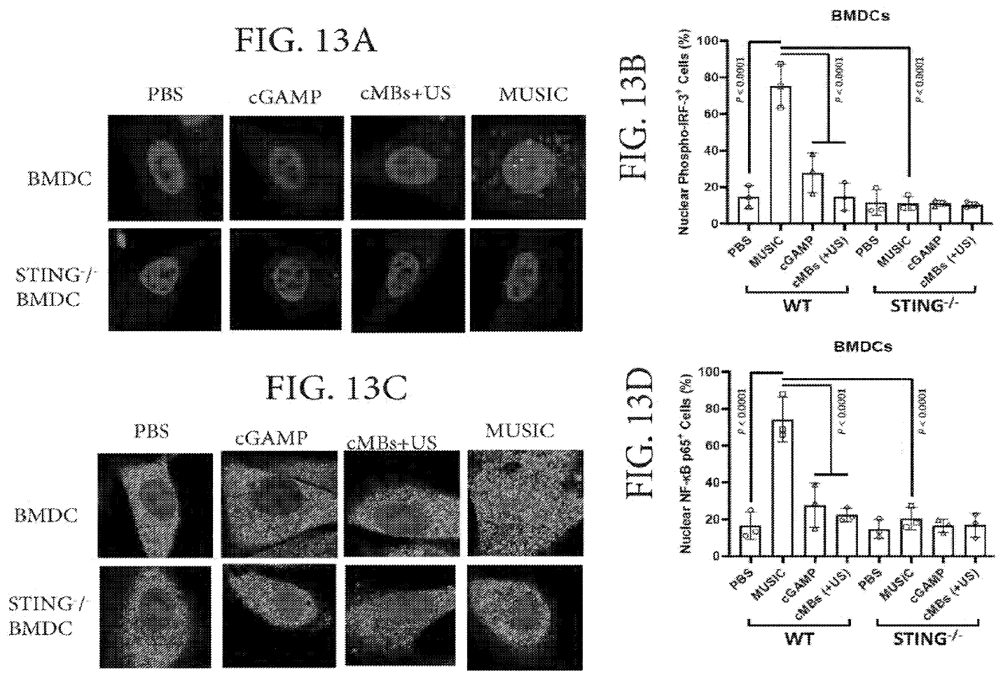

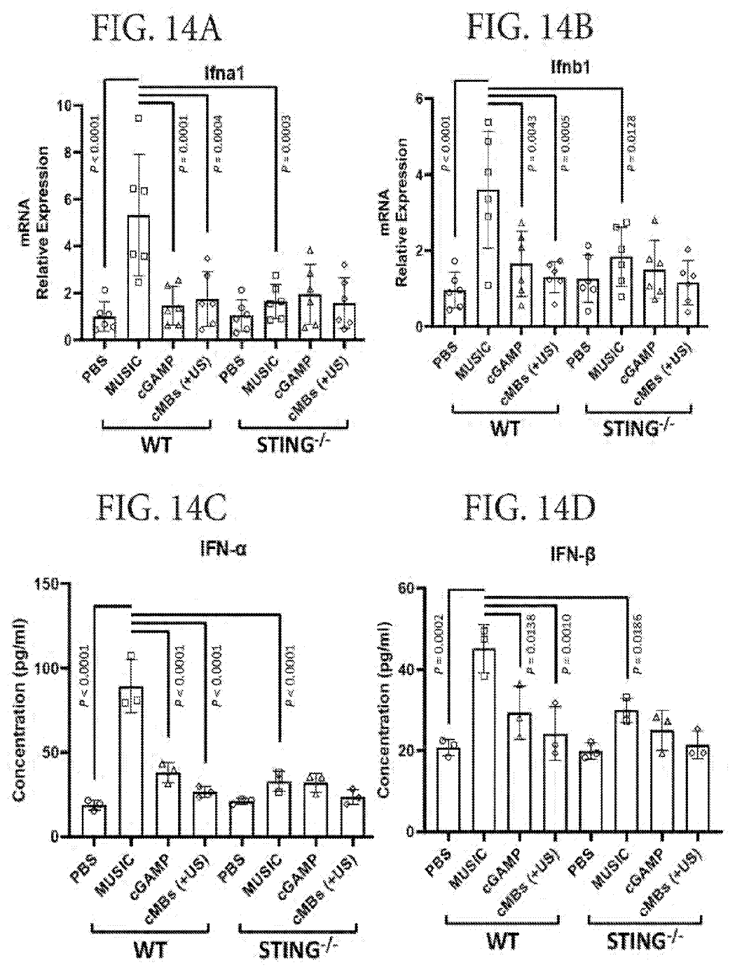

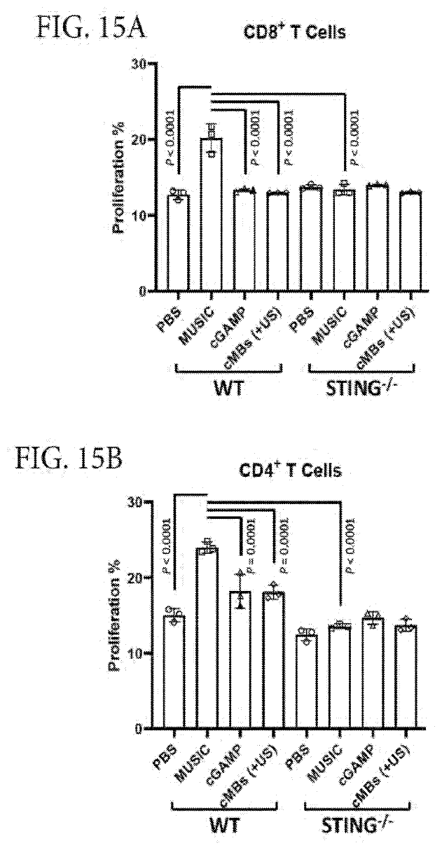

Since dendritic cells (DCs) are another major type of professional APC, bone marrow-derived dendritic cells (BMDCs) were also treated with MUSIC. Mouse BMDCs were isolated from the hind leg femur bone marrow of C57BL/6J mice and were cultured or activated with granulocyte macrophage colony-stimulating factor (GM-CSF, 20 ng/ml), according to standardized procedures. The purity of the induced cells was assessed by flow cytometry for CD45⁺CD11c⁺ cells. Similar robust STING and down-stream IRF3/NF-κB activation (FIGS. 13A-13D), increased type I IFN responses (FIGS. 14A-14D), and enhanced priming of antigen-specific T cells (FIGS. 15A-15B) were observed. Together, these findings demonstrated that MUSIC can effectively enhance STING activation in APCs, leading to improved priming of T cell responses.

### Example 9. In Vivo CDN Delivery

An orthotopic syngeneic murine breast cancer model was used to test the effectiveness of the MUSIC platform in activating the STING pathway in vivo in syngeneic hosts. Briefly, 1 million EO771 breast cancer cells in 50 μL PBS were injected subcutaneously into the lower mammary fat pad of 6-week old female C57BL/6J mice. The delivery efficiency of CDNs in vivo was first assessed using DY547-c-diGMP loaded ncMBs conjugated with either IgG (non-targeting) or aCD11b (targeting). The mice were injected intratumorally with 20 μL of solution infused at 1 μL/second using a syringe pump for a dose of 2.8×10⁷ MBs.

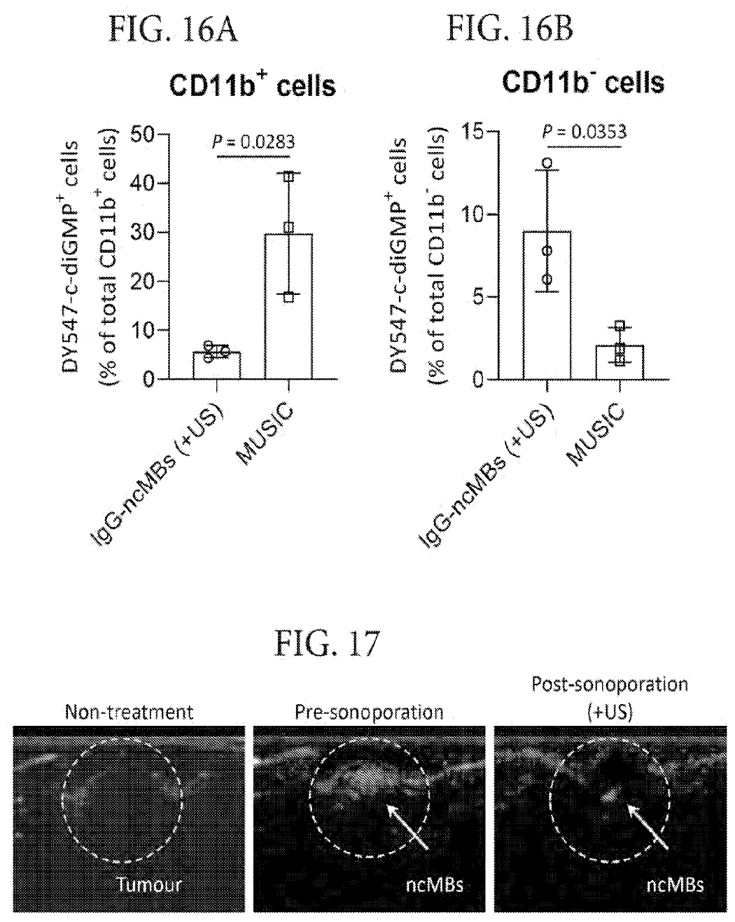

Two hours after ultrasound treatment (hereinafter ncMBs IgG or MUSIC) single cell suspensions from tumor tissues were isolated for flow cytometry analysis. CD11b^{+/-} cells with DY547-c-diGMP positive signals were measured and compared. Quantification of the cells as gated in each group (n=3) is shown in FIGS. 16A-16B, evaluated using an unpaired Student t-test. The delivery of DY547-c-diGMP in tumor-associated CD11b+ cells was more than 7-fold higher when using ncMBs as compared to non-targeted IgG-ncMBs. Importantly, non-specific uptake of CDNs in CD11b− cells was negligible and 4-fold lower with ncMBs as compared to the IgG-ncMBs.

### Example 10. In Vivo STING Activation

Orthotopic mammary fat pad tumors were established as in Example 9. The tumors were grown for 13 days before mice were randomized into treatment group and controls groups based on tumor size: PBS only, cGAMP only, ncMBs only, cMBs+US only, IgG-ncMBs+US, and MUSIC. The mice had an average tumor volume of 100 mm³ and any differences between the means were determined to not be statistically significant using ANOVA. The mice were injected intratumorally with 20 μL of solution infused at 1 μL/second using a syringe pump. A dose of 100 μg of cGAMP was used for all cGAMP groups and a dose of 2.7×10⁷ MBs was used for all MB groups. The mice were treated every other day for a total of three treatments (days 13, 15, and 17). US, where part of the treatment group, was applied by using acoustic coupling gel and a 1-MHz plane wave transducer operating at 4 W/cm² for 60 seconds and a 50% duty cycle given to opposite sides of the tumor for a total treatment time of 120 seconds. At 18 days post tumor inoculation treated mice were sacrificed.

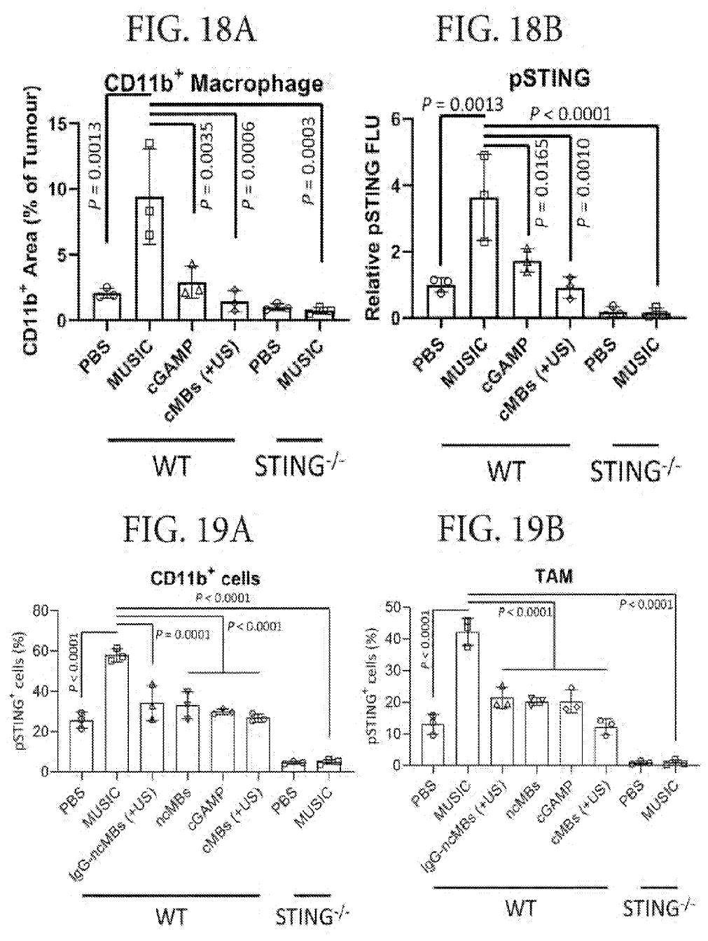

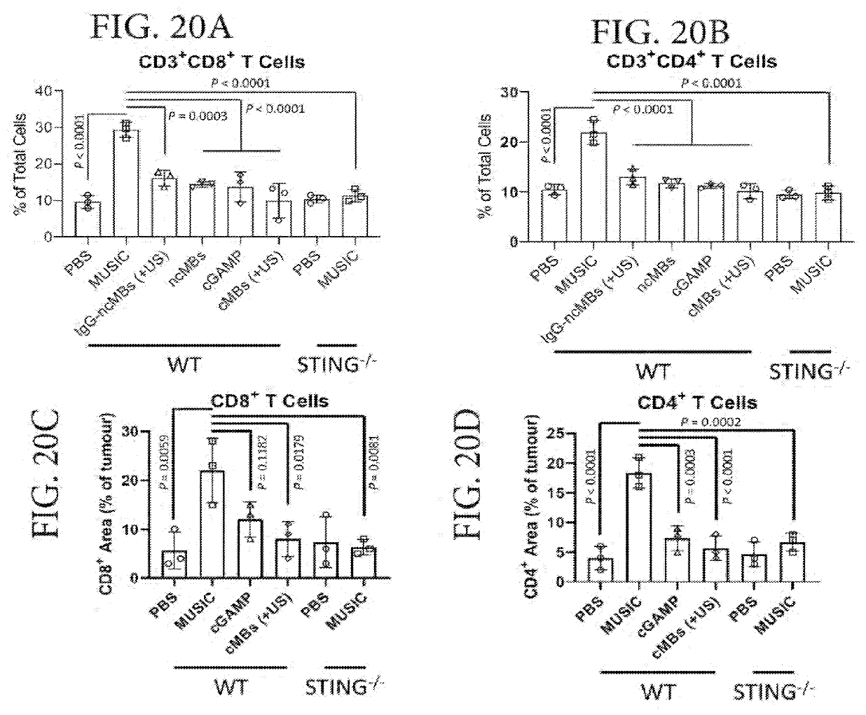

MUSIC enabled imaged-guided delivery of CDNs in vivo is shown in FIG. 17. Immunohistochemical staining of tumor sections three days after treatment (not shown) confirmed that MUSIC prevented Ki67 expression, indicating inhibited tumor cell proliferation, but had no direct effects on other tissues. Immunostaining by confocal microscopy was used to visualize recruited CD11b+ cells and pSTING+ cells in tumor paraffin section slides. Fluorescence intensity was measured and compared by IMAGEJ™ software from three randomly selected images, as shown in FIGS. 18A-18B. Single cell suspensions from tumor tissues were also collected for flow cytometry analysis, with CD11b, CD68, and IL-10 165 being used to gate TAMs. CD11b+ cells and TAMs with pSTING positive signals were measured and compared for each group (n=3), as shown in FIGS. 19A-19B. Evaluation of the tumor immune microenvironment revealed increased phosphorylation of STING in MUSIC-treated tumor tissues, most preferentially in CD11b+ cells. The increased phosphorylation of STING in CD11b+ cells correlated with increased recruitment of CD8+ and CD4+ T cells into the tumor after MUSIC treatment as quantified by flow cytometry, as shown in FIGS. 20A-20B. Activated CD8+ T cells and CD4+T cells were also detected and quantified by immunostaining in tumor paraffin section slides, as shown in FIGS. 20C-20D.

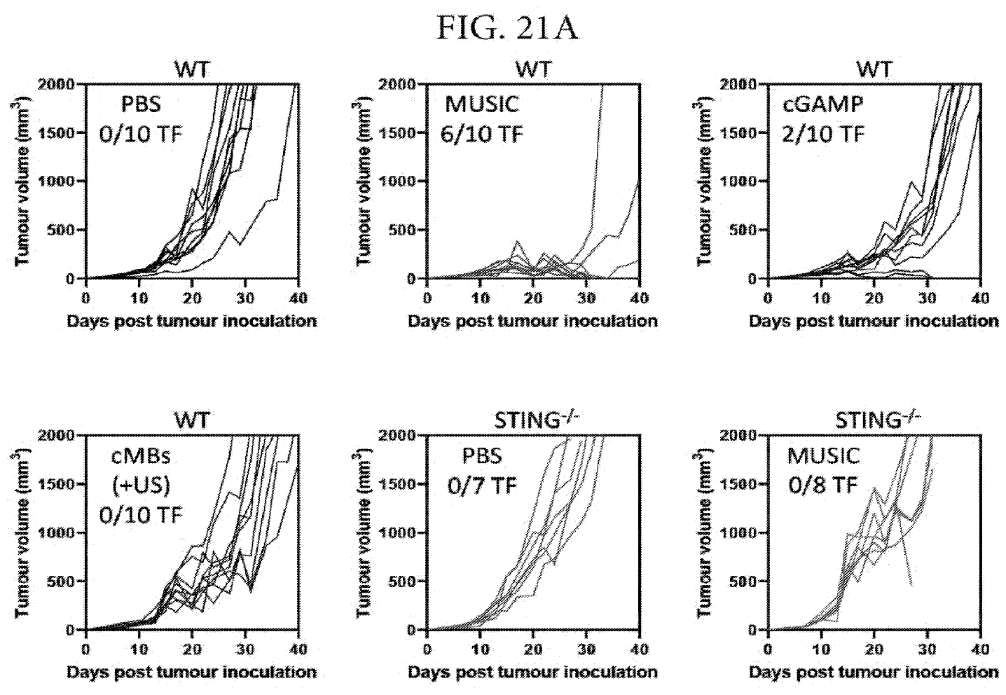

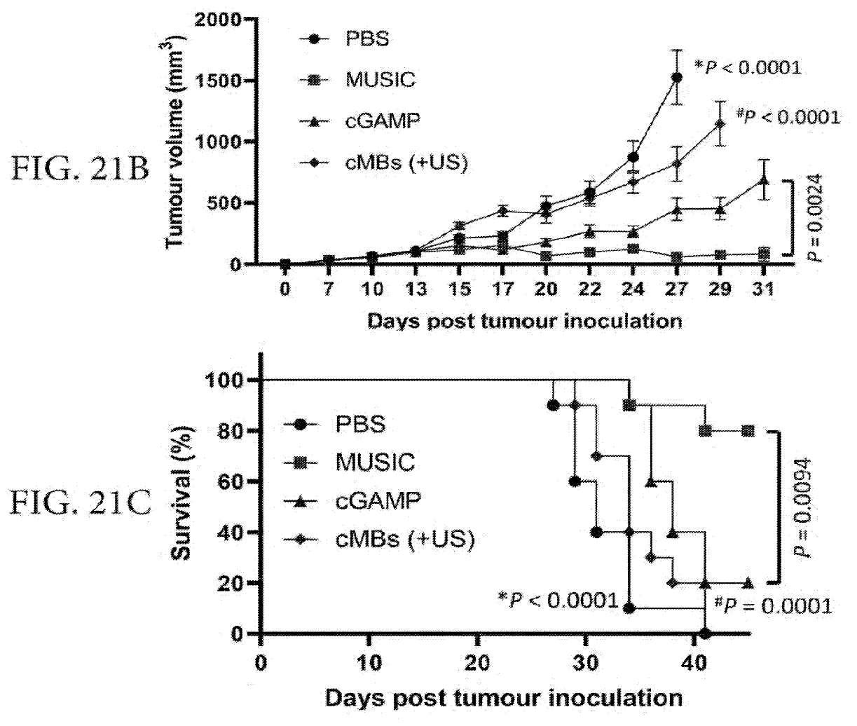

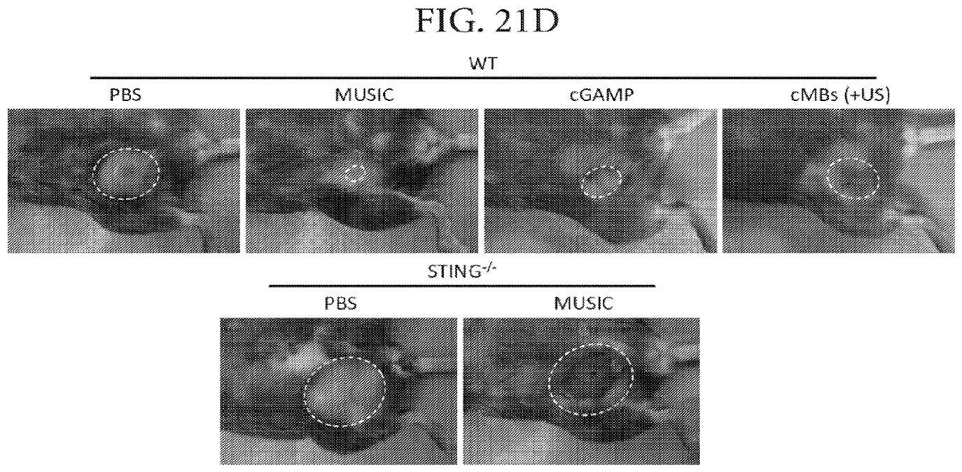

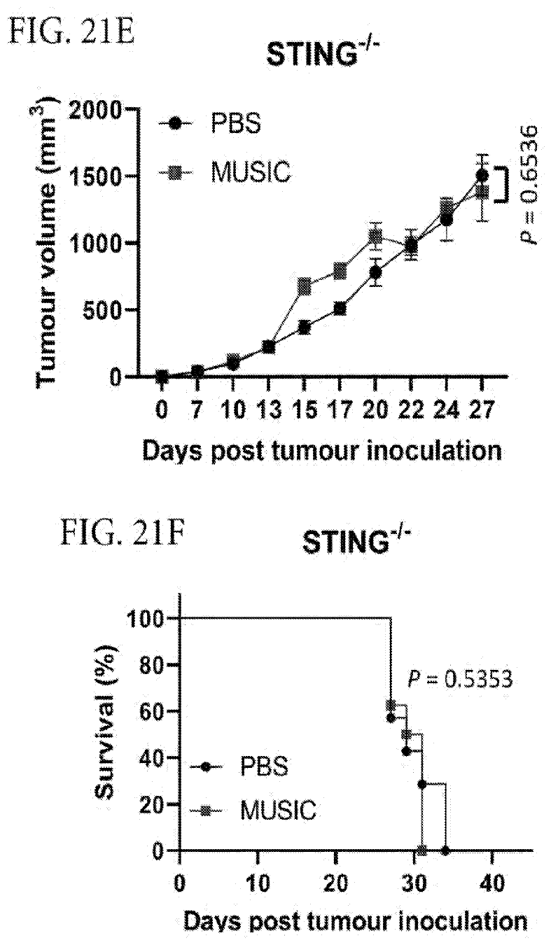

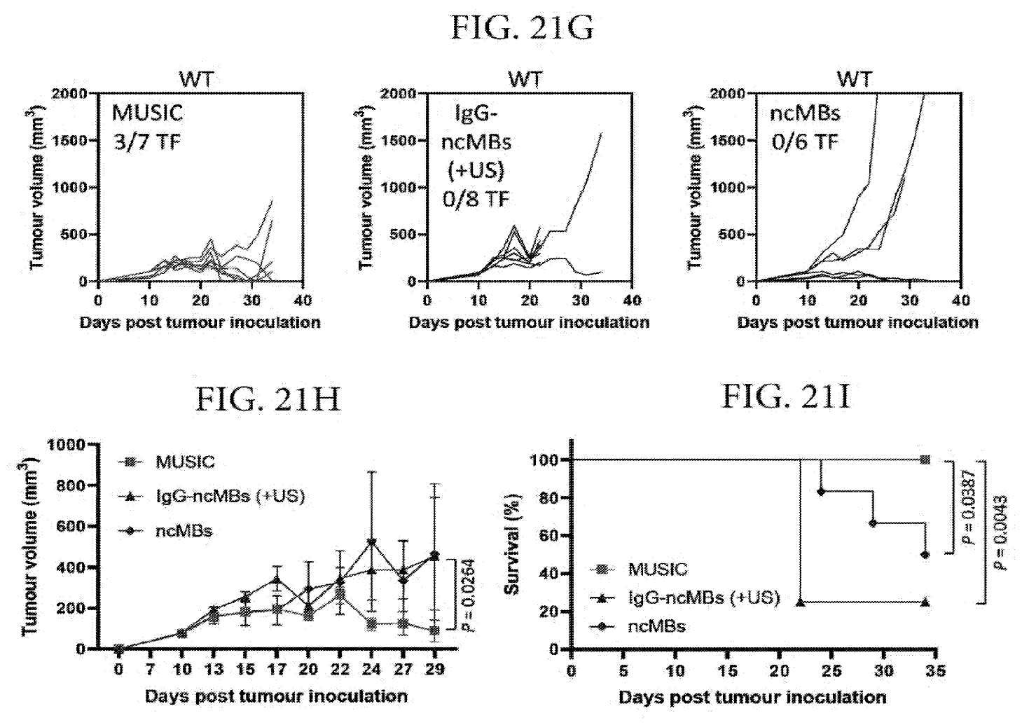

To assess the effect of MUSIC-mediated STING activation on tumor growth, mice were treated with MUSIC, cGAMP, cMBs (+US), or non-targeted IgG-ncMBs (+US) and monitored over time. Tumor sizes were measured every two days using digital calipers, and tumor volume was calculated according to an ellipsoid formula as 0.5×length×width². Mice were sacrificed if any tumor ulceration was observed or if tumors reached 2000 mm³ in volume. The final tumor volume comparison between the MUSIC and control groups was done when the first mice from a control group was sacrificed. Tumor volumes and survival curves are shown in FIGS. 21A-21D. MUSIC given every other day for three treatments led to the most significant tumor growth inhibition and survival benefit. Statistical analysis was done using an unpaired t-test for tumor sizes and Log-rank test for survival curves. A statistical difference was observed in both final tumor volumes and survival between MUSIC treated mice and control mice. Furthermore, 6 out of 10 MUSIC treated mice had complete tumor remission whereas only 2 out of 10 cGAMP treated mice showed a complete response. As expected, no antitumor effect were observed in STING−/− tumor-bearing mice treated with MUSIC, as seen in FIGS. 21E-21F. Furthermore, the effects of ultrasound (US) and CD11b targeting was confirmed by comparing MUSIC with IgG-ncMBs (+US) and ncMBs groups, which showed lower antitumor effects, as seen in FIGS. 21G-21I.

### Example 11. Assessment of Antitumor Immune Memory

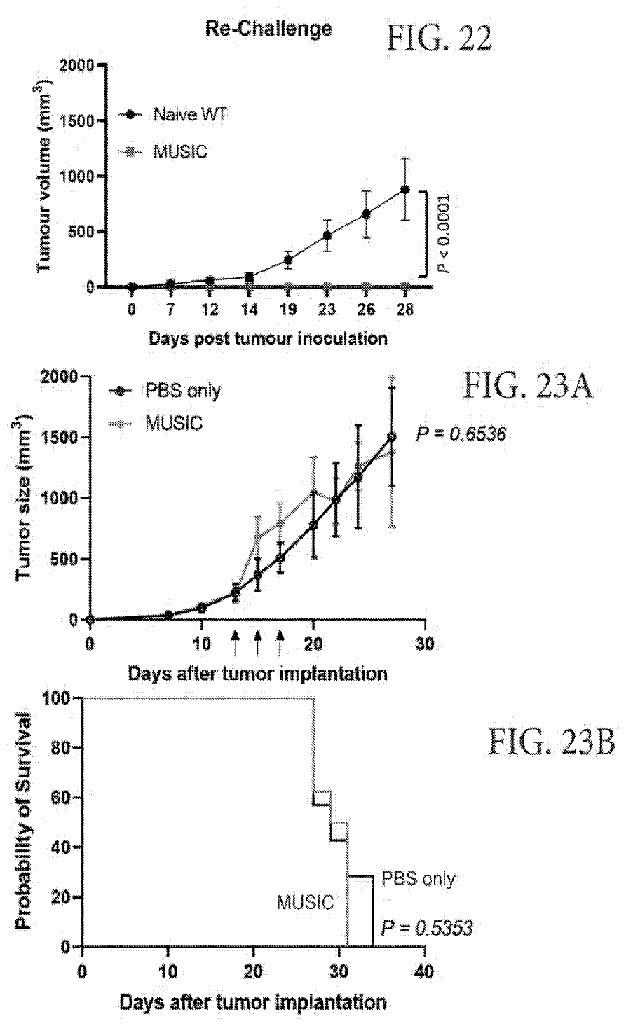

Tumor-free mice from Example 10 were reimplanted with the same EO771 breast cancer cells on the contralateral fat pad of the original tumor for a tumor rechallenging experiment. Non-treated naive mice were also implanted with tumors as a control. MUSIC treated mice showed no tumor growth 19 days after tumor implantation, suggesting that these mice have immune memory against EO771 cells, whereas the naive mice showed obvious tumor growth (FIG. 22). To assess the role of host cell STING activation in mediating MUSIC antitumor responses, STING^{-/-} mice were treated as described above. No statistically significant differences were observed between the MUSIC group and control group for either tumor size (FIG. 23A) or mouse survival (FIG. 23B), suggesting that the observed antitumor efficacy is dependent upon the host cells' STING pathway.

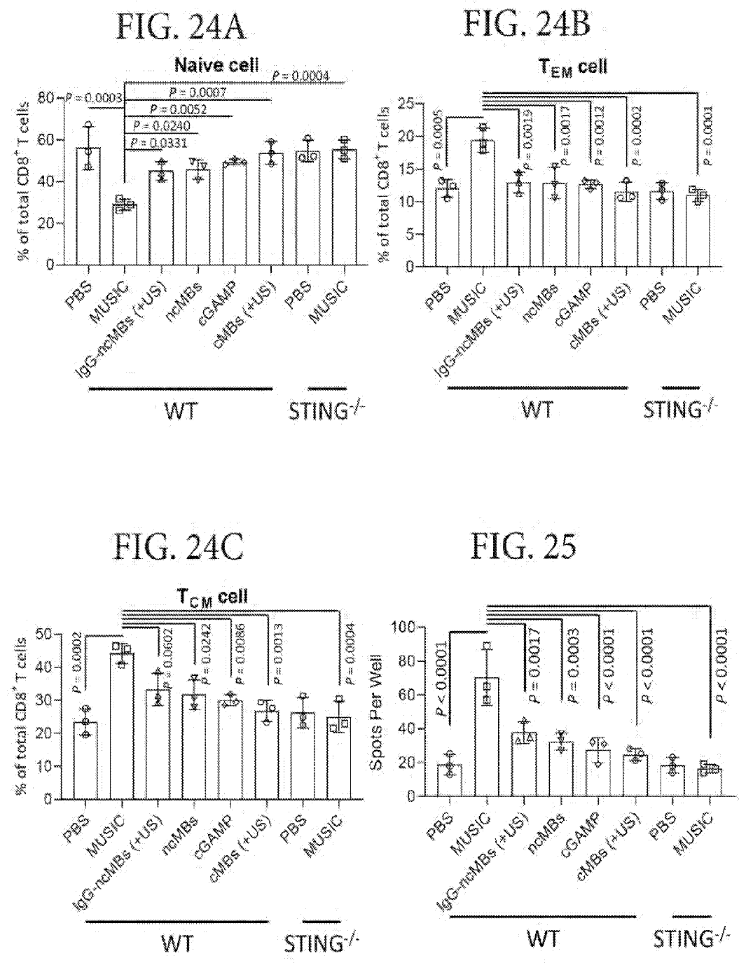

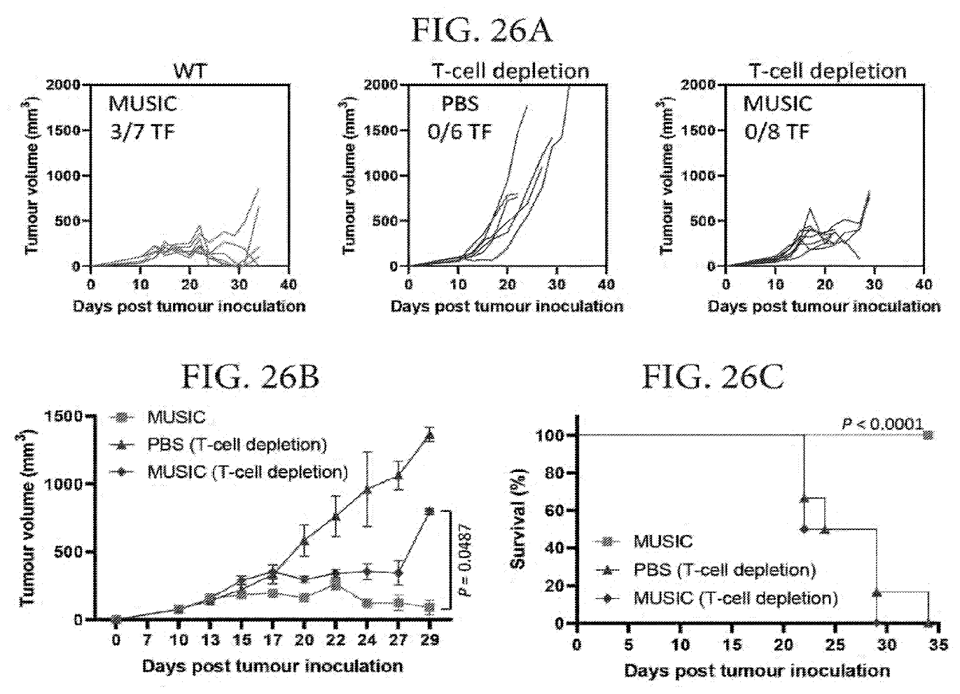

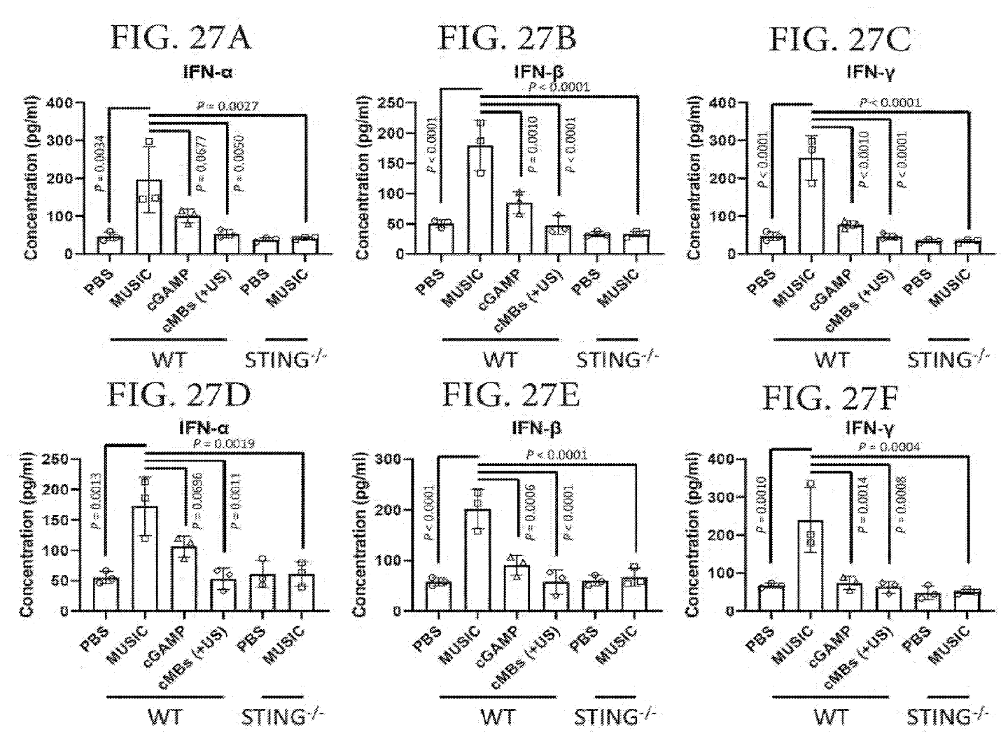

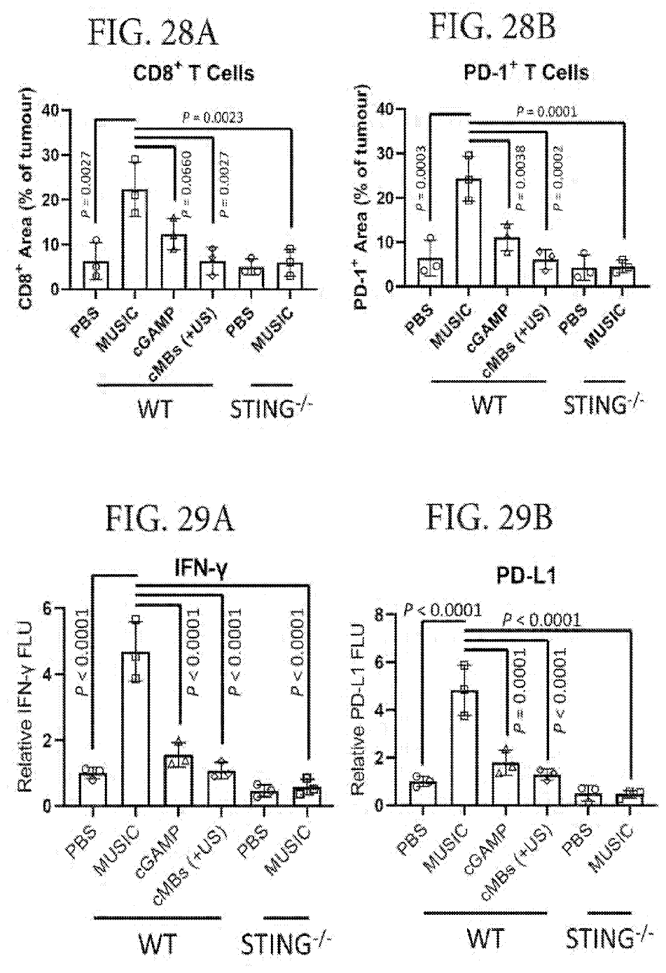

To further characterize MUSIC-mediated antitumor memory, treated tumor tissue samples were analyzed by flow cytometry, as shown in FIGS. 24A-24C, in which a moderate increase in the populations of CD44highCD62Llow effector memory and CD44highCD62Lhigh central memory cells was observed upon MUSIC treatment. In addition, T cells collected from spleens of MUSIC-treated tumor-bearing mice and rechallenged with the EO771 tumor cells in vitro, demonstrated a robust IFN-γ response, as seen in FIG. 25, thus confirming that the local MUSIC treatment generated systemic immune memory in vivo. To further establish the T cell's role in mediating antitumor response of MUSIC, CD8+ T cells from tumor-bearing mice were depleted using an anti-CD8 antibody injected 24 h prior to MUSIC treatment and every 72 h until the end of the experiment. The elimination of CD8+ T cells in these animals abrogated the antitumor effect of MUSIC, resulting in half the mice dying at day 21 (FIGS. 26A-26C). Given that activation of STING in APCs leads to type I IFN production, and CD8+ T cells secrete IFN-γ to produce antitumor effects, type I IFN and IFN-γ levels were measured in tumors (FIGS. 27A-27C) and serum (FIGS. 27D-27F), and were found to be elevated in both upon MUSIC treatment. Since IFN-γ is known to induce the expression of immune checkpoints such as PD-L1, the expression of PD-1 and PD-L1 was measured in T cells and tumor cells, respectively, by comparing the fluorescence intensity from three randomly selected immunostained images (FIGS. 28A-28B).

Over 95% of intratumoral CD8+ T cells in the MUSIC-treated group exhibited elevated expression of PD-1, a marker of cytotoxic T-cell maturation and exhaustion. Tumor tissue PD-L1 expression also correlated positively with IFN-γ level (FIG. 29A-29B). Together, these results demonstrate that MUSIC treatment activates both innate and adaptive immune responses via STING-mediated T cell priming by APCs and provide a rationale for the use of MUSIC in combination with immune checkpoint blockade to generate improved antitumor responses.

### Example 12. Evaluation of Systemic Antitumor Immune Responses Against Metastatic Breast Cancer

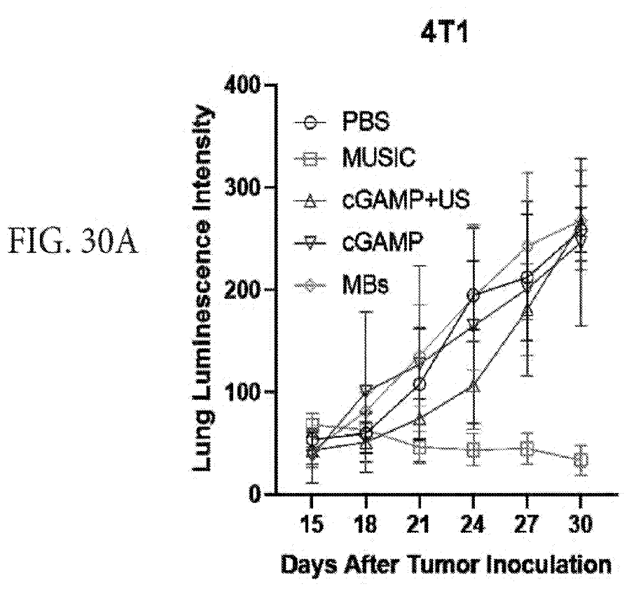

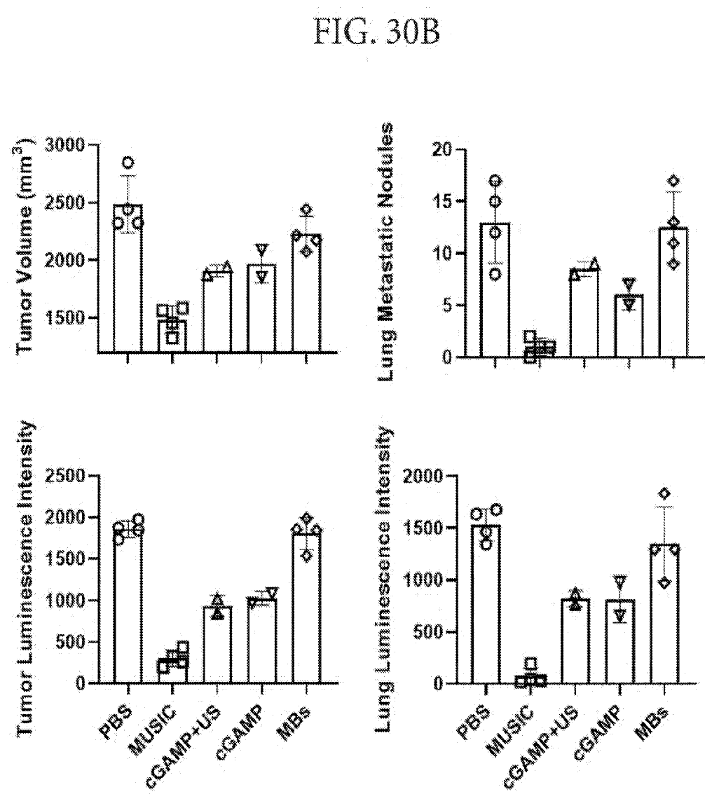

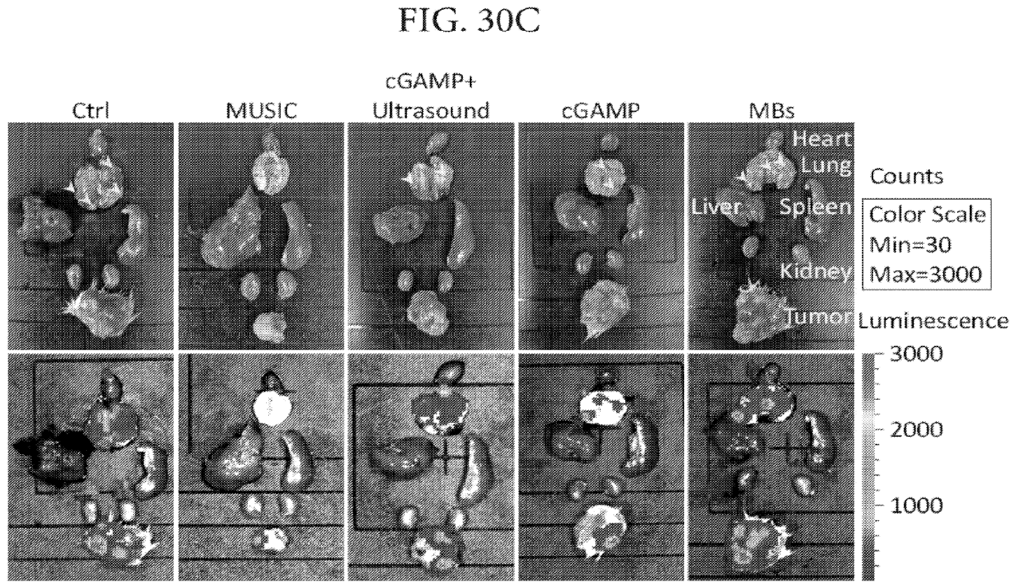

To test whether MUSIC treatment can generate antitumor responses against metastatic breast cancer, metastatic 4T1 tumors were established through orthotopic implantations in the mammary fat pad of Balb/cJ mice using luciferase-expressing 4T1 cells. At 12 days post tumor implantation, mice were intraperitoneally injected with 1.5 mg D-luciferin (SYD LABS™) per 10 g body weight. At 10 min after injection, the presence and metastases of the tumors were monitored by bioluminescence imaging (BLI) using an In Vivo Imaging System (PerkinElmer IVIS® Lumina III). Primary tumors were treated with PBS, cGAMP only, cGAMP+US, cMBs+US, or MUSIC (cMBs+cGAMP+US). The same cGAMP and MB dose was given intratumorally as described in Example 10 and the same US parameters were used. Treatments were given every other day for a total of 3 doses on days 12, 14, and 16. Primary tumor growth and systemic metastatic burden were monitored using digital calipers and bioluminescence imaging respectively. Animals were monitored for 30 days after tumor inoculation, at which point they were sacrificed and their organs collected. Bioluminescence showed that locally treating primary lesion of metastatic 4T1 breast cancer with MUSIC significantly decreased the systemic disease burden including metastases in the lungs when compared to cGAMP alone (FIGS. 30A-30C).

In both syngeneic breast cancer models, MUSIC treatment showed dramatic inhibition of tumor growth, increased mice survival, and a greater percentage of tumor-free mice compared to cGAMP alone. MUSIC treatment also resulted in an 11-fold decrease in metastatic burden when compared to cGAMP alone. These results suggest that MUSIC treatment effectively activates STING in vivo, creating a systemic anti-tumor immune response.

### Example 13. Sensitizing Tumors to PD-1 Blockade with MUSIC

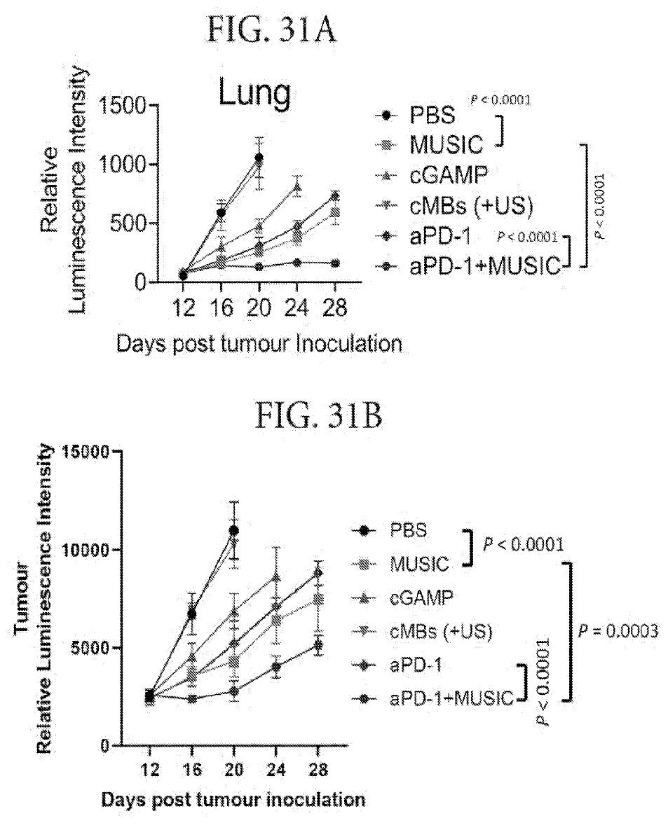

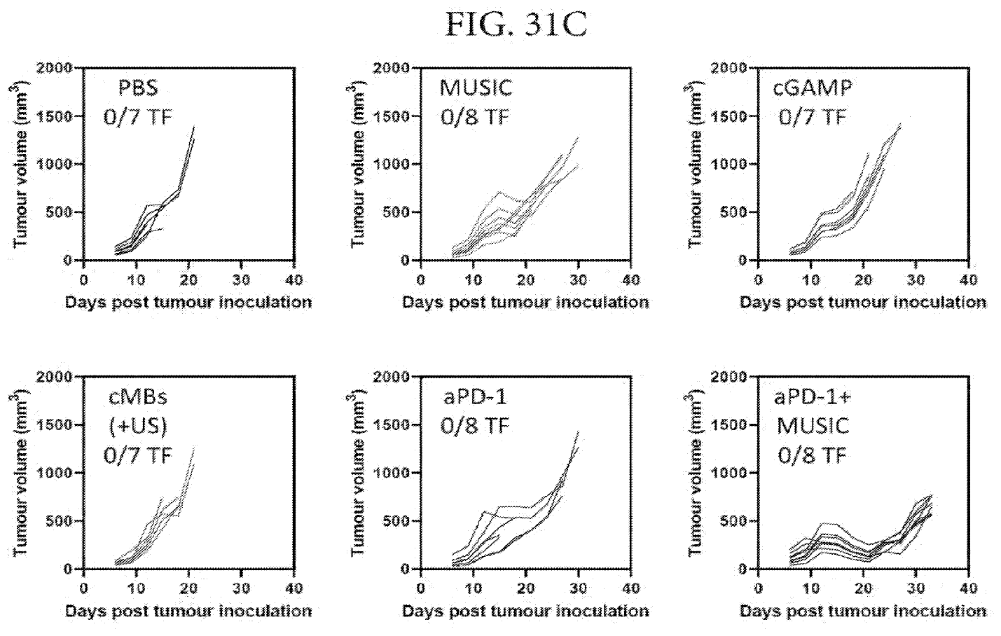

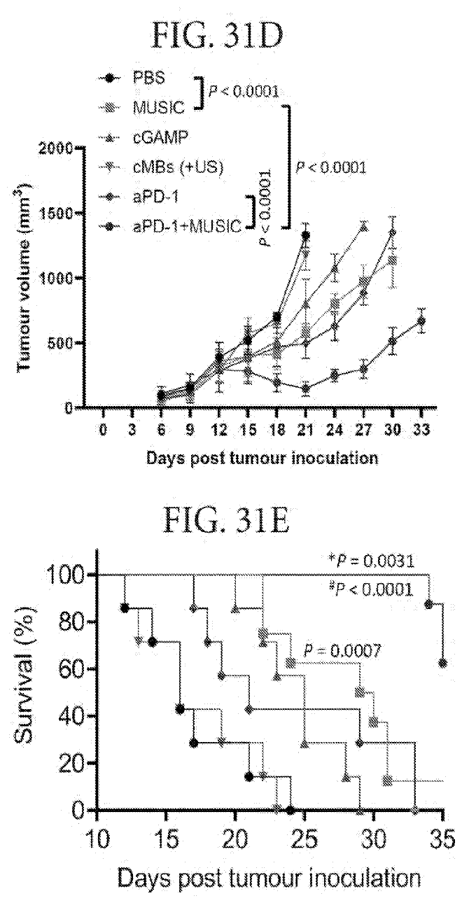

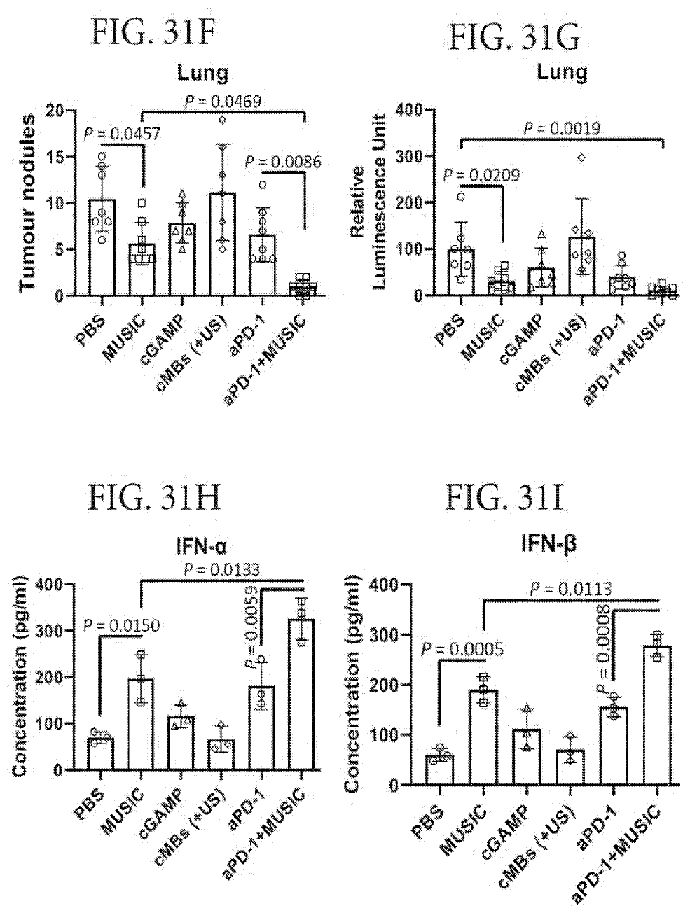

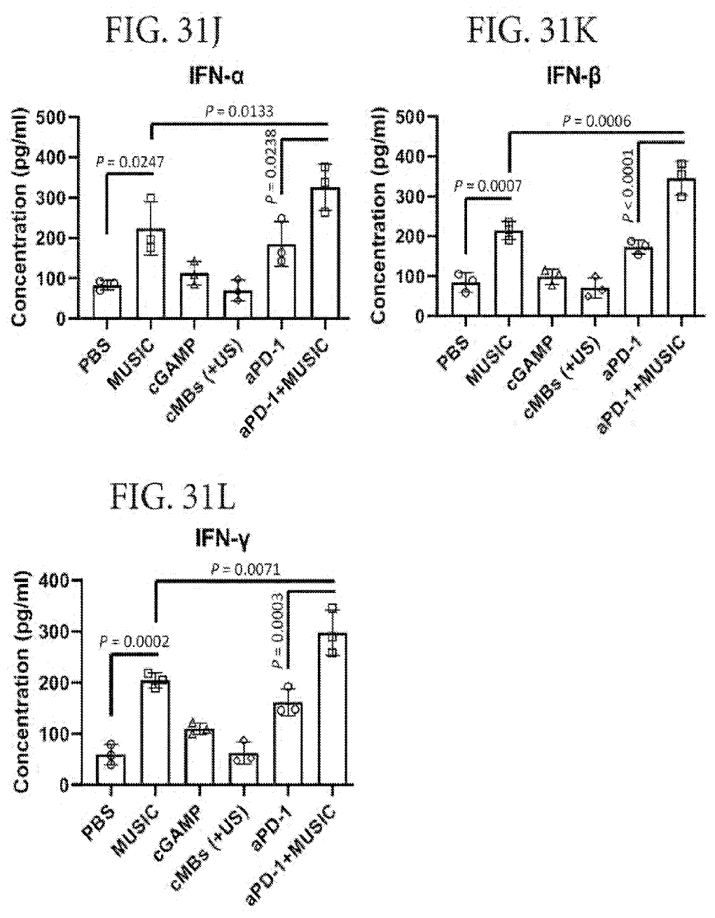

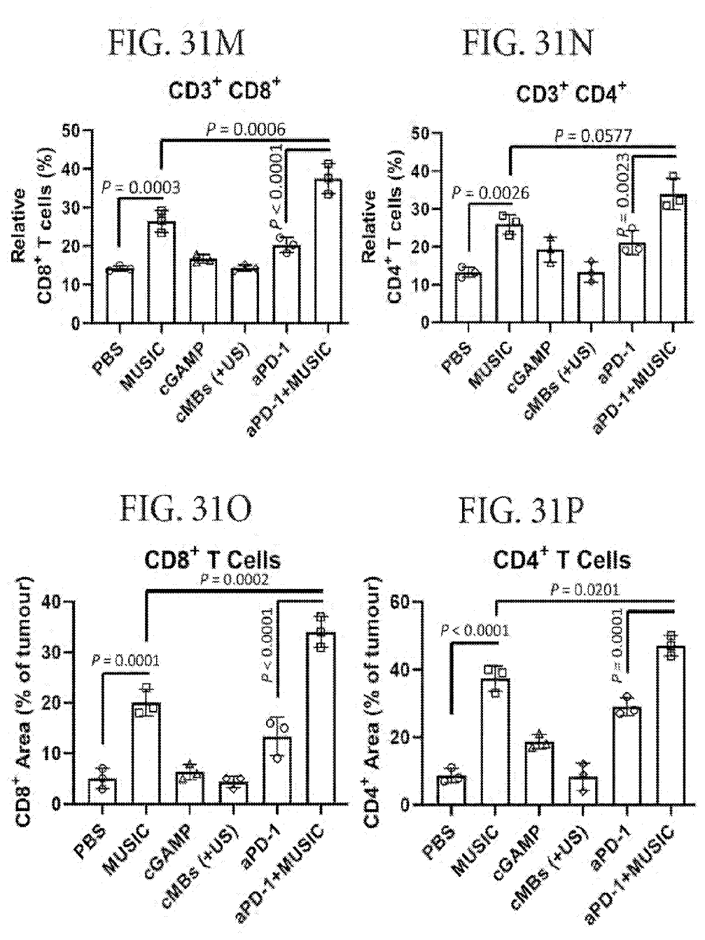

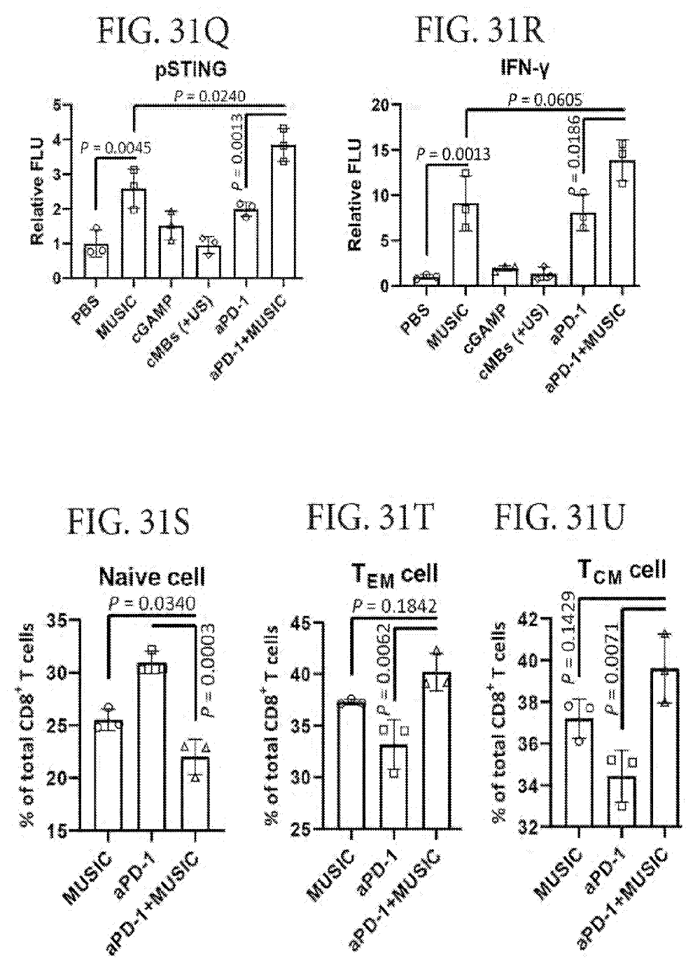

To test whether MUSIC could further sensitize poorly immunogenic tumors to PD-1 blockade, particularly those that are highly aggressive and widely metastatic, spontaneously metastatic murine triple negative 4T1 breast tumor-bearing mice were treated with MUSIC, as in Example 12, in combination with an anti-PD-1 antibody (aPD-1). aPD-1 was administered at a dose of 200 μg/mouse on days 12, 14, 16, 18, 20, and 22. Local MUSIC treatment in combination with systemic aPD-1 administration not only exhibited enhanced primary tumor control, but also significantly decreased systemic disease progression, as compared to either therapy alone, as seen in FIG. 31A-31D. This improved antitumor response in the combination treatment arm directly translated into a superior survival benefit with a 76% increase in median survival as compared to free cGAMP or aPD-1 alone (FIG. 31E). Macroscopic organ imaging and examination revealed significantly reduced lung disease burden and approximately 60% decrease in pulmonary metastatic nodules (FIGS. 31F-31G). To assess the effect of the combination treatment on local and systemic immunity, type I IFN levels were measured in both tumor (FIGS. 31H-31I) and serum (FIGS. 31J-31L) and found to be higher in the combination treatment group compared to the control groups. Both CD4+ and CD8+ T cell infiltrations also increased in the combination treatment group, as seen in FIGS. 31M-31P. Furthermore, the combination treatment showed enhanced phosphorylation of STING (FIG. 31Q) and production of IFN-γ (FIG. 31R) with a low level of Ki67 expression (data not shown). To investigate the effect of the combination treatment on memory T cell responses, tumor infiltrating lymphocytes were collected from different treatment groups. The combination treatment was found to have increased the proportion of CD44^{high}CD62^{low} effector memory and CD44^{high}CD62L^{high} central memory cells (FIGS. 31S-31U). Together, these results demonstrate that MUSIC sensitized poorly immunogenic tumors to PD-1 blockade, and their combination enhanced local and systemic immune activations to produce improved antitumor responses.

### Example 14. Conjugating NBs with Cationic Polymers Allowed Similar cGAMP Loading Efficiency as MBs

In order to bypass the need for intratumoral injection when using 1-3 μm MBs, nanobubbles (NBs) may be used to allow systemic injection and ultrasound-guided therapy. To isolate smaller bubbles and remove any non gas-filled nanoparticles (e.g., liposomes), phospholipids were emulsified with PFB, as described with MBs, but the centrifugation steps post formulation were modified. Specifically, the suspension of emulsified phospholipids was centrifuged at 50 g for 5 min, and the top layer containing large MBs was discarded while the infranatant containing liposomes and NBs was kept and characterized by nanoparticle tracking analysis (NTA) (PARTICLE METRIX™, ZETAVIEW®). SpeDex was conjugated onto NBs and the mixture was then rotated for 2 h, after which thiolated anti-CD11b antibodies were added, as described elsewhere herein. The resulting suspension was centrifuged at 700 g for 5 min and the top layer containing SpeDex-aCD11b NBs was kept while the infranatant was recycled to increase NB yield. The infranatant was re-amalgamated into NBs as described above followed by a final centrifugation at 700 g for 5 min to remove liposomes, antibodies, free lipids and polymers. Both NB suspensions were characterized by NTA and combined.

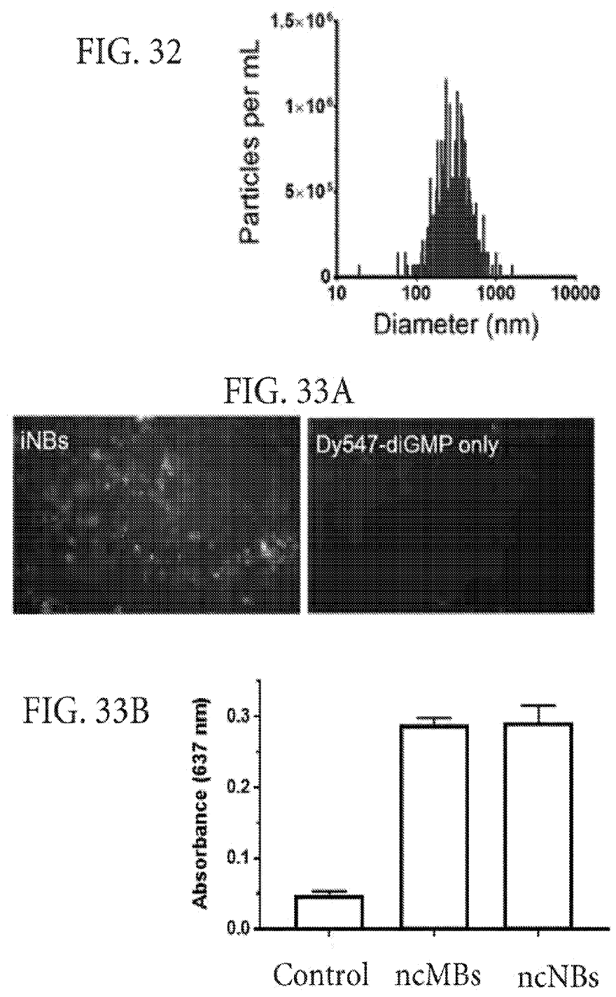

This modified procedure yielded NBs having a mean size of 290 nm and a count of 2.6×10¹¹ NBs/mL. The size distribution is shown in FIG. 32. Conjugation of SpeDex and aCD11b onto NBs was confirmed using flow cytometry by gating the forward scatter (FSC) and side scatter (SSC) signals for the signature shape of bubbles (approximately 84.5% of particles) and by using fluorescent FITC-SpeDex and AF647-aCD11b (data not shown). Flow cytometry showed 100% of NBs conjugated with SpeDex and 91% of NBs conjugated with aCD11b. ncNBs were obtained by loading cGAMP as described elsewhere herein. Both FITC-cGAMP and 2′-O-(6-[DY547]-aminohexylcarbamoyl)-cyclic diguanosine monophosphate (DY547-diGMP), a fluorescent analog of cGAMP, were used to confirm stability and loading efficiency of the dinucleotide by flow cytometry and fluorescence spectrophotometry (data not shown). cGAMP loading was highly efficient with 7.1×10⁶ cGAMP molecules loaded per NB, corresponding to a 78% loading efficiency or 0.027 μg of cGAMP/μm². The loading efficiency of cGAMP in ncNBs was very close to the loading value obtained with ncMBs (0.033 μg/m²).

### Example 15. Sonoporation of Macrophages with Targeted ncNBs Allows the Cytosolic Delivery of cGAMP

To confirm cytosolic delivery in APCs, ncNBs were incubated with human THP-1 macrophages at a ratio of 500 ncNBs/cell for 10 min, followed by a washing step to remove any unbound ncNBs. Sonoporation was performed using a plane wave single element transducer transmitting at 1 MHz (SONITRON® GTS, 15 mm diameter) at a power of 1 W/cm² with 20% duty cycle for 60 seconds. Successful cytosolic delivery was confirmed by fluorescence microscopy when compared to incubation with Dy547-diGMP alone, as shown in FIG. 33A). STING activation was confirmed using THP1-BLUE NF-κB cells (INVIVOGEN®) that have been modified with a NF-κB-inducible secreted embryonic alkaline phosphatase (SEAP) reporter that is measured with a QUANTI-BLUE™ assay following sonoporation. No difference was observed between STING activation by ncNBs or ncMBs relative to a control of non-treated cells, as shown in FIG. 33B.

### Example 16. Systemically Administered ncNBs Accumulate in the Tumor and can be Imaged by US

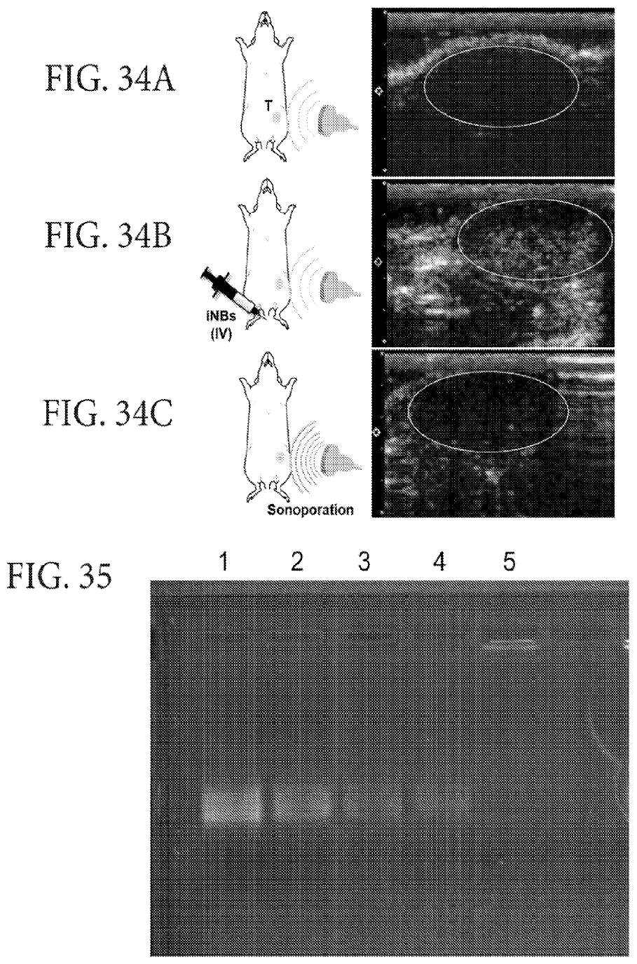

To confirm that the ncNBs are an acoustically responsive material that can reach the tumor micro-environment and activate STING in APCs under US guidance, a clinical US scanner was used to image ncNBs following IV injection into the orthotopic syngeneic murine breast cancer model produced from implantation of EO771 breast cancer cells, described elsewhere herein. At 7 days after inoculation, mice were anesthetized with isoflurane and a catheter tubing was placed in the lateral tail vein for injection of ncMBs. A 50 μL suspension containing 5×10⁹ ncNBs was injected followed by a 50 μL flush of saline. Mice breast tumors were imaged before, during and after ncNBs IV injection using a clinical US scanner (SIEMENS® ACUSON® Sequoia with a 18H6 transducer), as shown in FIGS. 34A-34C. Signal in the tumor increased dramatically as early as 90 s after injection, as seen in FIG. 34B, confirming accumulation of ncNBs in the tumor). Upon increase of the mechanical index from 0.04 to 0.68, all signal in the tumor disappeared, as seen in FIG. 34C, which demonstrates that a clinical scanner could be used to sonoporate APCs targeted with ncNBs.

### Example 17. Loading of SpeDex MBs with mRNA

To confirm that SpeDex microbubbles (MBs) can be loaded with nucleic acids in addition to CDNs, 9 million MBs were added to a solution of mRNA in PBS and mixed for 15 min. Afterwards, the solution was diluted with TRITRACK™ 6× loading buffer (90% glycerol) to obtain a 15% glycerol concentration. The entire volume was loaded onto a 1% agarose gel and ran at 80 V for 40 minutes. The gel is shown in FIG. 35 (Lane 1=200 ng mRNA, Lane 2=100 ng mRNA, Lane 3=50 ng mRNA, Lane 4=25 ng mRNA, Lane 5=200 ng mRNA complexed to SpeDex MBs). As evident by the missing band, the 9 million SpeDex MBs were able to completely bind 200 ng mRNA. This equates to a loading of at least 1.57×10³ pg/μm² of mRNA of SpeDex MB.

All data presented in the disclosed examples are shown as mean±standard deviation (s.d.) or mean±standard error of mean (s.e.m.) from at least triplicate conditions unless otherwise indicated. Each experiment was repeated independently at least three times unless otherwise indicated. Statistical analyses included unpaired Student t-test and one-way ANOVA with Tukey's or Dunnett's multiple comparisons test, as appropriate. Survival was determined for mice in every group by the Kaplan-Meier method and compared by the log-rank (Mantel-Cox) test. The P values of less than 0.05 were considered to indicate statistical significance. No animals were excluded from the analyses.

All documents cited herein, including patents, patent applications, and publications, are herein incorporated by reference in their entirety just as if each specific mention of the document had explicitly stated the document to be incorporated by reference in its entirety. The relevance of the material incorporated by reference to the present disclosure is to be understood from context, including the specific context in which the incorporated document was mentioned.

It is understood that the disclosure herein contemplates any possible combination of the various aspects described herein even if not explicitly exemplified, unless indicated otherwise, explicitly or by context (e.g., where various aspects would be understood to be physically incompatible). The disclosure also contemplates combinations of the various aspects described herein with relevant features that are well known, routine, or conventional to those of ordinary skill in the art.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.” The term “including” does not necessarily imply that additional elements beyond those recited must be present.

As used herein, “about” or “approximately” will be understood by persons of ordinary skill and will vary to some extent depending on the context in which it is used. When referring to a number or a numerical range, the terms generally mean that the number or numerical range referred to is an approximation within experimental variability (or within statistical experimental error), and, thus, the number or numerical range may vary from, for example, between 1% and 20% of the stated number or numerical range. In some aspects, “about” indicates a value within 20% of the stated value. In more preferred aspects, “about” indicates a value within 10% of the stated value. In even more preferred aspects, “about” indicates a value within 1% of the stated value. The variation encompassed by about may be above or below the recited number or range, unless indicated otherwise, explicitly or by context.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in aspects of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

It should be noted that whenever a value or range of values of a parameter are recited, it is intended that values and ranges intermediate to the recited values are also intended to be part of this disclosure.

As used herein, the term “at least” or “no less than” prior to a value or series of values is understood to include the value adjacent to the term “at least” or “no less than” and all subsequent logical values or integers that could logically be included, as understood from context. When “at least” or “no less than” is present before a series of values or a range of values, it is understood that “at least” or “no less than” can modify each of the values in the series or range as just described. The term “down to” will be understood to mean the same as “at least” or “no less than” and will also include the value adjacent to the term “down to” unless indicated otherwise, explicitly or by context.

As used herein, “no more than” or “no greater than” are understood to include the value adjacent to the phrase (unless indicated otherwise explicitly or by context) and all lower values or integers that could logically be included, as understood from context (down to and including zero if negative values are not possible, down to but not including zero if the values must be positive, or down to and including 1 if the value must be a positive integer). When “no more than” or “no greater” is present before a series of values or a range of values, it is understood that “no more than” or “no greater than” can modify each of the values in the series or range as just described. The term “up to” will be understood to mean the same as “no more than” or “no greater than” and will also include the value adjacent to the term “up to” unless indicated otherwise, explicitly or by context.

Where a range of values is provided, it is understood that all intervening values are encompassed by the disclosure as well as the upper and lower limits of the range. For example, recitation of a range would be inferred to disclose each intervening value (e.g., to the tenth of the unit of the lower limit unless the context clearly dictates otherwise) between the upper and lower limit of that range and any other stated or intervening value in that stated range. Ranges excluding either or both of the upper and lower limits are also contemplated by the disclosure of a range.

What is claimed is:

1. A method of targeted in vitro or in vivo drug delivery using sonoporation, the method comprising (i) administering to one or more target cells a composition comprising microbubbles loaded with a payload and (ii) administering an ultrasound stimulus to the one or more target cells, wherein the ultrasound stimulus is effective to sonoporate the one or more target cells.

2. The method of claim 1, wherein the payload comprises an agonist for activating the Stimulator of Interferon Genes (STING) signaling pathway within the one or more target cells, optionally wherein the agonist is a cyclic dinucleotide.

3. The method of claim 1, wherein the payload comprises a cyclic dinucleotide for inducing or enhancing Type 1 Interferon production within one or more cells.

4. The method of any one of claims 1-3, wherein the method is an in vivo method comprising administering the microbubble composition and the ultrasound stimulus to a subject.

5. The method of any one of the preceding claims, wherein the one or more target cells comprise cancer cells.

6. The method of any one of the preceding claims, wherein the one or more target cells comprise immune cells.

7. The method of claim 6, wherein the immune cells comprise professional antigen-presenting cells (APCs).

8. The method of claim 7, wherein the APCs comprise macrophages

9. The method of claim 7 or 8, wherein the APCs comprise dendritic cells.

10. The method of any one of the preceding claims, wherein the microbubbles comprise targeting molecules on the external surfaces of the microbubbles, the targeting molecules being effective to bind the one or more target cells.

11. The method of claim 10, wherein the targeting molecules comprise antibodies.

12. The method of claim 10 or 11, wherein the targeting molecules bind CD11b.

13. The method of any one of the preceding claims, wherein the ultrasound stimulus is administered at about 1-2 W/cm², optionally with 50% duty cycle.

14. The method of any one of the preceding claims, wherein the ultrasound stimulus is administered for between about at least about 30-60 seconds.

15. The method of any one of the preceding claims, wherein the one or more target cells are exposed to the microbubbles for at least about 10 minutes prior to administering the ultrasound stimulus.

16. The method of any one of the preceding claims, further comprising using ultrasound to visualize the microbubbles prior to applying the ultrasound stimulus effective to sonoporate the cell membrane, wherein the intensity of the ultrasound used to visualize the microbubbles is less than the intensity of the ultrasound stimulus.

17. The method of any one of the preceding claims, wherein the microbubbles are decorated with spermine, the payload being non-covalently bound to the spermine.

18. The method of any one of the preceding claims, wherein the microbubbles are decorated with spermine-dextran conjugates, the payload being non-covalently bound to the spermine within the spermine-dextran conjugates.

19. The method of any one of the preceding claims, wherein the microbubbles comprise gas cores comprising a perfluorocarbon, optionally wherein the perfluorocarbon is decafluorobutane.

20. The method of any one of the preceding claims, wherein the microbubbles comprise shells comprising phospholipids, optionally wherein the phospholipids comprise one or both of 1,2-Distearoyl-sn-Glycero-3-Phosphocholine (DSPC) and 1,2-Distearoyl-sn-Glycero-3-Phosphoethanolamine (DSPE) lipids.

21. The method of any one of the preceding claims, wherein the microbubbles comprise surfactant shells comprising PEGylated molecules.

22. The method of any one of the preceding claims, wherein the average microbubble size of the microbubble composition is between about 1 μm and about 10 μm.

23. The method of claim 22, wherein the average microbubble size of the microbubble composition is between about 1 μm and about 5 μm

24. The method of claim 23, wherein the average microbubble size of the microbubble composition is about 3 μm.

25. The method of any one of claims 1-22, wherein the microbubbles are primarily nanobubbles.

26. The method of claim 25, wherein the microbubbles are entirely nanobubbles.

27. The method of claim 25 or 26, wherein the average microbubble size of the microbubble composition is between about 100 nm and 700 nm.

28. The method of claim 27, wherein the average microbubble size of the microbubble composition is between about 200 nm and 600 nm.

29. The method of claim 28, wherein the average microbubble size of the microbubble composition is between about 300 nm and 500 nm.

30. The method of any one of the preceding claims, wherein the microbubble composition comprises microbubbles with biodegradable linkers operably positioned between an exterior surface of a shell of the microbubble and the payload, optionally wherein the biodegradable linker is joining a spermine to a dextran or a spermine to another spermine.

31. The method of any one of the preceding claims, wherein the payload comprises cyclic guanosine monophosphate-adenosine monophosphate (cGAMP).

32. The method of any one of claims 1, 2, or 4-31, wherein the method is an in vitro method.

33. The method of claim 32, wherein the composition comprising microbubbles is incubated with the one or more target cells at a concentration of at least about 5, 10, 15, 20, 25, or 30 microbubbles/cell.

34. The method of claim 32 or 33, wherein the step of administering to the one or more target cells a composition comprising microbubbles comprising mixing the composition with the one or more target cells in solution.

35. The method of claim 32 or 33, wherein the one or more target cells are adhered to a surface and wherein the step of administering to the one or more target cells a composition comprising microbubbles comprises exposing the surface to the composition comprising microbubbles such that the one or more cells are positioned over the microbubbles.

36. A method of treating cancer in a subject in need thereof, the method comprising performing the targeted drug delivery method of any one of claims 4-31, wherein administering the microbubble composition to the one or more target cells comprises administering the microbubble composition and the ultrasound stimulus to the subject, and wherein the payload comprises a cyclic dinucleotide.

37. The method of claim 36, wherein the subject has been diagnosed with cancer.

38. The method of claim 36 or 37, wherein the subject has a tumor.

39. The method of claim 38, wherein the subject has one or more metastases.

40. The method of claim 38, wherein the microbubble composition is administered intratumorally.

41. The method of any one of claims 36-39, wherein the microbubble composition is administered systemically.

42. The method of claim 41, wherein the microbubble composition is administered intravenously.

43. The method of claim 41 or 42, wherein the microbubble composition is a nanobubble composition.

44. The method of any one of claims 36-43, wherein administering the microbubble composition to the subject comprises administering multiple doses of the microbubble composition to the subject and administering the ultrasound stimulus to the subject comprises administering ultrasound stimulus effective to sonoporate the one or more target cells after each dose.

45. The method of claim 44, wherein the multiple doses are administered at least one day apart.

46. The method of any one of claims 36-45, wherein the administration results in an increase in expression of IFN-α, IFN-β, and/or IFN-γ within the one or more target cells.

47. The method of any one of claims 36-46, wherein the administration results in an increase in serum levels of IFN-α, IFN-β, and/or IFN-γ.

48. The method of any one of claims 36-47, wherein the administration results in nuclear localization of nuclear translocation of phosphorylated IRF3 (pIRF3) and/or NF-κB p65 in the one or more target cells.

49. The method of any one of claims 38-48, wherein the administration results in increased recruitment of CD8⁺ and CD4⁺ T cells within the tumor.

50. The method of any one of claims 37-49, wherein the administration results in an increased number of effector memory T-cells and/or central memory T-cells that are specific to cancer cells within the subject, optionally wherein an increased number of effector memory T-cells and/or central memory T-cells are found within the tumor.

51. The method of any one of claims 38-50, wherein the administration results in a decrease in tumor size.

52. The method of claim 51, wherein the administration results in the eradication of the tumor.

53. The method of any one of claims 36-52, wherein the administration prevents or reduces the likelihood of future metastases.

54. The method of any one of claims 37-53, wherein the administration prevents or reduces the likelihood of recurrence of the cancer in the subject.

55. The method of any one of claims 36-54, wherein the method further comprises treating the subject with immune checkpoint therapy.

56. The method of claim 55, wherein the immune checkpoint therapy comprises administering to the subject inhibitors that target CTLA4, PD-1, PD-L1, and/or CD47.

57. A microbubble composition for therapeutic drug delivery, the microbubble composition comprising:
a plurality of microbubbles, wherein the microbubbles each comprise a gas core encapsulated by a surfactant shell, and wherein a plurality of cationic polymers are associated with the external surface of the surfactant shell of each microbubble; and
a plurality of cyclic dinucleotides, wherein the cyclic dinucleotides are non-covalently bound to the cationic polymers on the external surface of the microbubbles.

58. The microbubble composition of claim 30, wherein the cationic polymers comprise polyamines.

59. The microbubble composition of claim 31, wherein the polyamines comprise spermines.

60. The microbubble composition of claim 32, wherein the spermines are conjugated to dextrans, optionally wherein multiple spermines are conjugated to each dextran.

61. The microbubble composition of any one of claims 30-33, wherein the plurality of cyclic dinucleotides comprises cyclic guanosine monophosphate-adenosine monophosphate (cGAMP).

62. A method of making the microbubble composition of anyone of claims 57-61, the method comprising:
associating the cationic polymers with the microbubbles; and
loading the cyclic dinucleotides onto the microbubbles after the cationic polymers have been associated.

63. A method of making the microbubble composition of anyone of claims 57-61, the method comprising:
binding the cyclic dinucleotides to the cationic polymers to form nanocomplexes; and
loading the Nanocomplexes onto the microbubbles.

64. The microbubble composition formed by claim 62 or 63.

65. Use of the microbubble composition of any one of claims 57-61 or 64 in any one of the methods of claim 1-56.

66. The method of claim 1, wherein the payload comprises mRNA.

67. The method of claim 1, wherein the payload comprises DNA, optionally plasmid DNA (pDNA).
